(19) United States
(12) Patent Application Publication
(10) Pub. No.: US 2010/0120039 A1
(43) Pub. Date: May 13, 2010

(54) METHODS AND COMPOSITIONS IN BREAST CANCER THERAPY RESISTANCE

(76) Inventor: Suzanne Fuqua, Sugar Land, TX (US)

Correspondence Address: FULBRIGHT & JAWORSKI, LLP, 1301 MCKINNEY, SUITE 5100, HOUSTON, TX, 77010-3095, US

(21) Appl. No.: 12/548,995

(22) Filed: Aug. 27, 2009

Related U.S. Application Data
(60) Provisional application No. 61/092,196, filed on Aug. 27, 2008.

(51) Int. Cl. C12Q 1/68; G01N 33/574; C07K 7/06; C07K 7/08

(52) U.S. Cl. 435/6; 435/7.23; 530/326; 530/330; 530/328; 530/327

(57) ABSTRACT

The present invention is directed to methods and/or compositions regarding a specific mutation in estrogen receptor alpha and their use for identifying resistance to breast cancer therapy and/or treatment therefor. More specifically, the present invention concerns presence or absence of an A908G mutation in an estrogen receptor alpha nucleic acid sequence, and/or the corresponding K303R mutation in the estrogen receptor alpha polypeptide sequence, for example, as a predictive marker for resistance to breast cancer therapy. Therapeutic embodiments for overcoming the resistance are also provided.

This application claims priority to U.S. Provisional Patent Application Ser. No. 61/092,196, filed Aug. 27, 2008, which application is incorporated by reference herein in its entirety.

This invention was developed with funds from the United States Government pursuant to National Institutes of Health/National Cancer Institute Grant No. RO1CA72038. The United States Government has certain rights in the invention.

### FIELD OF THE INVENTION

The present invention is directed to at least the fields of cancer and molecular genetics. Specifically, the present invention is directed to the determination of resistance to breast cancer therapy. More specifically, the present invention is directed to a mutation in estrogen receptor alpha (ER) and its association with resistance to breast cancer therapy. Therapeutic embodiments are also disclosed.

### BACKGROUND OF THE INVENTION

Estrogens play a crucial role in regulating the growth and differentiation of normal breast epithelium and also of breast cancers, with approximately two-thirds of all breast cancers dependent for their growth on a functional estrogen receptor α (ER α). ER α is a member of the nuclear hormone receptor superfamily that regulates transcription of ER target genes by binding with specific estrogen response elements (Mangelsdorf et al., 1995). However, ER α also regulates the expression of many genes without direct binding to DNA. This occurs via protein-protein interactions with other transcriptions factors, such as activator protein-1, and with extranuclear signalling complexes that, in turn, modulate downstream gene expression (O'Malley, 2006; Bjornstrom and Sjoberg, 2005). Therapeutic strategies directed at inhibiting the action of ER α using antiestrogens, such as tamoxifen (Tam), or reducing estrogen levels using aromatase inhibitors (AIs), are the standard therapies offered to women with ER α-positive cancer. However, not all patients who have ER positive tumors respond to endocrine therapies (termed de novo resistance), and a large number of patients who do respond will eventually develop disease progression or recurrence while on therapy (acquired resistance).

In previous work the present inventor and others identified an A to G somatic mutation at ER α nucleotide 908 (A908G) in early premalignant breast lesions (Fuqua et al., 2000). This transition introduces a lysine to arginine substitution at residue 303 (K303R ER α) within exon 4, at the border between the hinge and the hormone-binding domain of the receptor; the mutation confers increased sensitivity to subphysiologic levels of estrogen (Fuqua et al., 2000). The mutation resides at major post-translational modifications sties (acetylation, ubiquitination, methylation, sumoylation (Sentis et al., 2008; Subramanian et al., 2005) adjacent to a protein kinase A (PKA) phosphorylation site at serine residue 305 (S305). Cui et al. have demonstrated that this naturally-occurring mutation is a more efficient substrate for phosphorylation by PKA and is hypoacetylated, which subsequently alters estrogen sensitivity (Cui et al., 2004). Michalides et al. have suggested that phosphorylation of ER α S305 by PKA induces a switch from antagonistic to agonistic effects of Tam, which induces resistance to this antiestrogen (Michalides et al., 2004). It has also been shown that the ER α S305 site can be an in vivo substrate for p21-activated kinase 1 (PAK 1)-mediated phosphorylation, and that activation of ER α S305 might confer a conformational change that allows for a better interaction with ligands such as Tam (Wang et al., 2002; Rayala et al., 2006).

It is known that estrogen regulation of breast cancer cell growth can also be modulated by complex interactions with a variety of peptide growth factors. A large body of evidence supports the idea that rapid membrane effects of ER α may activate various components of growth factor tyrosine kinase signalling, such as that from insulin-like growth factor-IR (IGF-IR), epidermal growth factor receptor (EGFR), and c-erbB2/HER2 (Levin, 2003; Fan et al., 2007; Song and Santen, 2006). Furthermore, the kinase cascade signalling initiated by growth factor receptors can activate ER α (termed ligand-independent effects) and its coregulatory proteins, causing an interdependent loop of cross-talk that leads to enhanced tumor cell survival and proliferation (Lee et al., 2001; Nicholson et al., 1999; Schiff et al., 2003; Schiff et al., 2004). Moreover, several preclinical and clinical studies suggest that overexpression of EGFR or HER2, and/or high levels of phosphorylated Akt and extracellular signal-regulated kinases (ERKs) in breast cancers contribute to Tam resistance (Arpino et al., 2004; Berry et al., 2000; Shou et al., 2004; Perez-Tenorio and Stal, 2002; Gee et al., 2001; Nicholson et al., 1988).

The present invention provides a solution for a long-felt need in the art to identify individuals that are or will become resistant to therapy and/or a solution for treatment for resistant individuals.

### SUMMARY OF THE INVENTION

The present invention concerns methods and/or compositions for identifying individuals that are resistant, will become resistant, are at risk for becoming resistant, or are susceptible to becoming resistant to hormonal breast cancer therapy, in addition to therapies for the individuals. In particular, the present invention concerns methods and/or compositions related to a particular mutation in the estrogen receptor alpha that identifies individuals that are resistant, will become resistant, are at risk for becoming resistant, or are susceptible to becoming resistant to hormonal breast cancer therapy. Although the breast cancer therapy may be of any type, in specific embodiments the breast cancer therapy comprises chemotherapy and/or hormonal therapy, including antiestrogens, aromatase inhibitors, and so forth.

In alternative embodiments, the present invention concerns methods and/or compositions for identifying in individuals a particular mutation in the estrogen receptor alpha that render the individual more sensitive to chemotherapy than an individual that lacks the particular mutation. In a specific case, the mutant cells are more sensitive to chemotherapy because they are replicating at a faster rate.

In specific embodiments, a particular mutation in the estrogen receptor alpha identifies an individual that is resistant, will become resistant, or is at risk for becoming resistant to one or more hormonal breast cancer therapies and that is sensitive to one or more particular chemotherapies.

In certain embodiments of the invention, the present invention concerns a particular mutation in estrogen receptor alpha ERα A908G in the nucleic acid sequence, which corresponds to K303R in the amino acid sequence. In particular, the inventor has previously identified a somatic mutation at nucleotide 908 of ERα (A908G) in premalignant and invasive breast cancers. This mutation results in a lysine to arginine transition at residue 303 (K303R) that confers hypersensitivity to estrogen (Fuqua et al., 2000; Herynk et al., 2007). The present invention concerns the association of this mutation also with resistance to hormonal breast cancer therapy.

About 75% of breast cancer patients have ERα-positive tumors, but not all patients who have ER+ tumors respond to endocrine therapies, for example. The K303R ERα mutant is hypersensitive to estrogen, and a more efficient substrate for PKA and AKT phosphorylation at S305 (Cui et al., 2004). Phosphorylation at ERα S305 can modify receptor sensitivity to anti-hormonal therapies (Michalides et al., 2004; Zwart et al., 2007). Estrogen regulation of breast cancer cell growth can be modulated by complex interaction with a variety of peptide growth factor receptors (Lee et al., 2001; Nicholson et al., 1999). Overexpression of HER2 and high levels of phosphorylated Akt or ERK1/2 in breast cancers contributes to some forms of tamoxifen resistance (Arpino et al., 2004; Perez-Tenorio et al., 2002; Gee et al., 2001).

In certain embodiments of the invention, the K303R mutation adapts ER α for enhanced reception of intracellular signal transduction, which leads to antiestrogen resistance. As described herein, an experimental model of MCF-7 breast cancer cells was stably transfected with either wild-type (WT) or the K303R mutant ER α Cells expressing the estrogen hypersensitive K303R ER α mutant showed elevated levels of growth factor signalling and enhanced cross-talk between the mutant and the HER2 growth factor receptor. These results indicate that the presence of the A908G ER α somatic mutation is useful as a predictive marker of hormonal response in patients whose tumors exploit ER α and/or growth factor cross-talk to evade treatment.

In another embodiment of the invention, the therapy to which the individual is resistant is aromatase inhibitor therapy. Emerging data indicates the superiority of AIs over tamoxifen. However, many patients still exhibit de novo and acquired resistance to AIs (AI^{R}). To study AI^{R}, a number of investigators have generated estrogen-deprived MCF-7 models (Martin et al., 2005; Nicholson et al., 2004; Sabnis et al., 2005; Santen et al., 2005) or breast cancer cells resistant to aromatase inhibitors generated with long-term treatment (Jelovac et al., 2005; Chen et al., 2006). In one embodiment of the invention, resistance in vitro results from estrogen hypersensitivity or estrogen-independent activation of ERα. PI3K/Akt pathway plays a major role in breast cancer, with up-regulation associated with a more aggressive clinical phenotype and a worse clinical outcome for endocrine-treated patients (Song et al., 2005; Kim et al., 2005).

In an embodiment of the present invention there is an isolated estrogen receptor alpha nucleic acid sequence comprising an A908G mutation. In another embodiment of the present invention there is an isolated estrogen receptor alpha amino acid sequence comprising a K303R substitution. In an additional embodiment, there is a peptide that comprises the region of ERα that comprises the mutation site. The region may be of any size so long as it is capable of allowing the individual to which it is administered to overcome the breast cancer therapy resistance, and methods of testing peptides are described herein. The peptide may be at least 10 amino acids in length and no more than 25 amino acids in length, in certain cases The peptide may be identical to the corresponding region of ERα, or it may have one or more alterations. In one embodiment, there is an alteration at the mutated site, corresponding to amino acid 303. In particular cases, the peptide may be able to be phosphorylated, for example at the corresponding 305 amino acid, whereas in other cases it may not be able to be phosphorylated at the corresponding 305 amino acid. In some cases, one or more of the amino acids of the peptide is modified compared to the corresponding wild type region, for example K302/K303 acetylated, methylated, sumoylated, and so forth.

In an additional embodiment of the present invention there is a method of detecting susceptibility to development of resistance to hormonal breast cancer in an individual, comprising the steps of optionally obtaining a sample from a breast of the individual or biopsy samples from breast cancer metastases, such as metastases to the axillary lymph nodes or distant sites, wherein the sample comprises a cell having an estrogen receptor alpha nucleic acid sequence or amino acid sequence; and assaying the nucleic acid sequence for an A908G mutation or the corresponding K303R mutation in the amino acid sequence (or both), wherein the presence of the mutation in the nucleic acid (or amino acid) sequence indicates the individual is resistant to or will become resistant to or is at high risk to develop resistance to hormonal breast cancer therapy.

In an additional embodiment of the present invention there is a method of detecting sensitivity to breast cancer therapy in an individual, comprising the steps of optionally obtaining a sample from a breast of the individual or biopsy samples from breast cancer metastases, such as metastases to the axillary lymph nodes or distant sites, wherein the sample comprises a cell having an estrogen receptor alpha nucleic acid sequence or amino acid sequence; and assaying the nucleic acid sequence for an A908G mutation or the corresponding K303R mutation in the amino acid sequence (or both), wherein the presence of the mutation in the nucleic acid (or amino acid) sequence indicates the individual is sensitive to breast cancer chemotherapy.

In another embodiment of the present invention there is a method of diagnosing breast cancer therapy resistance in an individual, comprising the steps of obtaining a sample from a breast of the individual or biopsy samples from breast cancer metastases, such as metastases to the axillary lymph nodes or distant sites; manipulating (such as dissecting, although the mutation can be identified in whole samples) the sample to differentiate a cell suspected of being cancerous from a noncancerous cell; and assaying the cell suspected of being cancerous for an A908G mutation in an estrogen receptor alpha nucleic acid sequence and/or at the corresponding 303 amino acid of the estrogen receptor alpha amino acid sequence, wherein the presence of the mutation in the nucleic acid sequence indicates the individual has resistance to breast cancer therapy, is susceptible to developing resistance to breast cancer therapy, is at high risk for developing resistance to breast cancer therapy, or will have resistance to breast cancer therapy. In a specific embodiment, the dissection step comprises removal of the cells suspected of being cancerous from the sample by manual manipulation or by laser capture microdissection. In a specific embodiment, the sample is obtained by biopsy. In another specific embodiment, the assaying step is selected from the group consisting of sequencing, single stranded conformation polymorphism, mismatch oligonucleotide mutation detection, and a combination thereof. In an additional specific embodiment, the assaying step is by antibody detection with antibodies to the A908G mutation of the estrogen receptor alpha nucleic acid sequence or is by antibody detection with antibodies to an acetylated estrogen receptor alpha amino acid sequence.

In another embodiment of the present invention there is a kit for identifying resistance to hormonal breast cancer therapy and/or sensitivity to breast cancer chemotherapy comprising reagents suitable for detecting an A908G mutation in an estrogen receptor alpha nucleic acid sequence and/or for detecting a K303R mutation in an estrogen receptor alpha amino acid sequence. In particular embodiments, the kit comprises at least one primer to conduct PCR amplification of the mutation, and/or at least one primer suitable for primer extension such that the primer extension identifies the mutation. The mutation can be detected using standard sequencing techniques, or mass spectroscopy. In specific embodiments, the mutation can be detected by primer extension, sequencing, single stranded conformation polymorphism, mismatch oligonucleotide mutation detection, mass spectroscopy, DNA microarray, HPLC, microarray, SNP PCR genotyping, or a combination thereof, for example. Other exemplary modes of mutation detection utilizing electrophoresis can also be employed, for example. In particular embodiments, the kit comprises at least one primer selected from the group consisting of SEQ ID NO:15, SEQ ID NO:16, SEQ ID NO:17, and SEQ ID NO:18. In one embodiment, the primers are extendable. In an alternative embodiment, the primers are nonextendable. In certain aspects, the kit comprises antibodies that immunologically react with an acetylated estrogen receptor alpha amino acid sequence, or an antigenic fragment thereof.

In another embodiment of the present invention there is a monoclonal antibody that binds immunologically to a mutated estrogen receptor alpha amino acid sequence, or an antigenic fragment thereof. The mutated estrogen receptor alpha amino acid sequence may be acetylated, in some cases.

In another embodiment of the present invention there is a monoclonal antibody that binds immunologically to an A908G mutation in an estrogen receptor alpha nucleic acid sequence.

In an alternative embodiment of the present invention, a cell comprising the mutation replicates faster than a cell that does not comprise the mutation. In specific aspect of this alternative embodiment, cells comprising the mutation are more sensitive to breast cancer chemotherapy than cells that do not comprise the mutation.

In one embodiment, there is a method of identifying a cancer in an individual that is susceptible to becoming resistant to hormonal breast cancer therapy, will become resistant to hormonal breast cancer therapy, or that is resistant to hormonal breast cancer therapy, comprising the step of assaying a sample from the individual for an A908G mutation in estrogen receptor alpha (ERα) sequence, wherein the presence of said mutation in said nucleic acid sequence indicates said individual is susceptible to becoming resistant to hormonal breast cancer therapy, will become resistant to hormonal breast cancer therapy, or is resistant to hormonal breast cancer therapy. In specific embodiments, the hormonal breast cancer therapy comprises one or more antiestrogens or one or more aromatase inhibitors. In a particular embodiment, the method further comprises providing an alternative breast cancer therapy to the individual. In a particular case, a sample is obtained by biopsy.

In specific embodiments of the invention, the assaying step comprises primer extension, sequencing, single stranded conformation polymorphism, mismatch oligonucleotide mutation detection, mass spectroscopy, DNA microarray, HPLC, microarray, SNP PCR genotyping, or a combination thereof. In another specific embodiment, the assaying step is by antibody detection with antibodies to the A908G mutation of the estrogen receptor alpha nucleic acid sequence or is by antibody detection with antibodies to a corresponding estrogen receptor alpha K303R amino acid sequence. In particular cases, the antibodies to the corresponding estrogen receptor alpha K303R amino acid sequence immunologically recognize a modified K303R amino acid sequence. In a specific case, the modified K303R amino acid sequence is acetylated.

In one embodiment of the invention, there is a peptide comprising the estrogen receptor alpha K303 amino acid. In a specific embodiment, the peptide is no more than 20 amino acids long. In an additional specific embodiment, the peptide is no less than 5-6 amino acids long. In a particular aspect, the peptide is from 5-20 amino acids in length. In a further embodiment, there is a mutation at the estrogen receptor alpha S305 amino acid. In another case, the peptide is capable of being phosphorylated at the estrogen receptor alpha S305 amino acid. In a specific embodiment, the peptide is not capable of being phosphorylated at the estrogen receptor alpha S305 amino acid. In an additional embodiment, the estrogen receptor alpha S305 amino acid is acetylated, sumoylated, or methylated.

In an additional embodiment, there is a kit for diagnosing susceptibility or presence of resistance to a hormonal breast cancer therapy in an individual, comprising one or more reagents for detecting an A908G mutation in an estrogen receptor alpha nucleic acid sequence and an alternative breast cancer therapy.

In one embodiment, there is a method of identifying a cancer in an individual that is sensitive to breast cancer chemotherapy, comprising the step of assaying a sample from the individual for an A908G mutation in estrogen receptor alpha (ERα) sequence, wherein the presence of said mutation in said nucleic acid sequence indicates said individual is sensitive to breast cancer chemotherapy.

In one embodiment, there is a method of determining a treatment regimen for an individual suspected of having breast cancer, comprising the steps of assaying a sample from the individual for an A908G mutation in estrogen receptor alpha (ERα) sequence, wherein the presence of the mutation in said nucleic acid sequence indicates the individual is susceptible to becoming resistant to hormonal breast cancer therapy, will become resistant to hormonal breast cancer therapy, or is resistant to hormonal breast cancer therapy; and determining a treatment regimen for the individual based on the outcome of said assaying step. In specific embodiments, the treatment regimen comprises an alternative to hormonal breast cancer therapy.

In one embodiment, there is a method of determining a treatment regimen for an individual suspected of having breast cancer, comprising the steps of assaying a sample from the individual for an A908G mutation in estrogen receptor alpha (ERα) sequence, wherein the presence of said mutation in said nucleic acid sequence indicates said individual is susceptible to becoming resistant to hormonal breast cancer therapy, will become resistant to hormonal breast cancer therapy, or is resistant to hormonal breast cancer therapy; and determining a treatment regimen for the individual based on the outcome of said assaying step. In a specific embodiment, the treatment regimen comprises an alternative to hormonal breast cancer therapy, such as therapy selected from the group consisting of targeted therapy to other molecular alterations in the breast cancer, targeted therapy with signal transduction inhibitors, fulvestrant, chemotherapy, or a steroidal aromatase inhibitor. In a particular case, the targeted therapy to other molecular alterations in the breast cancer targets HER2, EGFR, IGF1R, PI3K, or AKT. In a certain aspect, the targeted therapy with signal transduction inhibitors targets PI3K or AKT. In a specific embodiment, the steroidal aromatase inhibitor comprises exemestane.

In an additional embodiment there is a method of determining response to an aromatase inhibitor in a breast tumor comprising the step of assaying an A908G mutation in estrogen receptor alpha (ERα) sequence from cells from the tumor, wherein when the cells comprise the mutation the breast tumor is or will become resistant to the aromatase inhibitor.

Other and further objects, features, and advantages would be apparent and eventually more readily understood by reading the following specification and be reference to the accompanying drawings forming a part thereof, or any examples of the presently preferred embodiments of the invention given for the purpose of the disclosure.

### BRIEF DESCRIPTION OF THE FIGURES

FIGS. 1A-1C show that K303R ERα mutation generates an estrogen hypersensitive phenotype in vitro and in vivo.

FIGS. 2A-2C show that the K303R ERα mutation confers resistance to the aromatase inhibitor anastrazole (Ana).

FIGS. 3A-3C demonstrate that the PI-3K/Akt pathway is involved in K303R-associated AI^{R} phenotype.

FIGS. 4A-4E demonstrate that K303R AromCells showed an altered apoptotic and proliferative response.

FIG. 5 shows YFP-ERα expression in stably transfected MCF-7 cells.

FIGS. 6A-6B demonstrate that MCF-7 K303R ERα-expressing cells show altered sensitivity to tamoxifen.

FIGS. 7A-7C show that growth factor receptor signaling is amplified in MCF-7 K303R ERα-expressing cells.

FIG. 8 shows that AP-1 activity induced by growth factor in K303R ERα-expressing cells.

FIGS. 9A-9B demonstrate that phosphorylation status of serine 305 in the K303R ERα mutant may be involved in enhancement of growth factor signaling.

FIG. 10 illustrates an exemplary model for growth factor crosstalk with the K303R ERα mutant receptor.

FIGS. 11A-11B show E2 and Tam effects on large-scale chromatin structure in PRL-Hela cells. Cells transiently expressing YFP-ERα WT (WT) or the YFP-K303R ERα (K303R) mutation were pretreated with forskolin (FSK) for 15′ minutes and then were treated with E2 (a) or Tam (b) at different doses for 30 minutes. After fixing and counter-staining with DAPI, cells were imaged and array size was quantified using HTM as described herein.

FIG. 12 shows estrogen-induced signalling in MCF-7 WT-ERαL and MCF-7 K303R ERα-expressing cells, wherein MCF-7 parental, MCF-7 WT, and MCF-7 K303R-1 cells were serum-starved for 48 h, and then treated with or without 1 nM E2 for 24 hours before lysis. Equal amounts of total cellular extract were analyzed for progesterone receptor (PR-A and PR-B) levels by Western blotting.

FIG. 13 shows that heregulin treatment reduced the ability of tamoxifen to inhibit anchorage-independent growth of MCF-7K303R cells, wherein MCF-7 WT-P and MCF-7 K303R-P pool of transfected and overexpressing cells were plated in soft agar and then untreated or treated with heregulin 2 ng/ml (H) in the presence or absence of Herceptin (10 μg/ml). *p=0.0002 vs control (C); **p=0.03 vs heregulin (H) treated cells.

FIGS. 14A-14D show that K303R ERα mutation confers resistance to anastrozole. A, MTT growth assays in cells treated with vehicle, E₂ (1 nmol/L), AD (10 nmol/L), and/or anastrozole (Ana 100 nmol/L, 1 μmol/L, 10 μmol/L). Cell proliferation is expressed as fold change relative to vehicle-treated cells. Data are representative of three independent experiments, performed in quadruplicate. Columns, mean; bars, SD (**, P<0.005; n.s., nonsignificant AD versus Ana +AD). ^{B}, anchorage-independent growth assay in cells treated with vehicle, E2 (1 nmol/L), AD (10 nmol/L) ±Ana (1 μmol/L). Bars, SD (*, P=0.01; n.s., nonsignificant AD versus Ana +AD). C and D, anchorage-independent growth assay in MCF-7 Arom P(C) and CHO P (D) pools treated with vehicle, AD (10 nmol/L) ±Ana (1 μmol/L). Bars, SD (**, P<0.01; n.s., nonsignificant AD versus Ana +AD). C and D, ERα and Rho GDIα (top). Numbers below blots represent aromatase activity.

FIGS. 15A-15F show that mutant cells exhibited constitutive pAkt. A to F, immunoblotting showing phosphorylated Akt (pAkt), Akt, and Rho GDIα in cells treated with vehicle or AD 1, 10, 25, and 50 nmol/L (1 h, A), or AD 10, 30, 60, and 120 min (10 nmol/L, B), or heregulin 2 ng/mL (H 5 min, B); in xenograft tumors extracts (C, numbers below blots represent the average fold change in pAkt levels of K303R Arom 1 samples versus MCF-7 Arom 1 samples); in cells treated with vehicle, AD 10 nmol/L ±Ana 1 Amol/L (1 h, D); in cells treated with vehicle and ICI 1 Amol/L for 1, 5, 10, and 30 min (E) or 1, 3, 24, and 48 h (F). Quantitative analysis is the fold difference in pAkt/Akt/Rho GDIα ratio relative to vehicle-treated MCF-7 Arom 1 cells.

FIGS. 16A-16E show that mutant cells showed increased PI3K/Akt activation. A, lysates from CHO cells transiently transfected with WT or K303R ERα were immunoprecipitated (IP) with anti-p85 or anti-ERα and immunoblotted for ERα or p85. Input, whole-cell lysates. B and C, in vitro PI3K activity of p85 immunoprecipitates from CHO cells transiently transfected with YFP-WT or YFP-K303R ERα, and CHO WT P and CHO K303R P pools (B) or MCF-7 Arom 1 and K303R Arom 1 cells treated with vehicle or LY294002 (LY 10 μmol/L) for 45 min (C). The enzyme activity was expressed as amounts of PIP3 (in picomoles) produced by cells. Columns, means from triplicate readings in two experiments; bars, SD (*, P<0.05; **, P=0.001). B and C, p85 expression in immunoprecipitates (top). D, immunoblotting of pAkt, Akt, phosphorylated Ser167-ERα (pS167), YFP-ERα, and Rho GDIα in CHO cells transiently transfected with YFP-WT or YFP-K303R ERα, or CHO WT P and CHO K303R P pools. E, CHO cells were transiently transfected with YFP-WT or YFP-K303R ERα plus an ERE-luciferase reporter, and treated with vehicle, LY (10 μmol/L), or ICI (1 Amol/L). Luciferase activities were normalized to β-galactosidase for relative luciferase units. Data are representative of three independent experiments, performed in triplicate. Columns, mean; bars, SD (***, P=0.0008 versus vehicle). ERα and Rho GDIα expression was determined by immunoblotting (right).

FIGS. 17A-17E show altered apoptotic response in mutant cells. A to C, immunoblotting showing PARP and Rho GDIα in cells treated with vehicle, E₂ 1 nmol/L (24 h), AD 10 nmol/L (12, 24, and 48 h, A), or AD 10 nmol/L and AD +Ana 1 μmol/L (24 h, B); Bcl-2, Bax, and Rho GDIα in cells treated with vehicle, AD 10 nmol/L ±Ana 1 μmol/L (24 h, C). D, quantitative analysis is the fold difference in Bcl-2/Bax ratio relative to vehicle-treated MCF-7 Arom 1 cells. E, ELISA cell detection assay in cells treated with AD 10 nmol/L ±Ana 1 μmol/L. Columns, mean; bars, SD (*, P<0.05, **, P<0.005, n.s., nonsignificant AD versus Ana +AD).

FIGS. 18A-18E show inhibition of PI3K/Akt pathway reversed AI^{R}. A, immunoblotting showing phosphorylated Akt and Akt in cells treated with vehicle, LY 10 Amol/L or PI-103 (1, 5, and 10 μmol/L) or Akti1/2 (1, 5, and 10 μmol/L) for 30 min. B to D, anchorage-independent growth assay in cells treated with vehicle, AD 10 nmol/L, Ana 1 μmol/L ±LY 10 μmol/L (B) or Akti1/2 1 μmol/L (C) or PD98059 (PD 10 μmol/L, D) or ICI 1 μmol/L (D). Bars, SD (***, P<0.0005 LY, Akti1/2 and ICI-treated cells versus control cells and cells treated with AD or Ana +AD). E, cell death detection assay in cells treated with AD 10 nmol/L F Ana 1 μmol/L F LY 10 μmol/L. Columns, mean; bars, SD (*, P<0.05 versus AD in MCF-7 Arom 1 and AD and Ana+AD in K303R Arom 1).

### DETAILED DESCRIPTION OF THE INVENTION

The present application incorporates by reference herein in their entirety the following: U.S. Pat. No. 6,821,732; U.S. Pat. No. 7,429,650; and U.S. Pat. No. 7,419,785.

It will be readily apparent to one skilled in the art that various embodiments and modifications may be made in the invention disclosed herein without departing from the scope and spirit of the invention.

Unless defined otherwise, technical and scientific terms used herein have the same meaning as commonly understood by one of ordinary skill in the art to which this invention belongs. For purposes of the present invention, the following terms are defined below.

As used herein, the use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

### I. DEFINITIONS

The term “A908G mutation” as used herein is defined as an adenine (A)-to-guanine (G) base pair transition at nucleotide position 908 in an estrogen receptor alpha nucleic acid sequence, relative to the first nucleotide of the first codon of the translated amino acid sequence. A skilled artisan recognizes that multiple estrogen receptor alpha nucleic acid sequences exist which are, for example, alternative splice variants. Thus, there are some estrogen receptor alpha nucleic acid sequences of different sizes, and the A908G mutation which is present at nucleotide (nt) 908 in the full-length mutated sequence may no longer be at position 908 in a variant sequence. However, a skilled artisan can readily identify the equivalent or analogous sequence in these variants by sequence homology and comparison, and/or by analyzing locations, arrangements or relationships of splicing manipulations. Thus, an estrogen receptor alpha nucleic acid sequence which contains the indicated mutation yet is a variant, such as an alternatively spliced form of the sequence, is still within the scope of the present invention.

The term “agonist” as used herein is defined as a compound or composition that promotes, facilitates, allows, induces, or otherwise assists, activates or increases the function of the estrogen receptor alpha K303R polypeptide.

The term “antagonist” as used herein is defined as a compound or composition that inhibits, stops, deters, impedes, delays, or otherwise prevents the activity and functioning of the estrogen receptor alpha K303R polypeptide.

The term “biopsy” as used herein is defined as removal of a tissue from a breast, or from breast tumors that have metastasized to other sites in the body, for the purpose of examination, such as to establish diagnosis. Examples of types of biopsies include by application of suction, such as through a needle attached to a syringe; by instrumental removal of a fragment of tissue; by removal with appropriate instruments through an endoscope; by surgical excision, such as of the whole lesion; and the like.

The term “breast cancer” as used herein is defined as cancer that originates in the breast. In a specific embodiment, the breast cancer spreads to other organs, such as lymph nodes. In a specific embodiment, the breast cancer is invasive and may be metastatic.

The term “cancer” as used herein is defined as a new growth of tissue comprising uncontrolled and progressive multiplication. In a specific embodiment, upon a natural course the cancer is fatal. In specific embodiments, the cancer is invasive, metastatic, and/or anaplastic (loss of differentiation and of orientation to one another and to their axial framework).

The term “chemotherapy” as used herein refers to any agent, chemical, or biologic that is used to treat breast cancer growth

The term “hormonal therapy” as used herein refers to agents that target estrogen receptor.

The term “invasive” as used herein refers to cells that have the ability to infiltrate surrounding tissue. In a specific embodiment, the infiltration results in destruction of the surrounding tissue. In another specific embodiment, the cells are cancer cells. In a preferred embodiment, the cells are breast cancer cells, and the cancer spreads out of a duct into surrounding breast epithelium. In a specific embodiment, “metastatic” breast cancer is within the scope of “invasive.”

The term “K303R substitution” as used herein is defined as the amino acid substitution that results from the A908G mutation in estrogen receptor alpha nucleic acid sequence. The term “Lys303Arg substitution” is used herein interchangeably. A skilled artisan recognizes that multiple estrogen receptor alpha amino acid sequences exist which are, for example, alternative splice variants. Thus, there are some estrogen receptor alpha amino acid sequences of different sizes, and the K303R substitution which is present in the full-length mutated sequence may no longer be at position 303 in the variant sequence. However, a skilled artisan can readily identify the equivalent or analogous sequence in these variants by sequence homology and comparison, and/or by analyzing locations, arrangements or relationships of splicing manipulations. Thus, an estrogen receptor alpha amino acid sequence which contains the indicated mutation yet is a variant, such as an alternatively spliced form of the sequence, is still within the scope of the present invention.

The term “laser capture microdissection” as used herein is defined as the use of an infrared (IR) laser beam to remove a desired cell from a nondesired cell. In preferred embodiments, the desired cell is a cancer cell and the nondesired cell is a normal cell. In another preferred embodiment, the cancer cell is a breast cancer cell.

The term “manual manipulation” as used herein is defined as the selective removal of a desired cell or cells from a nondesired cell or cells by hand. In preferred embodiments, the desired cell is a cancer cell and the nondesired cell is a normal cell. In another preferred embodiment, the cancer cell is a breast cancer cell.

The term “metastatic” as used herein is defined as the transfer of cancer cells from one organ or part to another not directly connected with it. In a specific embodiment, breast cancer cells spread to another organ or body part, such as lymph nodes.

The term “premalignant lesion” as used herein is defined as a collection of cells in a breast with histopathological characteristics which suggest at least one of the cells has an increased risk of becoming breast cancer. A skilled artisan recognizes that the most important premalignant lesions recognized today include unfolded lobules (UL; other names: blunt duct adenosis, columnar alteration of lobules), usual ductal hyperplasia (UDH; other names: proliferative disease without atypia, epitheliosis, papillomatosis, benign proliferative disease), atypical ductal hyperplasia (ADH), atypical lobular hyperplasia (ALH), ductal carcinoma in situ (DCIS), and lobular carcinoma in situ (LCIS). Other lesions which may have premalignant potential include intraductal papillomas, sclerosisng adenosis, and fibroadenomas (especially atypical fibroadenomas). In a specific embodiment, the collection of cells is a lump, tumor, mass, bump, bulge, swelling, and the like. Other terms in the art which are interchangeable with “premalignant lesion” include premalignant hyperplasia, premalignant neoplasia, and the like.

The term “resistance to breast cancer therapy” as used herein refers to a condition when an individual's tumor either inherently (de novo resistance) do not respond to breast cancer treatment or when an individual's cancer develops the ability to continue to grow in spite of treatment (acquired resistance).

The term “sample from a breast” as used herein is defined as a specimen from any part or tissue of a breast. A skilled artisan recognizes that the sample may be obtained by any method, such as biopsy. In a specific embodiment the sample is obtained by nipple aspirate (see, for example, Sauter et al. (1997)). In another specific embodiment, the sample is from hyperplastic or malignant breast epithelium. In a specific embodiment, the sample is from the epithelium. In another specific embodiment, the sample is from a premalignant lesion. A skilled artisan recognizes that within the scope of the present invention is the embodiment wherein a normal, or benign, sample, such as from an epithelium, is obtained for risk screening.

In an alternative embodiment, a sample may come from an organ or tissue that is not the breast but that is a sample from distant breast cancer that has metastasized thereto.

### II. THE PRESENT INVENTION

The present invention concerns resistance to hormonal breast cancer therapy or sensitivity to breast cancer chemotherapy. In particular cases, the present invention concerns resistance to hormonal breast cancer therapy, identification of hormonal resistance to breast cancer therapy, and/or therapy for the individual resistant to the hormonal breast cancer therapy. The individual may be known to have breast cancer, may not be known to have breast cancer, may be suspected of having breast cancer (for example, the individual has a lump in one or both breasts), or may be at risk for having breast cancer (for example, the individual has a family or personal history of breast cancer; has a lump in the breast, is on the birth control pill; has genetic predisposition (such as mutation in one or more of BRCA1, BRCA2, ATM, CHEK2, p53, or PTEN, for example); has history of breast hyperplasia; began menses early (before the age of 12); began menopause late (older than 55); has or had postmenopausal hormone therapy; were nulliparous, or had children after the age of 30; had a BMI above the 80 percentile; and/or were greater than 65 years of age). The individual may be female or male and may be of any race or age. In specific embodiments, the individual is a female over the age of 20, 25, 30, 35, 40, 45, 50, 55, 60, 65, 70, 75, 80, or 85. The type of breast cancer having one or more cells that have become resistant to the cancer may be of any kind, including in situ breast cancer (for example, ductal carcinoma in situ); invasive breast cancer (for example, invasive ductal carcinoma (including tubular, mucinous, medullary and papillary) or invasive lobular carcinoma); inflammatory breast cancer; phyllodes tumor; angiosarcoma; osteosarcoma; metaplastic breast cancer; adenoid cystic carcinoma; or Paget' s disease of the breast. The breast cancer may be HER2⁺ or HER2⁻. It may be progesterone receptor (PR)⁺ or PR⁻. In particular embodiments, the cancer is ER⁺. The breast cancer to which the therapy has become resistant may be metastatic, in certain instances. The breast cancer cells may be grade 1, grade 2, or grade 3. The type of breast cancer may be determined by any suitable method(s), including immunohystochemistry and/or FISH, and so forth.

As discussed above, estrogens play a crucial role in regulating breast tumor growth, which in certain cases is the rationale for the use of antiestrogens, such as tamoxifen (Tam), in women with estrogen receptor (ER)-α-positive breast cancer; however resistance is a major clinical problem. Altered growth factor signaling to the ERα pathway has been shown to be associated with the development of clinical resistance. The inventor and others previously identified a mutation at nucleotide 908 of ERα (A908G) in premalignant hyperplasias and invasive breast cancers. This mutation replaces arginine for lysine at residue 303 (K303R ERα), and confers the ability for enhanced growth in low physiological levels of estrogen. To demonstrate that the mutation at this site plays a role in resistance, the inventor generated MCF-7 breast cancer cells stably transfected with either wild-type (WT) or K303R mutant ERα. Additive effects of various growth factors (heregulin, epidermal growth factor [EGF], and insulin growth factor [IGF]) were tested in the absence or presence of Tam on cell growth in these cells. The mutation alters Tam response, and growth factor stimulation converted Tam into an agonist in mutant-expressing cells. Tam was less efficient at reducing estrogen-stimulated growth of mutant expressing cells in soft agar assays as well. Tam was also unable to block heregulin-induced soft agar growth of mutant cells, as compared to MCF-7 WT cells. Mutant cells had increased phosphorylated HER2, Akt, and MAPK levels compared to WT cells. Time course studies demonstrated earlier heregulin-induced phosphorylation of these signalling molecules and c-Src in K303R-expressing cells. To examine the effects of growth factors on estrogen-induced genomic activity, transcriptional assays were performed with an AP-1 luciferase gene reporter. EGF and heregulin induced high AP-1 activity, but only in mutant-expressing cells. In specific embodiments of the invention, the mutation adapts the receptor for enhanced bidirectional cross-talk with growth factor signaling pathways, which then impacts on Tam response. These results indicate that the presence of the A908G (or corresponding K303R) ERα mutation is useful as a predictive biomarker of hormonal response in patients whose tumors exploit ERα cross-talk to evade treatment.

Other embodiments of the invention include resistance to aromatase inhibitors (AI), which are the treatment of choice for steroid receptor positive breast cancer, but resistance to these inhibitors is a problem. The A908G estrogen receptor (ER) a mutation conferred a hypersensitivity to estrogen that was maximally stimulated in response to low physiological levels of hormone. In a specific embodiment of the invention, such a mutant provides a continuous mitogenic stimulus to the breast even during phases of low circulating hormone, such as in postmenopausal women, thus affording a proliferative advantage especially during treatment with AIs. The K303R ERα mutation also renders the receptor a more efficient substrate for phosphorylation at serine (S) 305, which results in enhanced hormone sensitivity for growth. To demonstrate that the mutation is resistant to AIs, the inventor generated MCF-7 parental and MCF-7-K303R-overexpressing cell lines stably transfected with an aromatase expression vector. Cells were stimulated with the aromatase substrate, androstenedione (AD) ±the exemplary AI anastrazole (Ana). Ana decreased AD-stimulated growth of WT vector control cells, but had no effect on AD-stimulated growth of MCF-7-K303R-overexpressing cells using in vitro MTT assays. Furthermore, the basal non-stimulated colony number of the mutant cells was increased 10-fold in soft agar assays, and Ana was unable to inhibit the formation of mutant colonies. Aromatase activity of both cells was reduced by 95% with Ana treatment, indicating that resistance cannot be explained by an intrinsic insensitivity of the aromatase enzyme itself to Ana. The mutation generated a new site for AKT phosphorylation at Ser305. Constitutively increased levels of phospho-AKT in mutant cells were observed. K303R mutant-overexpressing cells exhibited increased levels of cyclin D1, and decreased levels of the CDK inhibitor p21 during AI treatment. Accordingly, apoptosis was altered with decreased Bax levels and PARP cleavage, increased levels of phospho-BAD, and bcl-2. These cumulative results indicate that the mutation indeed confers resistance to an AI, and, in specific embodiments of the invention, mechanisms of resistance include cellular strategies to evade apoptosis and enhance proliferation, for example through enhanced reception of downstream signaling by growth factor networks, such as AKT. The results indicate that the mutation is a new predictive marker of response to AIs in mutation-positive breast tumors.

In a specific embodiment, the mutation is sensitive to steroidal aromatase inhibitors but is resistant to nonsteroidal aromatase inhibitors (AIs). In a further specific embodiment, the mutation is resistant to nonsteroidal AIs like anastrazole and letrozole (commonly used in patients), but it is sensitive to steroidal AI exemestane, for example.

A skilled artisan recognizes the existence of a variety of inherited, or somatically acquired, variations in the DNA of the estrogen receptor alpha gene in cells in a breast sample, which, in the latter case, may differ in a mixture of normal and neoplastic cells. As demonstrated in the Examples herein, those cells having DNA that contain an A908G mutation in the estrogen receptor alpha nucleic acid sequence are or will become resistant to therapy. The present invention is directed to methods and compositions related to detection of the A908G mutation or the corresponding K303R mutation in relation to resistance to breast cancer therapy.

In an embodiment of the present invention there is an isolated estrogen receptor alpha nucleic acid sequence comprising an A908G mutation.

In another embodiment of the present invention there is an isolated estrogen receptor alpha amino acid sequence comprising a K303R substitution.

In an additional embodiment of the present invention there is a method of detecting susceptibility to development of resistance to breast cancer therapy in an individual, comprising the steps of obtaining a sample from a breast of the individual, wherein the sample comprises a cell having an estrogen receptor alpha nucleic acid sequence; and assaying the nucleic acid sequence for an A908G mutation, wherein the presence of the mutation in the nucleic acid sequence indicates the individual has or will become resistant to breast cancer therapy or detects susceptibility of the cell to develop resistance to breast cancer therapy.

In an additional specific embodiment of the present invention an assaying step is by antibody detection with antibodies to the A908G mutation of the estrogen receptor alpha nucleic acid sequence or by antibody detection with antibodies to an estrogen receptor alpha amino acid sequence, including an acetylated estrogen receptor alpha amino acid sequence, for example.

In another embodiment of the present invention there is a method of diagnosing breast cancer therapy resistance in an individual, comprising the steps of assaying for an A908G mutation, wherein the presence of the mutation in the nucleic acid sequence indicates the individual has or will develop resistance to breast cancer therapy.

In another embodiment of the present invention there is a method of diagnosing breast cancer therapy resistance in an individual, comprising the steps of optionally obtaining a sample from a breast of the individual; dissecting the sample to differentiate a cell suspected of being cancerous from a noncancerous cell; and assaying the cell suspected of being cancerous for an A908G mutation in an estrogen receptor alpha nucleic acid sequence, wherein the presence of the mutation in the nucleic acid sequence indicates the individual has breast cancer. In a specific embodiment, the dissection step comprises removal of the cells suspected of being cancerous from the sample by manual manipulation or by laser capture microdissection. In a specific embodiment, the sample is obtained by biopsy. In another specific embodiment, the assaying step is selected from the group consisting of primer extension, sequencing, single stranded conformation polymorphism, mismatch oligonucleotide mutation detection, and a combination thereof. In an additional specific embodiment, the assaying step is by antibody detection with antibodies to the A908G mutation of the estrogen receptor alpha nucleic acid sequence or is by antibody detection with antibodies to an acetylated estrogen receptor alpha amino acid sequence. In a specific embodiment, the mutation is detected by SNP analysis, using standard methods in the art. Some methods use extendable primers for incorporating radiolabeled nucleotides, which can then be detected by fluorescence or resonance. For example, PerkinElmer™ (Shelton, Conn.) has the AcycloPrime™ fluorescence polarization SNP detection system that utilizes terminator labeled nucleotides to facilitate detection of the SNP upon fluorescence polarization. Also, Applied Biosystems (Foster City, Calif.) has the ABI PRISM® turbo TaqMan® probes for genotyping by allelic detection which utilizes fluorescent dyes, such as VIC™, and TET and 6-FAM, for detection. In a specific embodiment, the thymidine residues of the probes are replaced with 5-propyne-2′-deoxyuridine, which increases the Tₘ of these probes by approximately 1° C. per substitution and facilitates design of a shorter probe for greater accuracy. Mass spectroscopy may also be employed to detect the mutation.

In another embodiment of the present invention there is a kit for diagnosing an A908G mutation in an estrogen receptor alpha nucleic acid sequence, comprising at least one primer suitable for use in primer extension to detect the mutation. In a specific embodiment, SEQ ID NO:15, SEQ ID NO:16, SEQ ID NO:17, and/or SEQ ID NO:18 are employed in the kit. In a specific embodiment, the kit contains primers that are extendable. In an alternative specific embodiment, the kit contains primers that are nonextendable.

In another embodiment of the present invention there is a monoclonal antibody that binds immunologically to an acetylated estrogen receptor alpha amino acid sequence, or an antigenic fragment thereof.

In another embodiment of the present invention there is a monoclonal antibody that binds immunologically to an A908G mutation in an estrogen receptor alpha nucleic acid sequence or the corresponding K303R mutation in the estrogen receptor alpha amino acid sequence.

### III. DETECTION OF THE MUTATION

A skilled artisan recognizes that there are a variety of methods to detect a mutation in a nucleic acid sequence. In some cases, the mutation is assayed by primer extension, sequencing, single stranded conformation polymorphism, mismatch oligonucleotide mutation detection, mass spectroscopy, DNA microarray, HPLC, microarray, SNP PCR genotyping, or a combination thereof. In particular embodiments, the mutation is assayed by utilizing methods as reported in Herynk et al. (2007), for example, which is incorporated by reference herein in its entirety. As described therein, primer extension analysis is employed to detect the mutation. In certain embodiments of the invention, mass spectroscopy is employed to detect the mutation.

Methods regarding allele-specific probes for analyzing particular nucleotide sequences are described by e.g., Saiki et al., Nature 324, 163-166 (1986); Dattagupta, EP 235,726 (U.S. 836,378 (Mar. 5, 1986); U.S. 943,006 (Dec. 29, 1986)); Saiki, WO 89/11548 (U.S. 197,000 (May 20, 1988); U.S. 347,495 (May 4, 1989)). Allele-specific probes are typically used in pairs. One member of the pair shows perfect complementarity to a wildtype allele and the other members to a variant allele. In idealized hybridization conditions to a homozygous target, such a pair shows an essentially binary response. That is, one member of the pair hybridizes and the other does not. An allele-specific primer hybridizes to a site on target DNA overlapping the particular site in question and primes amplification of an allelic form to which the primer exhibits perfect complementarily (Gibbs, 1989). This primer is used in conjunction with a second primer which hybridizes at a distal site. Amplification proceeds from the two primers leading to a detectable product signifying the particular allelic form is present. A control is usually performed with a second pair of primers, one of which shows a single base mismatch at the polymorphic site and the other of which exhibits perfect complementarily to a distal site. The single-base mismatch impairs amplification and little, if any, amplification product is generated.

Particular nucleic acid sites can also be identified by hybridization to oligonucleotide arrays. An example is described in WO 95/11995, which includes arrays having four probe sets. A first probe set includes overlapping probes spanning a region of interest in a reference sequence. Each probe in the first probe set has an interrogation position that corresponds to a nucleotide in the reference sequence. That is, the interrogation position is aligned with the corresponding nucleotide in the reference sequence when the probe and reference sequence are aligned to maximize complementarily between the two. For each probe in the first set, there are three corresponding probes from three additional probe sets. Thus, there are four probes corresponding to each nucleotide in the reference sequence. The probes from the three additional probe sets are identical to the corresponding probe from the first probe set except at the interrogation position, which occurs in the same position in each of the four corresponding probes from the four probe sets, and is occupied by a different nucleotide in the four probe sets. Such an array is hybridized to a labeled target sequence, which may be the same as the reference sequence, or a variant thereof. The identity of any nucleotide of interest in the target sequence can be determined by comparing the hybridization intensities of the four probes having interrogation positions aligned with that nucleotide. The nucleotide in the target sequence is the complement of the nucleotide occupying the interrogation position of the probe with the highest hybridization intensity.

WO 95/11995 also describes subarrays that are optimized for detection of variant forms of a precharacterized nucleotide site. A subarray contains probes designed to be complementary to a second reference sequence, which can be an allelic variant of the first reference sequence. The second group of probes is designed by the same principles as above except that the probes exhibit complementarity to the second reference sequence. The inclusion of a second group can be particularly useful for analyzing short subsequences of the primary reference sequence in which multiple mutations are expected to occur within a short distance commensurate with the length of the probes (i.e., two or more mutations within 9 to 21 bases).

An additional strategy for detecting a particular nucleotide site uses an array of probes is described in EP 717,113 (U.S. 327,525 (Oct. 21, 1994). In this strategy, an array contains overlapping probes spanning a region of interest in a reference sequence. The array is hybridized to a labeled target sequence, which may be the same as the reference sequence or a variant thereof. If the target sequence is a variant of the reference sequence, probes overlapping the site of variation show reduced hybridization intensity relative to other probes in the array. In arrays in which the probes are arranged in an ordered fashion stepping through the reference sequence (e.g., each successive probe has one fewer 5′ base and one more 3′ base than its predecessor), the loss of hybridization intensity is manifested as a “footprint” of probes approximately centered about the point of variation between the target sequence and reference sequence.

Mundy, C. R. (U.S. Pat. No. 4,656,127), for example, discusses a method for determining the identity of the nucleotide present at a particular site that employs a specialized exonuclease-resistant nucleotide derivative. A primer complementary to the allelic sequence immediately 3′ to the site is permitted to hybridize to a target molecule obtained from a particular animal or human. If the site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the site of the target molecule was complementary to that of the nucleotide derivative used in the reaction. The Mundy method has the advantage that it does not require the determination of large amounts of extraneous sequence data. It has the disadvantages of destroying the amplified target sequences, and unmodified primer and of being extremely sensitive to the rate of polymerase incorporation of the specific exonuclease-resistant nucleotide being used.

Cohen, D. et al. (French Patent 2,650,840 (U.S. Pat. No. 4,420,902 (Dec. 20, 1983)); PCT Appln. No. WO91/02087) discuss a solution-based method for determining the identity of the nucleotide of a particular site. As in the Mundy method of U.S. Pat. No. 4,656,127, a primer is employed that is complementary to allelic sequences immediately 3′ to the site. The method determines the identity of the nucleotide of that site using labeled dideoxynucleotide derivatives, which, if complementary to the nucleotide of the site will become incorporated onto the terminus of the primer.

An alternative method, known as Genetic Bit Analysis or GBA™ is described by Goelet, P. et al. (PCT Appln. No. 92/15712 (U.S. 664,837 (Mar. 5, 1991); U.S. 775,786 (Oct. 11, 1991)). The method of Goelet, P. et al. uses mixtures of labeled terminators and a primer that is complementary to the sequence 3′ to a site in question. The labeled terminator that is incorporated is thus determined by, and complementary to, the nucleotide present in the site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase. It is thus easier to perform, and more accurate than the method discussed by Cohen.

An alternative approach, the “Oligonucleotide Ligation Assay” (“OLA”) (Landegren, U. et al., Science 241:1077-1080 (1988)) has also been described as capable of detecting a nucleotide sequence variation. The OLA protocol uses two oligonucleotides which are designed to be capable of hybridizing to abutting sequences of a single strand of a target. One of the oligonucleotides is biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990). In this method, PCR is used to achieve the exponential amplification of target DNA, which is then detected using OLA. In addition to requiring multiple, and separate, processing steps, one problem associated with such combinations is that they inherit all of the problems associated with PCR and OLA.

Recently, several primer-guided nucleotide incorporation procedures for assaying particular sites in DNA have been described (Komher, J. S. et al., Nucl. Acids. Res. 17:7779-7784 (1989); Sokolov, B. P., Nucl. Acids Res. 18:3671 (1990); Syv anen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)).

### IV. ESTROGEN RECEPTOR ALPHA

Estrogen, mediated through the estrogen receptor (ER), plays a major role in regulating the growth and differentiation of normal breast epithelium (Pike et al., 1993; Henderson et al., 1988). It stimulates cell proliferation and regulates the expression of other genes, including the progesterone receptor (PgR). PgR then mediates the mitogenic effect of progesterone, further stimulating proliferation (Pike et al., 1993; Henderson et al., 1988). Several studies have assessed ER expression in normal breast epithelium and premalignant lesions. Studies of normal terminal duct lobular units (TDLUs) reported that nearly all (over 90%) express ER, but in a minority (averaging about 30%) of cells for all ages combined (Schmitt, 1995; Mohsin et al., 2000; Allegra et al., 1979; Peterson et al., 1986; Ricketts et al., 1991). In premenopausal women, the average proportion of ER-positive cells in TDLUs is somewhat lower (about 20%), and varies with the menstrual cycle, being twice as high during the follicular as the luteal phase (Ricketts et al., 1991). Proliferation in TDLUs peaks during the luteal phase (Potten et al., 1988), suggesting that the normal mitogenic effect of estrogen may be partially delayed or indirect and mediated by downstream interactions such as that between progesterone and PgR. In postmenopausal women, the average proportion of ER-positive cells in TDLUs is relatively high (about 50%) and stable in the absence of hormone replacement therapy (Mohsin et al., 2000). Very little is know about ER expression in ULs, although one preliminary study reported that virtually all expressed the receptor in over 90% of cells (Mohsin et al., 2000). A few studies have evaluated ER in ADH and collectively agreed that nearly all lesions express very high levels in nearly all cells (Schmitt, 1995; Mohsin et al., 2000; Barnes and Masood, 1990). Many studies have evaluated ER in DCIS and, on average, about 75% of all cases expressed the receptor (Mohsin et al., 2000; Zafrani et al., 1994; Albonico et al., 1996; Berardo et al., 1996; Barnes and Masood, 1990; Helin et al., 1989; Giri et al., 1989; Chaudhuri et al., 1993; Poller et al., 1993; Pallis et al., 1992; Leal et al., 1995; Karayiannakis et al., 1996; Bose et al., 1996). Expression varied with histological differentiation, being highest in non-comedo (non-mammary ductal) lesions, where up to 100% showed expression in over 90% of cells, and lowest in comedo lesions, where only about 30% showed expression in a minority of cells. ER was not expressed in about 25% of DCIS and these were predominately high-grade comedo lesions. Over 90% of LCIS expressed high levels of ER in nearly all cells (Fisher et al., 1996; Rudas et al., 1997; Querzoli et al., 1998; Libby et al., 1998; Giri et al., 1989; Pallis et al., 1992; Paertschuk et al., 1990), which is similar in ALH in a specific embodiment.

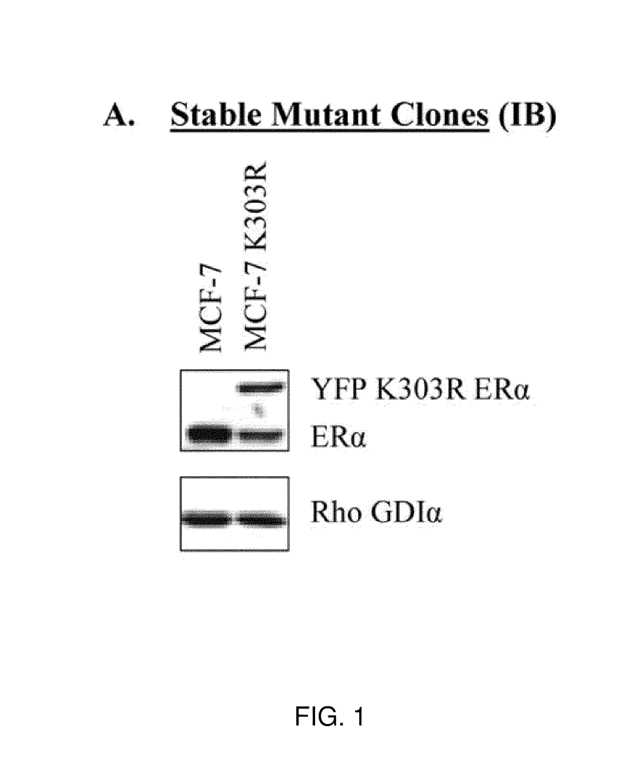

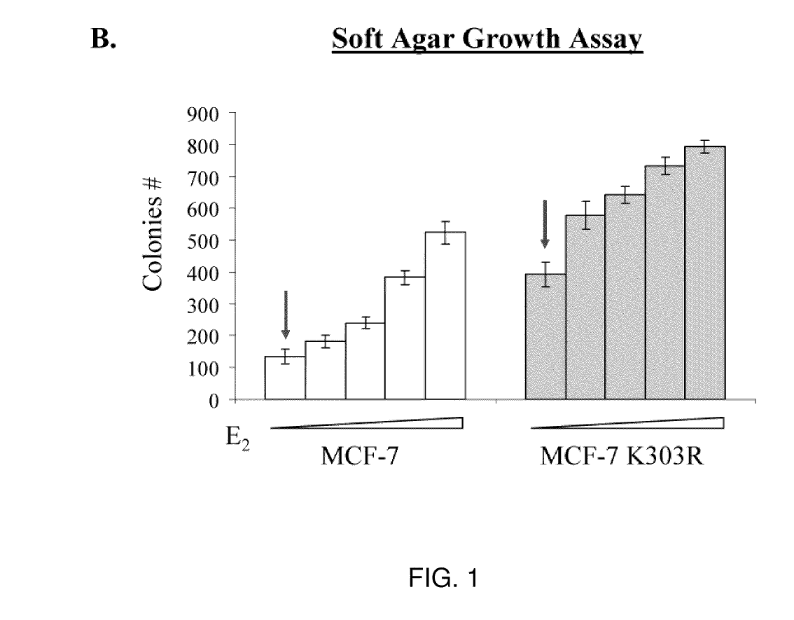

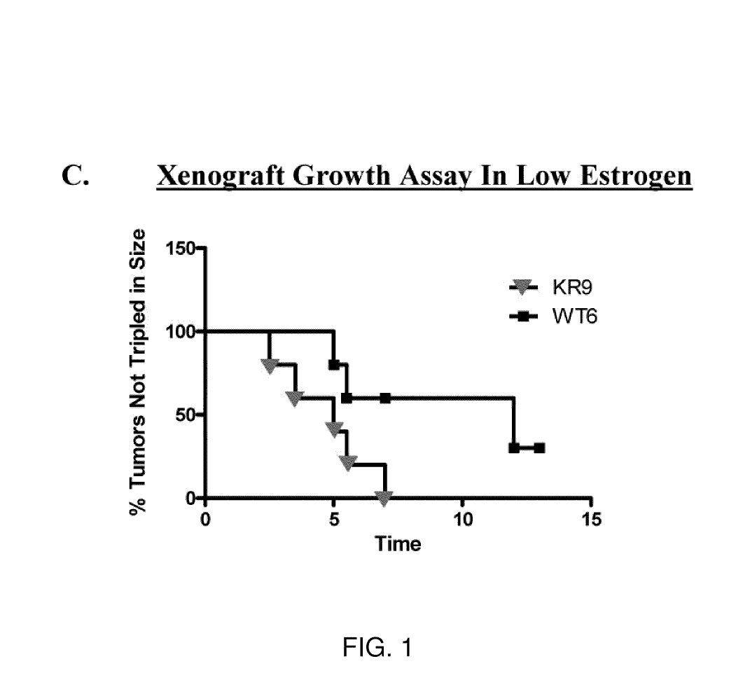

Prolonged estrogen exposure is an important risk factor for developing IBC, perhaps by allowing random genetic alterations to accumulate in normal cells stimulated to proliferate (Henderson et al. 1988), which may also be true for cells in premalignant lesions. The very high levels of ER observed in nearly all premalignant lesions (FIG. 1) may contribute to their increased proliferation relative to normal cells by allowing them to respond more effectively to any level of estrogen, even the low concentrations seen in postmenopausal women (Mohsin et al., 2000). FIG. 1 illustrates examples of typical estrogen receptor expression in premalignant breast lesions as assessed by immunohistochemistry (small dark nuclei are ER-positive cells). Terminal duct lobular units (TDLUs) in premenopausal (pre) women usually contain relatively few ER positive cells. In contrast, the majority of cells in TDLUs of postmenopausal (post) express ER. Most premalignant breast lesions show very high levels of ER in nearly all cells, including unfolded lobules (UIs), atypical ductal hyperplasias (ADHs), low grade “non-comedo” ductal carcinoma in situ (ncDCIS), atypical lobular hyperplasias (ALHs), and lobular carcinoma in situ (LCIS). The only significant exception is high grade “comedo” DCIS (cDCIS), which often show low or no ER expression.

In addition to increased levels of expression, there may be other alterations of ER resulting in increased growth in premalignant lesions. For example, in one recent study (Mohsin et al., 2000), proliferation was measured in TDLUs and premalignant lesions from the same breasts in a large number of patients stratified by menopausal status. Proliferation rates in TDLUs were nearly 3-fold lower in postmenopausal compared to premenopausal women, consistent with the expected mitogenic effect of estrogen and progesterone in normal cells. In contrast, the difference in proliferation in premalignant lesions stratified by menopausal status was less than half that of normal cells, suggesting that the hormonal regulation of proliferation in these lesions, in a specific embodiment, is fundamentally abnormal. It is an object of the present invention to diagnose such an abnormality by identifying an A908G mutation in estrogen receptor alpha nucleic acid sequence or a K303R substitution in the amino acid sequence.

### V. LASER CAPTURE MICRODISSECTION

Developments in gene sequencing and amplification techniques, among others, now allow scientists to extract DNA or RNA from tissue biopsies and cytological smears for pinpoint molecular analysis, such as a point mutation in a nucleic acid sequence. The efficacy of these sophisticated genetic testing methods, however, depends on the purity and precision of the cell populations being analyzed. Simply homogenizing the biopsy sample results in an impure combination of healthy and diseased tissue. Using mechanical tools to manually separate cells of interest from the histologic section is time-consuming and extremely labor-intensive. None of these methods offers the ease, precision and efficiency necessary for modern molecular diagnosis.

The process of laser capture microdis section (LCM) circumvents many problems in the art regarding accuracy, efficiency and purity. A laser beam focally activates a special transfer film which bonds specifically to cells identified and targeted by microscopy within the tissue section. The transfer film with the bonded cells is then lifted off the thin tissue section, leaving all unwanted cells behind (which would contaminate the molecular purity of subsequent analysis). The transparent transfer film is applied to the surface of the tissue section. Under the microscope, the diagnostic pathologist or researcher views the thin tissue section through the glass slide on which it is mounted and chooses microscopic clusters of cells to study. When the cells of choice are in the center of the field of view, the operator pushes a button which activates a near IR laser diode integral with the microscope optics. The pulsed laser beam activates a precise spot on the transfer film immediately above the cells of interest. At this precise location the film melts and fuses with the underlying cells of choice. When the film is removed, the chosen cell(s) are tightly held within the focally expanded polymer, while the rest of the tissue is left behind. This allows multiple homogeneous samples within the tissue section or cytological preparation to be targeted and pooled for extraction of molecules and analysis.

In a commercial system, such as with the instruments and methods of Arcturus (Mountain View, Calif.) (http://www.arctur.com/), the film is permanently bonded to the underside of a transparent vial cap. A mechanical arm precisely positions the transfer surface onto the tissue. The microscope focuses the laser beam to discrete sizes (presently either 30 or 60 micron diameters), delivering precise pulsed doses to the targeted film. Targeted cells are transferred to the cap surface, and the cap is placed directly onto a vial for molecular processing. The size of the targeting pulses is selected by the operator. The cells adherent to the film retain their morphologic features, and the operator can verify that the correct cells have been procured.

Examples of LCM with Breast Tissue include those available at http://www.arctur.com/technology/lcm_examples/ex_breast.html.

Methods regarding the specific preparations and techniques associated with LCM are well known in the art and are provided at the website for Arctur, including: Paraffin-Embedded Tissue, Frozen Tissue, White Blood Cell Cytospin, De-Paraffinization of Tissue Sections, Hematoxylin and Eosin Staining, Immunohistochemical Staining (IHC), Intercalator Dye Staining (Fluorescence), Methyl Green Staining, Nuclear Fast Red Staining, and Toluidine Blue O Staining.

An example of Laser Capture Microdissection steps, particularly for use with Acturus instruments, includes the following:

1. Prepare. Follow routine protocols for preparing a tissue or smear on a standard microscope slide. Apply a Prep Strip™ to flatten the tissue and remove loose debris prior to LCM.

2. Place. Place a CapSure™ HS onto the tissue in the area of interest. The CapSure™ HS is custom designed to keep the transfer film out of contact with the tissue.

3. Capture. Pulse the low power infrared laser. The laser activates the transfer film which then expands down into contact with the tissue. The desired cell(s) adhere to the CapSure™ HS transfer film.

4. Microdissect. Lift the CapSure™ HS film carrier, with the desired cell(s) attached to the film surface. The surrounding tissue remains intact.

5. Extract. Snap the ExtracSure™ onto the CapSure™ HS. The ExtracSure™ is designed to accept low volumes of digestion buffer while sealing out any non-selected material from the captured cells. Pipette the extraction buffer directly into the digestion well of the ExtracSure™. Place a microcentrifuge tube on top.

6. Analyze. Invert the microcentrifuge tube. After centrifuging, the lysate will be at the bottom of the tube. The cell contents, DNA, RNA or protein, are ready for subsequent molecular analysis.

### VI. MISMATCH OLIGONUCLEOTIDE MUTATION DETECTION

A skilled artisan recognizes that one method to identify a point mutation in a nucleic acid sequence is by mismatch oligonucleotide mutation detection, also referred to by other names such as oligonucleotide mismatch detection. In a specific embodiment, a nucleic acid sequence comprising the site to be assayed for the mutation is amplified from a sample, such as by polymerase chain reaction, and a mutation is detected with mutation-specific oligonucleotide probe hybridization of Southern or slot blots, or a combination thereof.

In a specific embodiment of the present invention, an A908G mutation in estrogen receptor alpha nucleic acid sequence is identified by methods and/or kits employing oligonucleotide mismatch detection.

### VII. SINGLE-STRAND COMFORMATION POLYMORPHISM

Single-strand conformation polymorphism (SSCP) (Orita et al., 1989) facilitates detection of polymorphisms, such as single base pair transitions, through mobility shift analysis on a neutral polyacrylamide gel by methods well known in the art. In specific embodiments, the method is subsequent to polymerase chain reaction or restriction enzyme digestion, either of which is followed by denaturation for separation of the strands. The single stranded species are transferred onto a support such as a nylon membrane, and the mobility shift is detected by hybridization with a nick-translated DNA fragment or with RNA. In alternative embodiments, the single stranded product is itself labeled, such as with radioactivity, for identification. Samples manifesting migration shifts in SSCP gels in a specific embodiment are analyzed further by other well known methods, such as by DNA sequencing.

In a specific embodiment of the present invention, an A908G mutation in estrogen receptor alpha nucleic acid sequence is identified by methods and/or kits employing single-strand conformation polymorphism.

### VIII. THERAPEUTIC EMBODIMENTS OF THE INVENTION

In some embodiments of the invention, therapeutic methods and/or compositions are provided. In certain aspects, the therapeutic methods and/or compositions concern overcoming the resistance to therapy that results directly or indirectly from having the K303R mutation in ERα.

In one embodiment, the therapeutic composition of the invention comprises one or more peptides. In a specific embodiment, the peptide comprises the estrogen receptor alpha K303 amino acid. In particular cases, the peptide is of a particular length, for example at least 3, 4, 5, 6, 7, 8, 9, 10, 11, 12, 13, 14, 15, 16, 17, 18, 19, 20, 21, 22, 23, 24, 25, 26, 27, 28, 29, or 30 or more amino acids in length. In other particular cases, the peptide is no more than 3, 4, 5, 6, 7, 8, 9, 10, 11, 12, 13, 14, 15, 16, 17, 18, 19, 20, 21, 22, 23, 24, 25, 26, 27, 28, 29, or 30 or more amino acids in length, for example. The peptide may be from 5-10, 5-15, 5-20, 5-25, 5-30, 7-10, 7-15, 7-20, 7-25, 7-30, 10-15, 10-20, 10-25, 10-30, 12-15, 12-20, 12-25, 12-30, 15-20, 15-25, 15-30, 20-25, 20-30, or 25-30 amino acids in length, for example.

In particular embodiments of the peptide, there is a mutation at the estrogen receptor alpha K303 amino acid. In specific cases, the peptide is or is not capable of being phosphorylated at the estrogen receptor alpha K303 amino acid.

In an exemplary embodiment, the ERαL residue ²⁹⁸ IKRSK*KNSLALSC³¹⁰, (SEQ ID NO:24) where *K=is the ERαL residue where the wild-type 303 residue K is mutated to R, is provided in a peptide. In specific embodiments, this residue is modified. In further specific embodiments, it is modified such that it cannot be phosphorylated. In a specific embodiment, S305 can be phosphorylated better with the K303R mutation. In certain cases, the mutation causes a loss of acetylation. In an additional specific embodiment, the residue is acetylated (for example, after peptide synthesis), although in other embodiments the residue is modified in another way, for example the K302/K303 residue can be methylated, acetylated, or sumoylated. Peptides of the invention may be at least 70%, 75%, 80%, 85%, 90%, 92%, 93%, 95%, 97%, or 99% identical to SEQ ID NO:24, for example.

### IX. NUCLEIC ACID DETECTION

In addition to their use in directing the expression of estrogen receptor alpha wildtype or mutant proteins, polypeptides and/or peptides, the nucleic acid sequences disclosed herein have a variety of other uses. For example, they have utility as probes or primers for breast cancer therapy resistance embodiments involving nucleic acid hybridization.

A. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, site-directed mutagenesis, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

B. Amplification of Nucleic Acids

Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to estrogen receptor alpha wildtype or mutant are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in their entirety.

A reverse transcriptase PCR™ amplification procedure may be performed to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). Davey et al., European Application No. 329 822 disclose a nucleic acid amplification process involving cyclically synthesizing single-stranded RNA (“ssRNA”), ssDNA, and double-stranded DNA (dsDNA), which may be used in accordance with the present invention.

Miller et al., PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

C. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art. See Sambrook et al., 1989. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

D. Other Assays

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”), mass spectroscopy, and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNaseA mismatch cleavage assay that involves annealing single-stranded DNA or RNA test samples to an RNA probe, and subsequent treatment of the nucleic acid duplexes with RNaseA. For the detection of mismatches, the single-stranded products of the RNaseA treatment, electrophoretically separated according to size, are compared to similarly treated control duplexes. Samples containing smaller fragments (cleavage products) not seen in the control duplex are scored as positive.

Other investigators have described the use of RNaseI in mismatch assays. The use of RNaseI for mismatch detection is described in literature from Promega Biotech. Promega markets a kit containing RNaseI that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

### X. EXEMPLARY ESTROGEN RECEPTOR A NUCLEIC ACIDS

In a preferred embodiment, an estrogen receptor alpha nucleic acid sequence of the present invention contains an A908G mutation.

In specific embodiments, examples of the estrogen receptor alpha nucleic acid sequences that may include the A908G mutation include (using GenBank® sequence reference numbers, and the sequences are incorporated by reference herein): NM 000125.3 (SEQ ID NO:1); AF242866 (SEQ ID NO:2); AF123496.1 (SEQ ID NO:3); U47678.1 (SEQ ID NO:5); M12674.1 (SEQ ID NO:6); and X03635.1 (SEQ ID NO:7). In other specific embodiments, examples of the estrogen receptor alpha amino acid sequences that may include the K303R substitution include NP_{—}000116.2 (SEQ ID NO:8); AAF65451.1 (SEQ ID NO:9); AAD23565.1 (SEQ ID NO:10); AAB00115.1 (SEQ ID NO:11); AAA52399.1 (SEQ ID NO:12); and CAA27284.1 (SEQ ID NO:13).

The term “estrogen receptor alpha wildtype or mutant nucleic acid sequence” as used herein refers respectively to the estrogen receptor alpha wildtype nucleic acid sequence or to a mutant sequence, wherein the mutant sequence comprises an A908G mutation. The term “estrogen receptor alpha wildtype or mutant amino acid sequence” as used herein refers respectively to the estrogen receptor alpha wildtype amino acid sequence or to a mutant sequence, wherein the mutant sequence comprises an K303R mutation.

A. Nucleic Acids and Uses Thereof

Certain aspects of the present invention concern at least one estrogen receptor alpha wildtype and/or mutant nucleic acid. In certain aspects, the at least one estrogen receptor alpha wildtype and/or mutant nucleic acid comprises a wild-type or mutant estrogen receptor alpha wildtype and/or mutant nucleic acid. In certain aspects, the estrogen receptor alpha wildtype and/or mutant nucleic acid comprises at least one transcribed nucleic acid. In particular aspects, the estrogen receptor alpha wildtype and/or mutant nucleic acid encodes at least one estrogen receptor alpha wildtype and/or mutant protein, polypeptide or peptide, or biologically functional equivalent thereof. In other aspects, the estrogen receptor alpha wildtype and/or mutant nucleic acid comprises at least one nucleic acid segment of SEQ ID NO:1, SEQ ID NO:2, SEQ ID NO:3, SEQ ID NO:5, SEQ ID NO:6, SEQ ID NO:7, or at least one biologically functional equivalent thereof.

The present invention also concerns the isolation or creation of at least one recombinant construct or at least one recombinant host cell through the application of recombinant nucleic acid technology known to those of skill in the art or as described herein. The recombinant construct or host cell may comprise at least one estrogen receptor alpha wildtype or mutant nucleic acid, and may express at least one estrogen receptor alpha wildtype or mutant protein, peptide or peptide, or at least one biologically functional equivalent thereof.

As used herein “wild-type” refers to the naturally occurring sequence of a nucleic acid at a genetic locus in the genome of an organism, and sequences transcribed or translated from such a nucleic acid. Thus, the term “wild-type” also may refer to the amino acid sequence encoded by the nucleic acid. As a genetic locus may have more than one sequence or alleles in a population of individuals, the term “wild-type” encompasses all such naturally occurring alleles. As used herein the term “polymorphic” means that variation exists (i.e. two or more alleles exist) at a genetic locus in the individuals of a population. As used herein “mutant” refers to a change in the sequence of a nucleic acid or its encoded protein, polypeptide or peptide that is the result of the hand of man.

A nucleic acid may be made by any technique known to one of ordinary skill in the art. Non-limiting examples of synthetic nucleic acid, particularly a synthetic oligonucleotide, include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986, and U.S. Pat. No. 5,705,629, each incorporated herein by reference. A non-limiting example of enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of oligonucleotides described in U.S. Pat. No. 5,645,897, incorporated herein by reference. A non-limiting example of a biologically produced nucleic acid includes recombinant nucleic acid production in living cells, such as recombinant DNA vector production in bacteria (see for example, Sambrook et al. 1989, incorporated herein by reference).

A nucleic acid may be purified on polyacrylamide gels, agarose, cesium chloride centrifugation gradients, or by any other means known to one of ordinary skill in the art (see for example, Sambrook et al. 1989, incorporated herein by reference).

The term “nucleic acid” will generally refer to at least one molecule or strand of DNA, RNA or a derivative or mimic thereof, comprising at least one nucleobase, such as, for example, a naturally occurring purine or pyrimidine base found in DNA (e.g. adenine “A,” guanine “G,” thymine “T” and cytosine “C”) or RNA (e.g. A, G, uracil “U” and C). The term “nucleic acid” encompass the terms “oligonucleotide” and “polynucleotide.” The term “oligonucleotide” refers to at least one molecule of between about 3 and about 100 nucleobases in length. The term “polynucleotide” refers to at least one molecule of greater than about 100 nucleobases in length. These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss”, a double stranded nucleic acid by the prefix “ds”, and a triple stranded nucleic acid by the prefix “ts.”

Thus, the present invention also encompasses at least one nucleic acid that is complementary to a estrogen receptor alpha wildtype or mutant nucleic acid. In particular embodiments the invention encompasses at least one nucleic acid or nucleic acid segment complementary to the sequence set forth in SEQ ID NO:1, SEQ ID NO:2, SEQ ID NO:3, SEQ ID NO:5, SEQ ID NO:6, or SEQ ID NO:7. Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” also refers to nucleic acid(s) that are substantially complementary, as may be assessed by the same nucleotide comparison set forth above. The term “substantially complementary” refers to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double stranded nucleic acid molecule during hybridization. In certain embodiments, the term “substantially complementary” refers to at least one nucleic acid that may hybridize to at least one nucleic acid strand or duplex in stringent conditions. In certain embodiments, a “partly complementary” nucleic acid comprises at least one sequence that may hybridize in low stringency conditions to at least one single or double stranded nucleic acid, or contains at least one sequence in which less than about 70% of the nucleobase sequence is capable of base-pairing with at least one single or double stranded nucleic acid molecule during hybridization.

As used herein, “hybridization”, “hybridizes” or “capable of hybridizing” is understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating at least one nucleic acid, such as a gene or nucleic acid segment thereof, or detecting at least one specific mRNA transcript or nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence of formamide, tetramethylammonium chloride or other solvent(s) in the hybridization mixture. It is generally appreciated that conditions may be rendered more stringent, such as, for example, the addition of increasing amounts of formamide.

It is also understood that these ranges, compositions and conditions for hybridization are mentioned by way of non-limiting example only, and that the desired stringency for a particular hybridization reaction is often determined empirically by comparison to one or more positive or negative controls. Depending on the application envisioned it is preferred to employ varying conditions of hybridization to achieve varying degrees of selectivity of the nucleic acid(s) towards target sequence(s). In a non-limiting example, identification or isolation of related target nucleic acid(s) that do not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

One or more nucleic acid(s) may comprise, or be composed entirely of, at least one derivative or mimic of at least one nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refers to a molecule that may or may not structurally resemble a naturally occurring molecule, but functions similarly to the naturally occurring molecule. As used herein, a “moiety” generally refers to a smaller chemical or molecular component of a larger chemical or molecular structure, and is encompassed by the term “molecule.”

As used herein a “nucleobase” refers to a naturally occurring heterocyclic base, such as A, T, G, C or U (“naturally occurring nucleobase(s)”), found in at least one naturally occurring nucleic acid (i.e. DNA and RNA), and their naturally or non-naturally occurring derivatives and mimics. Non-limiting examples of nucleobases include purines and pyrimidines, as well as derivatives and mimics thereof, which generally can form one or more hydrogen bonds (“anneal” or “hybridize”) with at least one naturally occurring nucleobase in manner that may substitute for naturally occurring nucleobase pairing (e.g. the hydrogen bonding between A and T, G and C, and A and U).

Nucleobase, nucleoside and nucleotide mimics or derivatives are well known in the art, and have been described in exemplary references such as, for example, Scheit, Nucleotide Analogs (John Wiley, New York, 1980), incorporated herein by reference. “Purine” and “pyrimidine” nucleobases encompass naturally occurring purine and pyrimidine nucleobases and also derivatives and mimics thereof, including but not limited to, those purines and pyrimidines substituted by one or more of alkyl, carboxyalkyl, amino, hydroxyl, halogen (i.e. fluoro, chloro, bromo, or iodo), thiol, or alkylthiol wherein the alkyl group comprises of from about 1, about 2, about 3, about 4, about 5, to about 6 carbon atoms. Non-limiting examples of purines and pyrimidines include deazapurines, 2,6-diaminopurine, 5-fluorouracil, xanthine, hypoxanthine, 8-bromoguanine, 8-chloroguanine, bromothymine, 8-aminoguanine, 8-hydroxyguanine, 8-methylguanine, 8-thioguanine, azaguanines, 2-aminopurine, 5-ethylcytosine, 5-methylcyosine, 5-bromouracil, 5-ethyluracil, 5-iodouracil, 5-chlorouracil, 5-propyluracil, thiouracil, 2-methyladenine, methylthioadenine, N,N-diemethyladenine, azaadenines, 8-bromoadenine, 8-hydroxyadenine, 6-hydroxyaminopurine, 6-thiopurine, 4-(6-aminohexyl/cytosine), and the like.

As used herein, “nucleoside” refers to an individual chemical unit comprising a nucleobase covalently attached to a nucleobase linker moiety. A non-limiting example of a “nucleobase linker moiety” is a sugar comprising 5-carbon atoms (a “5-carbon sugar”), including but not limited to deoxyribose, ribose or arabinose, and derivatives or mimics of 5-carbon sugars. Non-limiting examples of derivatives or mimics of 5-carbon sugars include 2′-fluoro-2′-deoxyribose or carbocyclic sugars where a carbon is substituted for the oxygen atom in the sugar ring. By way of non-limiting example, nucleosides comprising purine (i.e. A and G) or 7-deazapurine nucleobases typically covalently attach the 9 position of the purine or 7-deazapurine to the 1′-position of a 5-carbon sugar. In another non-limiting example, nucleosides comprising pyrimidine nucleobases (i.e. C, T or U) typically covalently attach the 1 position of the pyrimidine to 1′-position of a 5-carbon sugar (Kornberg and Baker, DNA Replication, 2nd Ed. (Freeman, San Francisco, 1992). However, other types of covalent attachments of a nucleobase to a nucleobase linker moiety are known in the art, and non-limiting examples are described herein.

As used herein, a “nucleotide” refers to a nucleoside further comprising a “backbone moiety” generally used for the covalent attachment of one or more nucleotides to another molecule or to each other to form one or more nucleic acids. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when the nucleotide comprises derivatives or mimics of a naturally occurring 5-carbon sugar or phosphorus moiety, and non-limiting examples are described herein.

A non-limiting example of a nucleic acid comprising such nucleoside or nucleotide derivatives and mimics is a “polyether nucleic acid”, described in U.S. Pat. No. 5,908,845, incorporated herein by reference, wherein one or more nucleobases are linked to chiral carbon atoms in a polyether backbone. Another example of a nucleic acid comprising nucleoside or nucleotide derivatives or mimics is a “peptide nucleic acid”, also known as a “PNA”, “peptide-based nucleic acid mimics” or “PENAMs”, described in U.S. Pat. Nos. 5,786,461, 5891,625, 5,773,571, 5,766,855, 5,736,336, 5,719,262, 5,714,331, 5,539,082, and WO 92/20702, each of which is incorporated herein by reference. A peptide nucleic acid generally comprises at least one nucleobase and at least one nucleobase linker moiety that is either not a 5-carbon sugar and/or at least one backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

Peptide nucleic acids generally have enhanced sequence specificity, binding properties, and resistance to enzymatic degradation in comparison to molecules such as DNA and RNA (Egholm et al., Nature 1993, 365, 566; PCT/EP/01219). In addition, U.S. Pat. Nos. 5,766,855, 5,719,262, 5,714,331 and 5,736,336 describe PNAs comprising naturally and non-naturally occurring nucleobases and alkylamine side chains with further improvements in sequence specificity, solubility and binding affinity. These properties promote double or triple helix formation between a target nucleic acid and the PNA.

U.S. Pat. No. 5,641,625 describes that the binding of a PNA may to a target sequence has applications the creation of PNA probes to nucleotide sequences, modulating (i.e. enhancing or reducing) gene expression by binding of a PNA to an expressed nucleotide sequence, and cleavage of specific dsDNA molecules. In certain embodiments, nucleic acid analogues such as one or more peptide nucleic acids may be used to inhibit nucleic acid amplification, such as in PCR, to reduce false positives and discriminate between single base mutants, as described in U.S. Pat. No. 5,891,625.

U.S. Pat. No. 5,786,461 describes PNAs with amino acid side chains attached to the PNA backbone to enhance solubility. The neutrality of the PNA backbone may contribute to the thermal stability of PNA/DNA and PNA/RNA duplexes by reducing charge repulsion. The melting temperature of PNA containing duplexes, or temperature at which the strands of the duplex release into single stranded molecules, has been described as less dependent upon salt concentration.

One method for increasing amount of cellular uptake property of PNAs is to attach a lipophilic group. U.S. application Ser. No. 117,363, filed Sep. 3, 1993, describes several alkylamino functionalities and their use in the attachment of such pendant groups to oligonucleosides. U.S. application Ser. No. 07/943,516, filed Sep. 11, 1992, and its corresponding published PCT application WO 94/06815, describe other novel amine-containing compounds and their incorporation into oligonucleotides for, inter alia, the purposes of enhancing cellular uptake, increasing lipophilicity, causing greater cellular retention and increasing the distribution of the compound within the cell.

Additional non-limiting examples of nucleosides, nucleotides or nucleic acids comprising 5-carbon sugar and/or backbone moiety derivatives or mimics are well known in the art.

In certain aspect, the present invention concerns at least one nucleic acid that is an isolated nucleic acid. As used herein, the term “isolated nucleic acid” refers to at least one nucleic acid molecule that has been isolated free of, or is otherwise free of, the bulk of the total genomic and transcribed nucleic acids of one or more cells, particularly mammalian cells, and more particularly human cells. In certain embodiments, “isolated nucleic acid” refers to a nucleic acid that has been isolated free of, or is otherwise free of, bulk of cellular components and macromolecules such as lipids, proteins, small biological molecules, and the like. As different species may have a RNA or a DNA containing genome, the term “isolated nucleic acid” encompasses both the terms “isolated DNA” and “isolated RNA”. Thus, the isolated nucleic acid may comprise a RNA or DNA molecule isolated from, or otherwise free of, the bulk of total RNA, DNA or other nucleic acids of a particular species. As used herein, an isolated nucleic acid isolated from a particular species is referred to as a “species specific nucleic acid.” When designating a nucleic acid isolated from a particular species, such as human, such a type of nucleic acid may be identified by the name of the species. For example, a nucleic acid isolated from one or more humans would be an “isolated human nucleic acid”, a nucleic acid isolated from human would be an “isolated human nucleic acid”, and so forth.

Of course, more than one copy of an isolated nucleic acid may be isolated from biological material, or produced in vitro, using standard techniques that are known to those of skill in the art. In particular embodiments, the isolated nucleic acid is capable of expressing a protein, polypeptide or peptide that has the K303R substitution. In other embodiments, the isolated nucleic acid comprises an isolated estrogen receptor alpha wildtype or mutant nucleic acid sequence.

Herein certain embodiments, a “gene” refers to a nucleic acid that is transcribed. As used herein, a “gene segment” is a nucleic acid segment of a gene. In certain aspects, the gene includes regulatory sequences involved in transcription, or message production or composition. In particular embodiments, the gene comprises transcribed sequences that encode for a protein, polypeptide or peptide. In other particular aspects, the gene comprises an estrogen receptor alpha wildtype or mutant nucleic acid, and/or encodes an estrogen receptor alpha wildtype or mutant polypeptide or peptide coding sequences. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof. In particular aspects, the transcribed nucleotide sequence comprises at least one functional protein, polypeptide and/or peptide encoding unit. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

“Isolated substantially away from other coding sequences” means that the gene of interest, in this case the estrogen receptor alpha gene(s) containing the A908G mutation, forms the significant part of the coding region of the nucleic acid, or that the nucleic acid does not contain large portions of naturally-occurring coding nucleic acids, such as large chromosomal fragments, other functional genes, RNA or cDNA coding regions. Of course, this refers to the nucleic acid as originally isolated, and does not exclude genes or coding regions later added to the nucleic acid by the hand of man.

In certain embodiments, the nucleic acid is a nucleic acid segment. As used herein, the term “nucleic acid segment”, are smaller fragments of a nucleic acid, such as for non-limiting example, those that encode only part of the estrogen receptor alpha wildtype or mutant peptide or polypeptide sequence. In a preferred embodiment, the mutant peptide or polypeptide sequence comprises the K303R substitution. Thus, a “nucleic acid segment” may comprise any part of the estrogen receptor alpha wildtype or mutant gene sequence(s), of from about 2 nucleotides to the full length of the estrogen receptor alpha wildtype or mutant peptide or polypeptide encoding region. In certain embodiments, the “nucleic acid segment” encompasses the full length estrogen receptor alpha wildtype or mutant gene(s) sequence. In particular embodiments, the nucleic acid comprises any part of the SEQ ID NO:1, SEQ ID NO:2, SEQ ID NO:3, SEQ ID NO:5, SEQ ID NO:6, or SEQ ID NO:7 sequence(s), of from about 2 nucleotides to the full length of the sequence disclosed in SEQ ID NO:1, SEQ ID NO:2, SEQ ID NO:3, SEQ ID NO:5, SEQ ID NO:6, or SEQ ID NO:7.

A non-limiting example of the present invention would be the generation of nucleic acid segments of various lengths and sequence composition for probes and primers based on the sequences disclosed in SEQ ID NO:1, SEQ ID NO:2, SEQ ID NO:3, SEQ ID NO:5, SEQ ID NO:6, or SEQ ID NO:7.

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). The length overall length may vary considerably between nucleic acid constructs. Thus, a nucleic acid segment of almost any length may be employed, with the total length preferably being limited by the ease of preparation or use in the intended recombinant nucleic acid protocol.

In a non-limiting example, one or more nucleic acid constructs may be prepared that include a contiguous stretch of nucleotides identical to or complementary to SEQ ID NO:1, SEQ ID NO:2, SEQ ID NO:3, SEQ ID NO:5, SEQ ID NO:6, or SEQ ID NO:7. A nucleic acid construct may be about 3, about 5, about 8, about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e. all integers including and between such values). Non-limiting examples of intermediate lengths include about 11, about 12, about 13, about 16, about 17, about 18, about 19, etc.; about 21, about 22, about 23, etc.; about 31, about 32, etc.; about 51, about 52, about 53, etc.; about 101, about 102, about 103, etc.; about 151, about 152, about 153, etc.; about 1,001, about 1002, etc.; about 50,001, about 50,002, etc; about 750,001, about 750,002, etc.; about 1,000,001, about 1,000,002, etc. Non-limiting examples of intermediate ranges include about 3 to about 32, about 150 to about 500,001, about 3,032 to about 7,145, about 5,000 to about 15,000, about 20,007 to about 1,000,003, etc.

In particular embodiments, the invention concerns one or more recombinant vector(s) comprising nucleic acid sequences that encode an estrogen receptor alpha wildtype or mutant protein, polypeptide or peptide that includes within its amino acid sequence a contiguous amino acid sequence in accordance with, or essentially as set forth in SEQ ID NO:8, SEQ ID NO:9, SEQ ID NO:10, SEQ ID NO:11, SEQ ID NO:12, or SEQ ID NO:13. In other embodiments, the invention concerns recombinant vector(s) comprising nucleic acid sequences that encode a human estrogen receptor alpha wildtype or mutant protein, polypeptide or peptide that includes within its amino acid sequence a contiguous amino acid sequence in accordance with, or essentially as set forth in SEQ ID NO:8, SEQ ID NO:9, SEQ ID NO:10, SEQ ID NO:11, SEQ ID NO:12, or SEQ ID NO:13. In particular aspects, the recombinant vectors are DNA vectors.

The term “a sequence essentially as set forth in SEQ ID NO:8” means that the sequence substantially corresponds to a portion of SEQ ID NO:8 and has relatively few amino acids that are not identical to, or a biologically functional equivalent of, the amino acids of SEQ ID NO:8. Thus, “a sequence essentially as set forth in SEQ ID NO:1 encompasses nucleic acids, nucleic acid segments, and genes that comprise part or all of the nucleic acid sequences as set forth in SEQ ID NO:1.

The term “biologically functional equivalent” is well understood in the art and is further defined in detail herein. Accordingly, a sequence that has between about 70% and about 80%; or more preferably, between about 81% and about 90%; or even more preferably, between about 91% and about 99%; of amino acids that are identical or functionally equivalent to the amino acids of SEQ ID NO:8, SEQ ID NO:9, SEQ ID NO:10, SEQ ID NO:11, SEQ ID NO:12, or SEQ ID NO:13 will be a sequence that is “essentially as set forth in SEQ ID NO:8, SEQ ID NO:9, SEQ ID NO:10, SEQ ID NO:11, SEQ ID NO:12, or SEQ ID NO:13”, provided the biological activity of the protein, polypeptide or peptide is maintained.

In certain other embodiments, the invention concerns at least one recombinant vector that include within its sequence a nucleic acid sequence essentially as set forth in SEQ ID NO:1, SEQ ID NO:2, SEQ ID NO:3, SEQ ID NO:5, SEQ ID NO:6, or SEQ ID NO:7. In particular embodiments, the recombinant vector comprises DNA sequences that encode protein(s), polypeptide(s) or peptide(s) exhibiting estrogen receptor alpha wildtype or mutant activity.

The term “functionally equivalent codon” is used herein to refer to codons that encode the same amino acid, such as the six codons for arginine and serine, and also refers to codons that encode biologically equivalent amino acids, which are well known in the art.

Information on codon usage in a variety of non-human organisms is known in the art (see for example, Bennetzen and Hall, 1982; Ikemura, 1981a, 1981b, 1982; Grantham et al., 1980, 1981; Wada et al., 1990; each of these references are incorporated herein by reference in their entirety). Thus, it is contemplated that codon usage may be optimized for other animals, as well as other organisms such as fungi, plants, prokaryotes, virus and the like, as well as organelles that contain nucleic acids, such as mitochondria, chloroplasts and the like, based on the preferred codon usage as would be known to those of ordinary skill in the art.

It will also be understood that amino acid sequences or nucleic acid sequences may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

Excepting intronic and flanking regions, and allowing for the degeneracy of the genetic code, nucleic acid sequences that have between about 70% and about 79%; or more preferably, between about 80% and about 89%; or even more particularly, between about 90% and about 99%; of nucleotides that are identical to the nucleotides of SEQ ID NO:1, SEQ ID NO:2, SEQ ID NO:3, SEQ ID NO:5, SEQ ID NO:6, or SEQ ID NO:7 will be nucleic acid sequences that are “essentially as set forth in SEQ ID NO:1, SEQ ID NO:2, SEQ ID NO:3, SEQ ID NO:5, SEQ ID NO:6, or SEQ ID NO:7”.

It will also be understood that this invention is not limited to the particular nucleic acid sequences of SEQ ID NO:1, SEQ ID NO:2, SEQ ID NO:3, SEQ ID NO:5, SEQ ID NO:6, or SEQ ID NO:7, or the amino acid sequences of SEQ ID NO:8, SEQ ID NO:9, SEQ ID NO:10, SEQ ID NO:11, SEQ ID NO:12, or SEQ ID NO:13, respectively. Recombinant vectors and isolated nucleic acid segments may therefore variously include these coding regions themselves, coding regions bearing selected alterations or modifications in the basic coding region, and they may encode larger polypeptides or peptides that nevertheless include such coding regions or may encode biologically functional equivalent proteins, polypeptide or peptides that have mutant amino acids sequences.

The nucleic acids of the present invention encompass biologically functional equivalent estrogen receptor alpha wildtype or mutant proteins, polypeptides, or peptides. Such sequences may arise as a consequence of codon redundancy or functional equivalency that are known to occur naturally within nucleic acid sequences or the proteins, polypeptides or peptides thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Changes designed by man may be introduced, for example, through the application of site-directed mutagenesis techniques as discussed herein below, e.g., to introduce improvements or alterations to the antigenicity of the protein, polypeptide or peptide, or to test mutants in order to examine estrogen receptor alpha wildtype or mutant protein, polypeptide or peptide activity at the molecular level.

Fusion proteins, polypeptides or peptides may be prepared, e.g., where the estrogen receptor alpha wildtype or mutant coding regions are aligned within the same expression unit with other proteins, polypeptides or peptides having desired functions. Non-limiting examples of such desired functions of expression sequences include purification or immunodetection purposes for the added expression sequences, e.g., proteinaceous compositions that may be purified by affinity chromatography or the enzyme labeling of coding regions, respectively.

Encompassed by the invention are nucleic acid sequences encoding relatively small peptides or fusion peptides, such as, for example, peptides of from about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, to about 100 amino acids in length, or more preferably, of from about 15 to about 30 amino acids in length; as set forth in SEQ ID NO:8, SEQ ID NO:9, SEQ ID NO:10, SEQ ID NO:11, SEQ ID NO:12, or SEQ ID NO:13, and also larger polypeptides up to and including proteins corresponding to the full-length sequences set forth in SEQ ID NO:8, SEQ ID NO:9, SEQ ID NO:10, SEQ ID NO:11, SEQ ID NO:12, or SEQ ID NO:13.

As used herein an “organism” may be a prokaryote, eukaryote, virus and the like. As used herein the term “sequence” encompasses both the terms “nucleic acid” and “proteinaceous” or “proteinaceous composition.” As used herein, the term “proteinaceous composition” encompasses the terms “protein”, “polypeptide” and “peptide.” As used herein “artificial sequence” refers to a sequence of a nucleic acid not derived from sequence naturally occurring at a genetic locus, as well as the sequence of any proteins, polypeptides or peptides encoded by such a nucleic acid. A “synthetic sequence”, refers to a nucleic acid or proteinaceous composition produced by chemical synthesis in vitro, rather than enzymatic production in vitro (i.e. an “enzymatically produced” sequence) or biological production in vivo (i.e. a “biologically produced” sequence).

### XI. IMMUNODETECTION METHODS

In still further embodiments, the present invention concerns immunodetection methods for binding, purifying, removing, quantifying and/or otherwise generally detecting biological components such as estrogen receptor alpha protein or nucleic acid components. The estrogen receptor alpha antibodies prepared in accordance with the present invention may be employed to detect wild-type and/or mutant estrogen receptor alpha proteins, polypeptides and/or peptides. In specific embodiments, the antibodies detect an acetylated form of estrogen receptor alpha protein, polypeptide and/or peptide or the antibodies detect an K303R estrogen receptor alpha amino acid mutation. The use of wild-type and/or mutant estrogen receptor alpha specific antibodies is contemplated. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle M H and Ben-Zeev O, 1999; Gulbis B and Galand P, 1993; De Jager R et al., 1993; and Nakamura et al., 1987, each incorporated herein by reference.

In general, the immunobinding methods include obtaining a sample suspected of containing estrogen receptor alpha protein, polypeptide and/or peptide, and contacting the sample with a first anti-estrogen receptor alpha antibody in accordance with the present invention, as the case may be, under conditions effective to allow the formation of immunocomplexes.

These methods include methods for purifying wild-type and/or mutant estrogen receptor alpha proteins, polypeptides and/or peptides as may be employed in purifying wild-type and/or mutant estrogen receptor alpha proteins, polypeptides and/or peptides from patients' samples and/or for purifying recombinantly expressed wild-type or mutant estrogen receptor alpha proteins, polypeptides and/or peptides. In these instances, the antibody removes the antigenic wild-type and/or mutant estrogen receptor alpha protein, polypeptide and/or peptide component from a sample. The antibody will preferably be linked to a solid support, such as in the form of a column matrix, and the sample suspected of containing the wild-type or mutant estrogen receptor alpha protein antigenic component will be applied to the immobilized antibody. The unwanted components will be washed from the column, leaving the antigen immunocomplexed to the immobilized antibody, which wild-type or mutant estrogen receptor alpha protein antigen is then collected by removing the wild-type or mutant estrogen receptor alpha protein and/or peptide from the column.

The immunobinding methods also include methods for detecting and quantifying the amount of a wild-type or mutant estrogen receptor alpha protein reactive component in a sample and the detection and quantification of any immune complexes formed during the binding process. Here, one would obtain a sample suspected of containing a wild-type or mutant estrogen receptor alpha protein and/or peptide, and contact the sample with an antibody against wild-type or mutant estrogen receptor alpha, and then detect and quantify the amount of immune complexes formed under the specific conditions.

In terms of antigen detection, the biological sample analyzed may be any sample that is suspected of containing a wild-type or mutant estrogen receptor alpha protein-specific antigen, such as a breast tissue section or specimen, a homogenized breast tissue extract, a breast cell, separated and/or purified forms of any of the above wild-type or mutant estrogen receptor alpha protein-containing compositions, or even any biological fluid that comes into contact with the breast tissue. Diseases that may be suspected of containing a wild-type or mutant estrogen receptor alpha protein-specific antigen include, but are not limited to, breast cancer.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any estrogen receptor alpha protein antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Pat. Nos. concerning the use of such labels include 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The estrogen receptor alpha antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

The immunodetection methods of the present invention have evident utility in the diagnosis and prognosis of conditions such as various forms of cancer, such as breast cancer. Here, a biological and/or clinical sample suspected of containing a wild-type or mutant estrogen receptor alpha protein, polypeptide, peptide and/or mutant is used. However, these embodiments also have applications to non-clinical samples, such as in the titering of antigen or antibody samples, for example in the selection of hybridomas.

In the clinical diagnosis and/or monitoring of patients with various forms of breast cancer, the detection of estrogen receptor alpha mutant, and/or an alteration in the levels of estrogen receptor alpha, in comparison to the levels in a corresponding biological sample from a normal subject is indicative of a patient with cancer, such as breast cancer. However, as is known to those of skill in the art, such a clinical diagnosis would not necessarily be made on the basis of this method in isolation. Those of skill in the art are very familiar with differentiating between significant differences in types and/or amounts of biomarkers, which represent a positive identification, and/or low level and/or background changes of biomarkers. Indeed, background expression levels are often used to form a “cut-off” above which increased detection will be scored as significant and/or positive.

A. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, the anti-estrogen receptor alpha antibodies of the invention are immobilized onto a selected surface exhibiting protein affinity, such as a well in a polystyrene microtiter plate. Then, a test composition suspected of containing the wild-type and/or mutant estrogen receptor alpha protein antigen, such as a clinical sample, is added to the wells. After binding and/or washing to remove non-specifically bound immune complexes, the bound wild-type and/or mutant estrogen receptor alpha protein antigen may be detected. Detection is generally achieved by the addition of another anti-estrogen receptor alpha antibody that is linked to a detectable label. This type of ELISA is a simple “sandwich ELISA”. Detection may also be achieved by the addition of a second anti-estrogen receptor alpha antibody, followed by the addition of a third antibody that has binding affinity for the second antibody, with the third antibody being linked to a detectable label.

In another exemplary ELISA, the samples suspected of containing the wild-type and/or mutant estrogen receptor alpha protein antigen are immobilized onto the well surface and/or then contacted with the anti-estrogen receptor alpha antibodies of the invention. After binding and/or washing to remove non-specifically bound immune complexes, the bound anti-estrogen receptor alpha antibodies are detected. Where the initial anti-estrogen receptor alpha antibodies are linked to a detectable label, the immune complexes may be detected directly. Again, the immune complexes may be detected using a second antibody that has binding affinity for the first anti-estrogen receptor alpha antibody, with the second antibody being linked to a detectable label.

Another ELISA in which the wild-type and/or mutant estrogen receptor alpha proteins, polypeptides and/or peptides are immobilized, involves the use of antibody competition in the detection. In this ELISA, labeled antibodies against wild-type or mutant estrogen receptor alpha protein are added to the wells, allowed to bind, and/or detected by means of their label. The amount of wild-type or mutant estrogen receptor alpha protein antigen in an unknown sample is then determined by mixing the sample with the labeled antibodies against wild-type and/or mutant estrogen receptor alpha before and/or during incubation with coated wells. The presence of wild-type and/or mutant estrogen receptor alpha protein in the sample acts to reduce the amount of antibody against wild-type or mutant estrogen receptor alpha protein available for binding to the well and thus reduces the ultimate signal. This is also appropriate for detecting antibodies against wild-type or mutant estrogen receptor alpha protein in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

B. Immunohistochemistry

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

C. Immunodetection Kits

In still further embodiments, the present invention concerns immunodetection kits for use with the immunodetection methods described above. As the estrogen receptor alpha antibodies are generally used to detect wild-type and/or mutant estrogen receptor alpha proteins, polypeptides and/or peptides, or to detect the A908G mutation in estrogen receptor nucleic acid sequence, the antibodies will preferably be included in the kit. However, kits including both such components may be provided. The immunodetection kits will thus comprise, in suitable container means, a first antibody that binds to a wild-type and/or mutant estrogen receptor alpha protein, polypeptide and/or peptide, and/or optionally, an immunodetection reagent and/or further optionally, a wild-type and/or mutant estrogen receptor alpha protein, polypeptide and/or peptide.

In preferred embodiments, monoclonal antibodies will be used. In certain embodiments, the first antibody that binds to the wild-type and/or mutant estrogen receptor alpha protein, polypeptide and/or peptide may be pre-bound to a solid support, such as a column matrix and/or well of a microtitre plate.

The immunodetection reagents of the kit may take any one of a variety of forms, including those detectable labels that are associated with and/or linked to the given antibody. Detectable labels that are associated with and/or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and/or all such labels may be employed in connection with the present invention.

The kits may further comprise a suitably aliquoted composition of the wild-type and/or mutant estrogen receptor alpha protein, polypeptide and/or polypeptide, whether labeled and/or unlabeled, as may be used to prepare a standard curve for a detection assay. The kits may contain antibody-label conjugates either in fully conjugated form, in the form of intermediates, and/or as separate moieties to be conjugated by the user of the kit. The components of the kits may be packaged either in aqueous media and/or in lyophilized form.

The container means of the kits will generally include at least one vial, test tube, flask, bottle, syringe and/or other container means, into which the antibody may be placed, and/or preferably, suitably aliquoted. Where wild-type and/or mutant estrogen receptor alpha protein, polypeptide and/or peptide, and/or a second and/or third binding ligand and/or additional component is provided, the kit will also generally contain a second, third and/or other additional container into which this ligand and/or component may be placed. The kits of the present invention will also typically include a means for containing the antibody, antigen, and/or any other reagent containers in close confinement for commercial sale. Such containers may include injection and/or blow-molded plastic containers into which the desired vials are retained.

One may be able to employ mass spectroscopy reagents such as the exemplary technology obtainable from the world wide web site of Sequenom®, for example using their iPLEX® technology.

### XII. BREAST CANCER

Tumors are notoriously heterogeneous, particularly in advanced stages of tumor progression (Morton et al., 1993; Fidler and Hart, 1982; Nowell, 1982; Elder et al., 1989; Bystryn et al., 1985). Although tumor cells within a primary tumor or metastasis all may express the same marker gene, the level of specific mRNA expression can vary considerably (Elder et al., 1989). It is, in certain instances, necessary to employ a detection system that can cope with an array of heterogeneous markers. In a specific embodiment, a marker for breast cancer comprises an A908G estrogen receptor alpha nucleic acid sequence or the K303R substitution to which it corresponds, or both.

While the present invention exemplifies various tumor suppressors as a markers, any marker that is correlated with the presence or absence of cancer may be used in combination with these markers to improve the efficacy of tumor detection and treatment. A marker, as used herein, is any proteinaceous molecule (or corresponding gene) whose production or lack of production is characteristic of a cancer cell. Depending on the particular set of markers employed in a given analysis, the statistical analysis will vary. For example, where a particular combination of markers is highly specific for melanomas or breast cancer, the statistical significance of a positive result will be high. It may be, however, that such specificity is achieved at the cost of sensitivity, i.e., a negative result may occur even in the presence of melanoma or breast cancer. By the same token, a different combination may be very sensitive, i.e., few false negatives, but has a lower specificity.

As new markers are identified, different combinations may be developed that show optimal function with different ethnic groups or sex, different geographic distributions, different stages of disease, different degrees of specificity or different degrees of sensitivity. Marker combinations also may be developed, which are particularly sensitive to the effect of therapeutic regimens on disease progression. Patients may be monitored after surgery, gene therapy, hyperthermia, immunotherapy, cytokine therapy, gene therapy, radiotherapy or chemotherapy, to determine if a specific therapy is effective.

There are many other markers that may be used in combination with these, and other, markers. For example, β-human chorionic gonadotropin (β-HCG) is produced by trophoblastic cells of placenta of pregnant woman and is essential for maintenance of pregnancy at the early stages (Pierce et al., 1981; Talmadge et al., 1984). b-HCG is known to be produced by trophoblastic or germ cell origin tumors, such as choriocarcinoma or testicular carcinoma cells (Madersbacher et al., 1994; Cole et al., 1983). Also ectopic expression of b-HCG has been detected by a number of different immunoassays in various tumors of non-gonadal such as breast, lung, gastric, colon, and pancreas, etc. (McManus et al., 1976; Yoshimura et al., 1994; Yamaguchi et al., 1989; Marcillac et al., 1992; Alfthan et al., 1992). Although the function of b-HCG production in these tumors is still unknown, the atavistic expression of b-HCG by cancer cells and not by normal cells of non-gonadal origin suggests it may be a potentially good marker in the detection of melanoma and breast cancer (Hoon et al., 1996; Sarantou et al., 1997).

Another exemplary example of a marker is glycosyltransferase b-1,4-N-acetylgalacto-saminyltransferase (GalNAc). GalNAc catalyzes the transfer of N-acetylgalactosamine by b1(r) 4 linkage onto both gangliosides GM3 and GD3 to generate GM2 and GD2, respectively (Nagata et al., 1992; Furukawa et al., 1993). It also catalyzes the transfer of N-acetylgalactosamine to other carbohydrate molecules such as mucins. Gangliosides are glycosphingolipids containing sialic acids which play an important role in cell differentiation, adhesion and malignant transformation. In melanoma, gangliosides GM2 and GD2 expression, are often enhanced to very high levels and associate with tumor progression including metastatic tumors (Hoon et al., 1989; Ando et al., 1987; Carubia et al., 1984; Tsuchida et al., 1987a), although gangliosides are also expressed in melanoma, renal, lung, breast carcinoma cancer cells. The gangliosides GM2 and GD2 are immunogenic in humans and can be used as a target for specific immunotherapy such as human monoclonal antibodies or cancer vaccines (Tsuchida et al., 1987b; Irie, 1985.)

Other markers contemplated by the present invention include cytolytic T lymphocyte (CTL) targets. MAGE-3 is a marker identified in melanoma cells and breast carcinoma. MAGE-3 is expressed in many melanomas as well as other tumors and is a (CTL) target (Gaugler et al., 1994). MAGE-1, MAGE-2, MAGE-4, MAGE-6, MAGE-12, MAGE-Xp, and are other members of the MAGE gene family. MAGE-1 gene sequence shows 73% identity with MAGE-3 and expresses an antigen also recognized by CTL (Gaugler et al., 1994). MART-1 is another potential CTL target (Robbins et al., 1994) and also may be included in the present invention.

Preferred embodiments of the invention involve many different combinations of markers for the detection of cancer cells. Any marker that is indicative of neoplasia in cells may be included in this invention. A preferred marker is an A908G estrogen receptor alpha nucleic acid sequence and/or a K303R substitution in an estrogen receptor alpha nucleic acid sequence.

### XIII. BREAST CANCER THERAPIES

There are a variety of breast cancer therapies to which an individual may become resistant. In specific embodiments, the breast cancer therapy comprises chemotherapy, hormonal therapy, and/or gene targeted therapy, e.g. using the peptide to reverse resistance. In some cases, the individual may also have had or is presently being treated with surgery or radiation, for example.

In specific embodiments, the breast cancer chemotherapy includes anthracyclines, such as doxorubicin (adriamycin), epirubicin (Ellence®), and liposomal doxorubicin (doxil); taxanes, such as docetaxel (Taxotere®), paclitaxel (Taxol®), and protein-bound paclitaxel (Abraxane®); cyclophosphamide (Cytoxan®); capecitabine (Xeloda™) and 5 fluorouracil (5 FU); vinorelbine (Navelbine®); gemcitabine (Gemzar®), trastuzumab (Herceptin®), methotrexate, mitomycin, mitozantrone (mitoxantrone), or a mixture or combination thereof. In certain cases of the invention, once a breast cancer becomes resistant to a hormone therapy, a breast cancer chemotherapy and/or a peptide of the invention is then administered to the individual. In a specific embodiment, a targeted therapy to PI3K, AKT, or IGF1R is employed.

In specific cases, the breast cancer hormonal therapy comprises one or more selective estrogen receptor modulators (SERMs). In particular embodiments, the breast cancer hormonal therapy includes Tamoxifen; Fareston®; Arimidex®; Aromasin®; Femara®; Zoladex®, or a mixture or combination thereof, for example. In another specific embodiment, selective estrogen receptor degradators are employed, such as fulvestrant, for example.

In other embodiments, the breast cancer therapy comprises aromatase inhibitor therapy. Aromatase inhibitors include compounds that inhibit the action of the enzyme aromatase, which converts androgens into estrogens by aromatization. Aromatase inhibition by a particular compound can be determined by using methods known in the art including measuring the release of tritium-labeled water upon the conversion of tritium-labeled androstenedione to estrone in the manner provided for in U.S. Pat. No. 6,803,206, which is incorporated herein by reference in its entirety.

Exemplary aromatase inhibitors useful in the invention can include, but are not limited to, anastrozole (Arimidex®), letrozole (Femara®), exemestane (Aromasin®), formestane (Lentaron), and testolactone (Teslac™). Other compounds that have shown promise as aromatase inhibitors that may be used in the present invention include, but are not limited to atamestane, vorozole (Rivizor), fadrozole (16949A), roglethimide, pyridoglutethimide, trilostane, aminoglutethimide (Cytadren™), 4-Hydroxyandrostenedione (4-OHA; Formastane), finrozole, and YM-511 (4-[N-(4-bromobenzyl)-N-(4-cyanophenyl)amino]-4H-1,2,4-triazole).

Resistance to the breast cancer therapy may be identified by detecting the mutation in ERα, or susceptibility to resistance to the breast cancer therapy may be identified. The resistance may be de novo or acquired.

### XIV. PHARMACEUTICAL PREPARATIONS

Pharmaceutical compositions of the present invention comprise an effective amount of one or more therapeutic compositions of the invention, for example, a peptide that comprises the mutation site from ERα. The composition may be dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one composition or additional active ingredient will be known to those of skill in the art in light of the present disclosure, as exemplified by Remington' s Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

In some embodiments, an effective amount of a composition of the present invention, such as an antagonist to an estrogen receptor alpha K303R polypeptide, is administered to a cell. In other embodiments, a therapeutically effective amount of a composition of the present invention is administered to an individual for the treatment of disease. The term “effective amount” as used herein is defined as the amount of a composition of the present invention which is necessary to result in a physiological change in the cell or tissue to which it is administered. The term “therapeutically effective amount” as used herein is defined as the amount of a composition of the present invention that eliminates, decreases, delays, or minimizes adverse effects of a disease, such as cancer. A skilled artisan readily recognizes that in many cases the composition may not provide a cure but may only provide partial benefit. In some embodiments, a physiological change having some benefit is also considered therapeutically beneficial. Thus, in some embodiments, an amount of a composition that provides a physiological change is considered an “effective amount” or a “therapeutically effective amount.”

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The composition may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The composition may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments, the chimeric polypeptide is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

### XV. KITS OF THE INVENTION

All the essential materials and/or reagents required for detecting estrogen receptor alpha wildtype or mutant sequences in a sample may be assembled together in a kit. This generally will comprise a probe or primers designed to be suitable for primer extension for detection of the mutation. In an alternative embodiment, the probe or primers are designed to hybridize specifically to individual nucleic acids of interest in the practice of the present invention, including estrogen receptor alpha wildtype or mutant sequences. Also included may be enzymes suitable for amplifying nucleic acids, including various polymerases (reverse transcriptase, Taq, etc.), deoxynucleotides and buffers to provide the necessary reaction mixture for amplification. Such kits may also include enzymes and other reagents suitable for detection of specific nucleic acids or amplification products. Such kits generally will comprise, in suitable means, distinct containers for each individual reagent or enzyme as well as for each probe or primer pair.

### EXAMPLES

The following examples are offered by way of example, and are not intended to limit the scope of the invention in any manner.

### Example 1

### The K303R ERα Mutation Confers Resistance to an Aromatase Inhibitor

Aromatase inhibitors are rapidly becoming the first choice for the endocrine treatment of steroid receptor positive breast cancer in postmenopausal women. An understanding of the resistance mechanisms to these agents is, therefore, essential for the appropriate treatment to responsive patients and the rational design of new diagnostic and therapeutic tools targeted at the resistance pathways.

Using MCF-7 breast cancer cells stably transfected with either wild-type (WT) or the lysine to arginine (K303R) somatic mutant ERα, which we have previously identified in premalignant and invasive breast cancers, we found that the mutant conferred a hypersensitivity to estrogen with an ability to be maximally stimulated in response to very low physiological levels of hormone. In certain embodiments of the invention, such a mutant could provide a continuous mitogenic stimulus to the breast even during phases of low circulating hormone, such as in postmenopausal women, thus affording a proliferative advantage especially during treatment with aromatase inhibitors (AIs). Phosphorylation of serine (S) 305 in ERα, which is adjacent to the site of the K303R mutation, can be mediated by several protein kinase signaling networks. The K303R ERα mutation also renders the receptor a more efficient substrate for phosphorylation at S305, which results in enhanced hormone sensitivity for growth. To develop a preclinical models to directly test whether the mutation is resistant to AIs, MCF-7 parental and MCF-7-K303R-overexpressing cell lines stably transfected with an aromatase expression vector were generated; aromatase activity levels were 27±3 μmol/h/mg protein and 43±12 μmol/h/mg protein, respectively in the transfectants. Cells were stimulated with the aromatase substrate, androstenedione (AD), with or without the AI anastrazole (AN). AN decreased AD-stimulated growth of WT vector control cells by 40%, but had no effect on AD-stimulated growth of MCF-7-K303R-overexpressing cells using in vitro MTT assays. Furthermore the basal non-stimulated colony number of the mutant cells was increased 10-fold in soft agar assays, and AN was unable to inhibit the formation of mutant colonies. Aromatase activity of both cells was reduced by 95% with AN treatment, suggesting that resistance cannot be explained by an intrinsic insensitivity of the aromatase enzyme itself to AN.

To examine the molecular mechanisms associated with AI resistance of mutant-expressing cells, short-term nongenomic signaling were examined, and the mutation generated a new site for AKT phosphorylation at S305. In addition, there were constitutively increased levels of phospho-AKT in mutant cells. To examine for effects on proliferation, immunoblot analyses were performed. K303R mutant-overexpressing cells exhibited increased levels of cyclin D1, and decreased levels of the CDK inhibitor p21 during AI treatment. Accordingly, apoptosis was altered with decreased Bax levels and PARP cleavage, increased levels of phospho-BAD, and slightly increased levels of bcl-2. These cumulative results indicate that the mutation indeed confers resistance to an AI, and potential mechanisms of resistance include cellular strategies to evade apoptosis and enhance proliferation, in specific embodiments through enhanced reception of downstream signaling by growth factor networks, such as AKT. The results indicate that the mutation is a new predictive marker of response to AIs in mutation-positive breast tumors.

### Example 2

### K303R ERα Mutation Generates an Estrogen Hypersensitive Phenotype In Vitro and In Vivo

FIG. 1 shows that the K303R ERα mutation generates an estrogen hypersensitive phenotype in vitro and in vivo. In FIG. 1A, there is immunoblotting (IB) of parental and YFP K303R ERα MCF-7 cells showing expression of endogenous ERα (66 kDa) and the exogenously added YFP-K303R-ERα (—96 kDa). Rho GDIα was used as loading control. In FIG. 1B, cells were grown in soft agar with increasing amounts of E₂ (10⁻¹² to 10⁻⁹) for 14 days. The basal colony number of the mutant cells was 2.9-fold higher compared to parental cells (p=0.0006). Both cell lines exhibited typical estradiol-induced dose-response growth curves. However, E₂ at the lowest concentration (10⁻¹²) can significantly enhance growth just in K303R expressing cells (p=0.005). In FIG. 1C, cells were injected into the mammary fat pad of athymic nude mice supplemented with an estrogen pellet (80 pg/ml). K303R-ERα tumors grew more quickly than the WT-ERα tumors (p=0.0466, chi-square). Results shown are time for tumor size to triple.

### Example 3

### The K303R ERα Mutation Confers Resistance to the Aromatase Inhibitor Anastrazole (Ana)

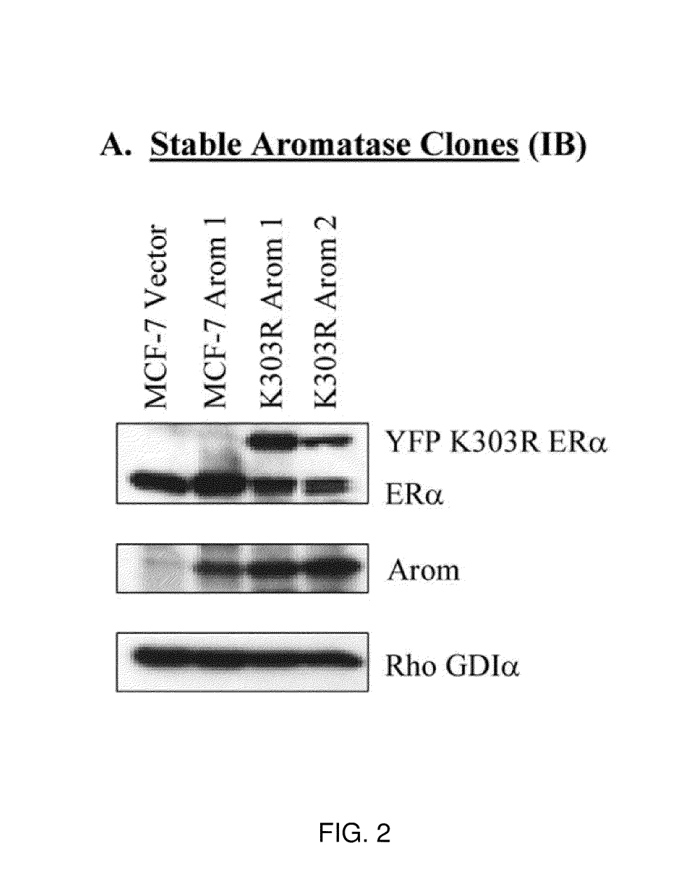

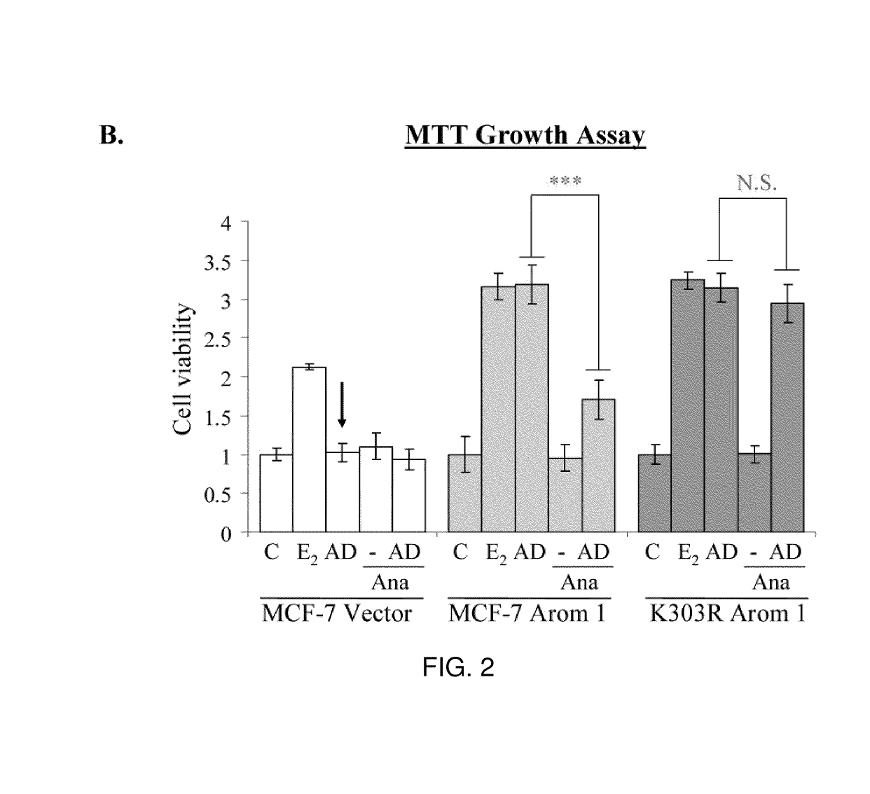

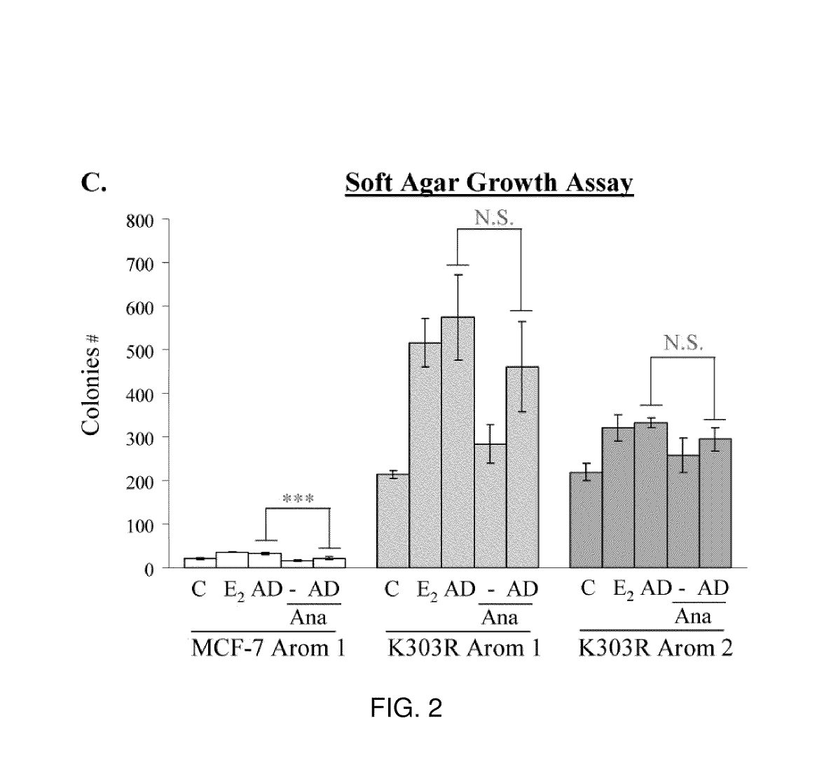

FIG. 2 shows that the K303R ERα mutation confers resistance to the exemplary aromatase inhibitor Anastrazole (Ana). In FIG. 2A, MCF-7 parental and K303R-expressing cells were stably transfected with an aromatase cDNA expression vector. IB analysis showing aromatase overexpression. In FIG. 2B, there is a MTT Growth Assay in MCF-7 vector, MCF-7 Arom 1 and K303R Arom 1 cells treated for 9 days with vehicle (C), estrogen (E₂, 1 nM), androstenedione (AD, 10 nM) and/or anastrazole (Ana 1 μM). Cell proliferation is expressed as fold compared with C. Ana decreased AD-stimulated growth by 47% in MCF-7 Arom 1 cells (p=0.0002), but had no effect on AD-stimulated growth of MCF-7 K303R Arom 1 cells. C. Soft agar growth assay in MCF-7 Arom 1 and K303R Arom 1 and 2 cells. Cells were stimulated for 14 days with the above mentioned treatments. The basal nonstimulated colony number was increased by 10-fold in K303R Arom 1 and 2 compared to MCF-7 Arom 1 (p<0.0001), and the AI Ana did not inhibit the AD-induced formation of colonies. Therefore, the mutation can generate an AI^{R} phenotype.

### Example 4

### The Pi-3K/Akt Pathway is Involved in K303R-Associated AI^{R} Phenotype

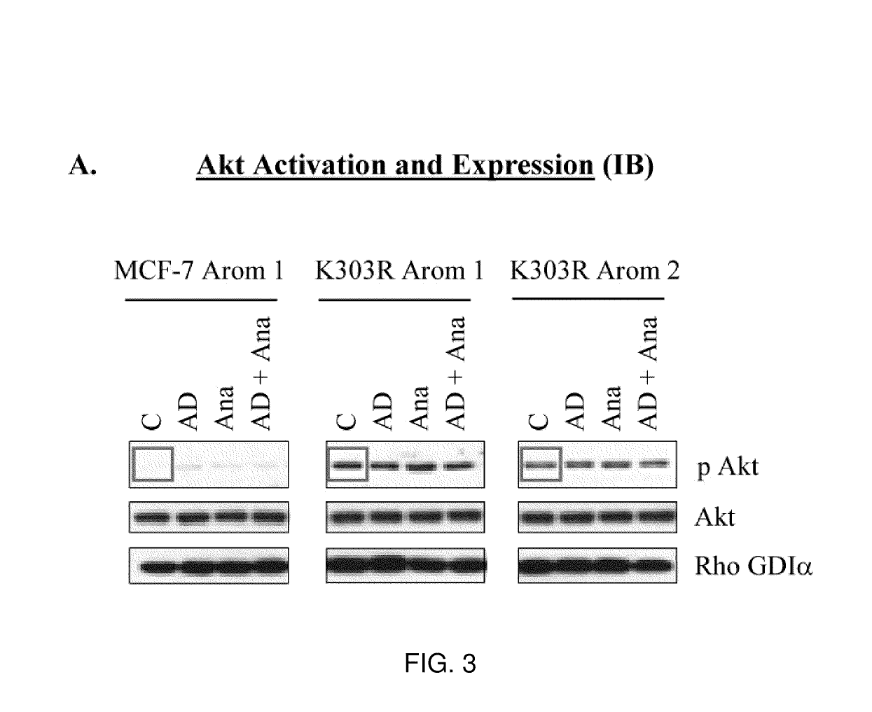

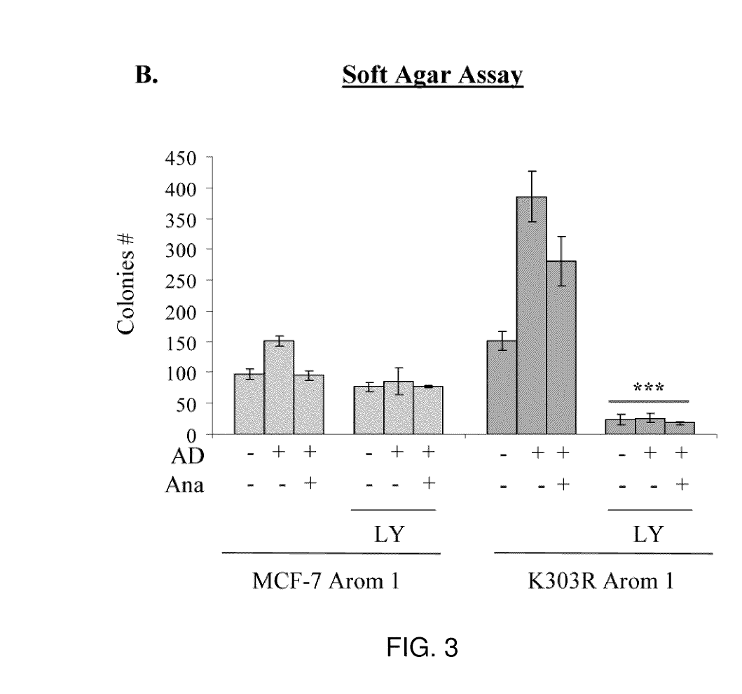

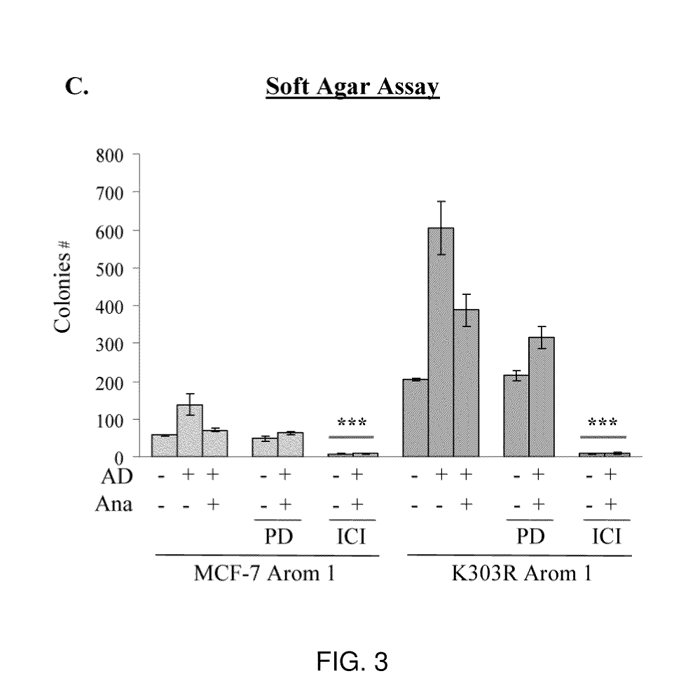

FIG. 3 shows that the PI-3K/Akt pathway is involved in K303R-associated AI^{R} phenotype. In FIG. 3A, MCF-7 Arom 1 and K303R Arom 1-overexpressing cells were treated with vehicle (C), AD (10 nM) and/or Ana (1 μM) for 1 h. Whole cell lysates were analyzed for phosphorylation and expression of Akt by IB analysis. MCF-7 Arom 1-overexpressing cells showed increased levels of pAKT following AD treatment that was reduced by cotreatment with Ana. In contrast, K303R Arom 1 and 2 expressing cells showed higher constitutive levels of Akt kinase phosphorylation that was not affected by Ana treatment. In FIG. 3B, there are effects of the PI3K/Akt inhibitor LY294002 (10 μM) on soft agar growth of MCF-7 Arom 1 and K303R Arom 1-overexpressing cells. Proliferation of K303R Arom 1 cells was completely inhibited by LY294002, whereas MCF-7 Arom 1 cells showed limited effects on growth rate after LY treatment. In FIG. 3C, there are effects of the MEK-1 inhibitor PD98059 (10 μM) and the pure antiestrogen ICI 182,780 (1 μM) on soft agar growth of MCF-7 Arom 1 and K303R Arom 1-overexpressing cells. Antiproliferative effects of PD98059 were not observed on the basal growth of K303R Arom 1 cells and this inhibitor did not reverse the AI^{R} phenotype. ICI 182,780 significantly suppressed the growth of both cell lines, indicating that ERα is important in growth regulation of these cells. In specific embodiments of the invention, the persistence of active PI-3K/Akt is a mechanism of resistance to aromatase inhibitor therapies associated with the K303R mutation.

### Example 5

### K303R Arom Cells Showed an Altered Apoptotic and Proliferative Response

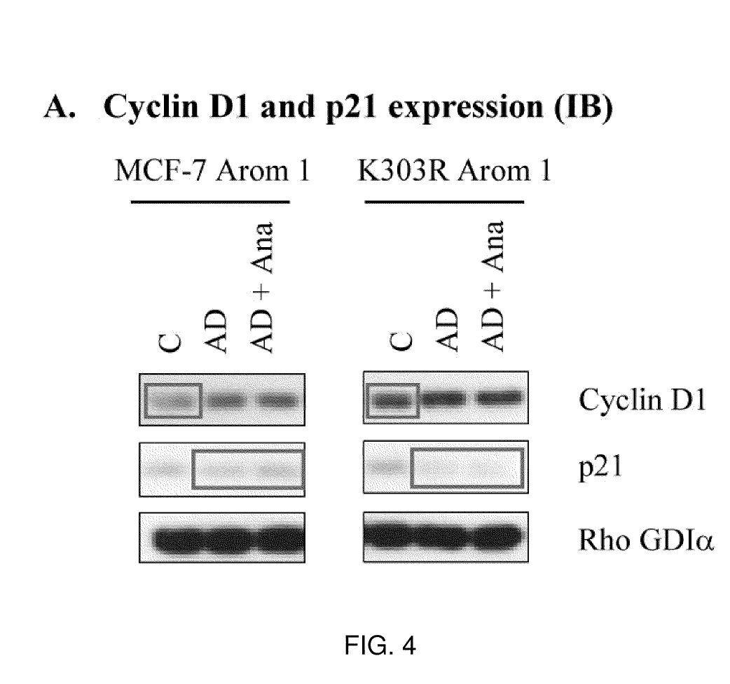

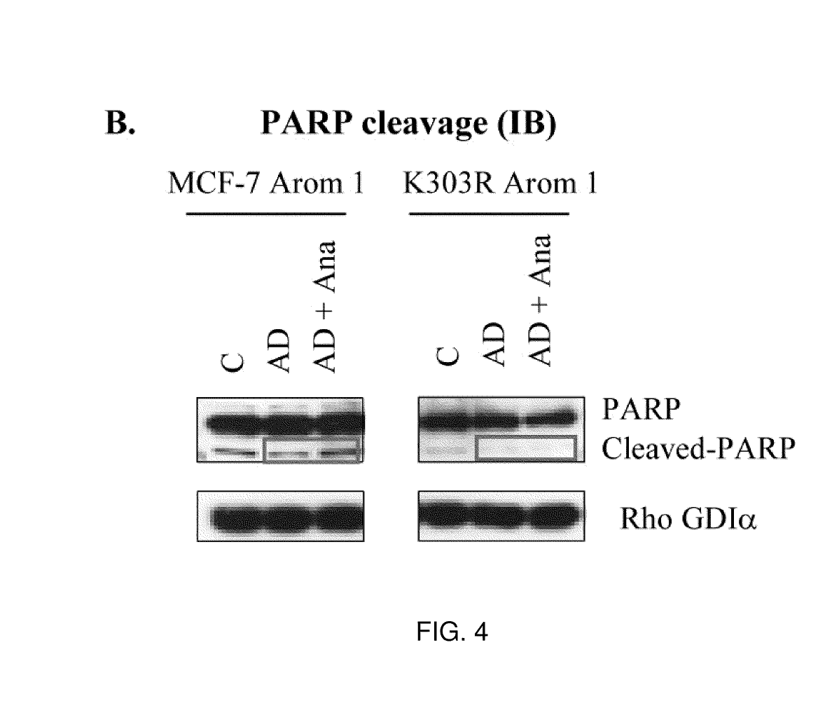

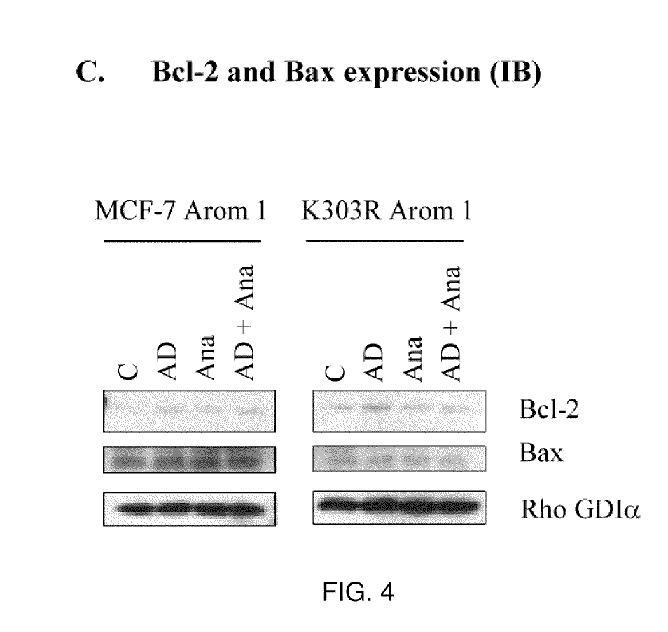

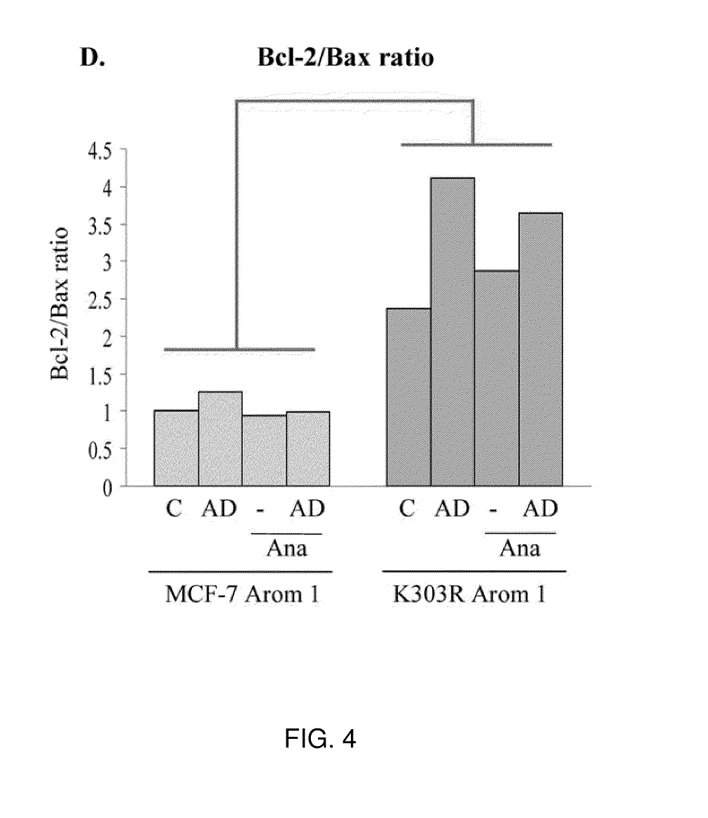

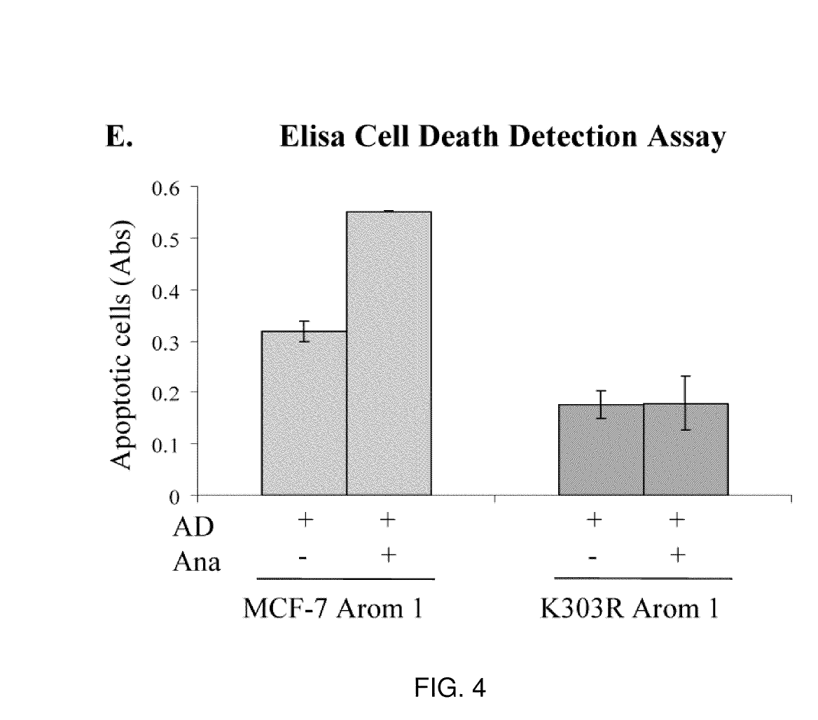

In FIG. 4, K303R Arom Cells showed an altered apoptotic and proliferative response. Steroid-deprived MCF-7 Arom 1 and K303R Arom 1-overexpressing cells were treated with vehicle (C), AD and/or Ana. Whole cell lysates were analyzed for the expression of cyclin D1 and the CDK inhibitor p21 (A), PARP (B), the anti-apoptotic protein Bcl-2 and the pro-apoptotic protein Bax (C) using IB analysis. K303R mutant-overexpressing cells exhibited increased levels of cyclin D1 in basal conditions and decreased levels of the CDK inhibitor p21 after AD and AI treatment. Accordingly, apoptosis was altered with reduction in the basal and stimulated levels of the proteolytic form of PARP (85 kDa), decreased Bax levels, and increased levels of Bcl-2. In FIG. 4D, quantitative analysis of panel C showed an increase in the bcl-2/bax ratio in K303R Arom 1-overexpressing cells, which was further increased with AD and Ana treatments. In FIG. 4E, there is an exemplary Elisa cell death detection assay. AD-stimulated K303R Arom 1 cells exhibited a lower frequency of apoptosis compared to AD treatment in MCF-7 Arom 1 (p<0.05). In addition, Ana treatment induced a significant increase in cell apoptosis in MCF-7 Arom 1 (p<0.005), while no change in apoptotic response was observed in K303R Arom 1 expressing cells. In specific embodiments of the invention, the resistance conferred by the mutation is through effects on cell survival processes.

Thus, the A908G/K303R ERα mutation confers resistance to the non-steroidal AI anastrazole. The Akt pathway plays a role in AI^{R} associated with this mutation, in particular aspects of the invention. The mutation protects the cells from apoptosis induced by an AI, providing breast cancer cells with a potential survival advantage, in certain cases. The results indicate that the mutation is a new predictive marker of response to AIs in breast tumors, and highlight the need for novel approaches toward the development of PI3K/Akt inhibitors for treating patients with tumors resistant to hormone therapy.

### Example 6

### Growth Factor Activation Decreases Responsiveness to Tamoxifen in Cells Expressing the K303R ERα Mutation

Estrogens play a crucial role in regulating breast tumor growth, which is the rationale for the use of antiestrogens, such as tamoxifen (Tam), in women with estrogen receptor (ER)-a-positive breast cancer, however resistance is a major clinical problem. Altered growth factor signaling to the ERα pathway has been shown to be associated with the development of clinical resistance. A previously identified mutation at nucleotide 908 of ERα (A908G) was identified in premalignant hyperplasias and invasive breast cancers. This mutation replaces arginine for lysine at residue 303 (K303R ERα), and confers the ability for enhanced growth in low physiological levels of estrogen. To determine whether the mutation at this site could play a role in resistance, MCF-7 breast cancer cells stably transfected with either wild-type (WT) or K303R mutant ERα were generated. Additive effects of various growth factors (heregulin, epidermal growth factor [EGF], and insulin growth factor [IGF]) were tested in the absence or presence of Tam on cell growth in these cells. The mutation alters Tam response, and growth factor stimulation converted Tam into an agonist in mutant-expressing cells. Tam was less efficient at reducing estrogen-stimulated growth of mutant expressing cell in soft agar assays as well. Tam was also unable to block heregulin-induced soft agar growth of mutant cells, as compared to MCF-7 WT cells. Mutant cells had increased phosphorylated HER2, Akt, and MAPK levels compared to WT cells. Time course studies demonstrated earlier heregulin-induced phosphorylation of these signalling molecules and c-Src in K303R-expressing cells. To examine the effects of growth factors on estrogen-induced genomic activity, transcriptional assays were performed with an AP-1 luciferase gene reporter. EGF and heregulin induced high AP-1 activity, but only in mutant-expressing cells. In a specific embodiment of the invention, the mutation adapts the receptor for enhanced bidirectional cross-talk with growth factor signaling pathways, which then impacts on Tam response. These results indicate that the presence of the A908G ERα mutation is useful as a predictive biomarker of hormonal response in patients whose tumors exploit ERα cross-talk to evade treatment.

### Example 7

### YFP-ERα Expression In Stably Transfected MCF-7 Cells

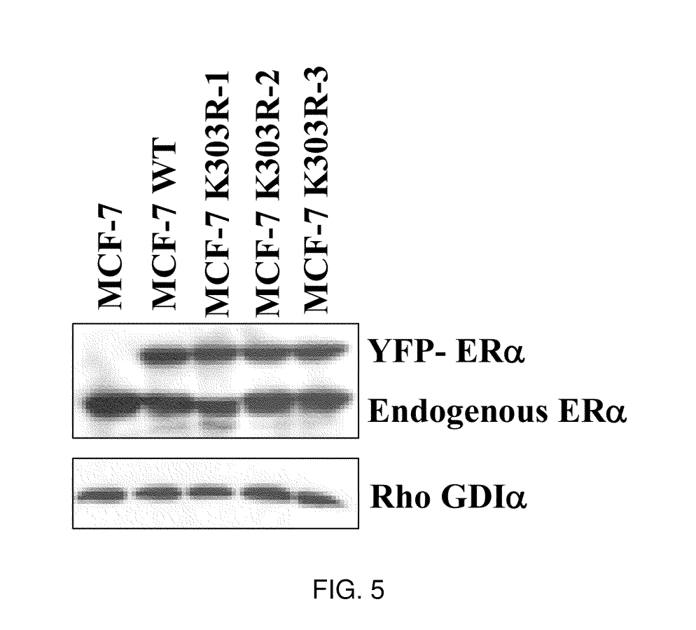

As shown in FIG. 5, MCF-7 cells were stably transfected with yellow fluorescent protein (YFP)— tagged expression constructs. YFP-WT ERα and the mutant YFP-K303R ERα were screened for expression of the exogenous ERα (YFP-ERα), and endogenous ERα by immunoblot analysis. Clones stably expressing YFP-WT ERα and YFP-K303R ERα with similar levels of the exogenous YFP-ERα and endogenous ERα were chosen for further analysis.

### Example 8

### MCF-7 K303R ERα-Expressing Cells Show Altered Sensitivity to Tamoxifen

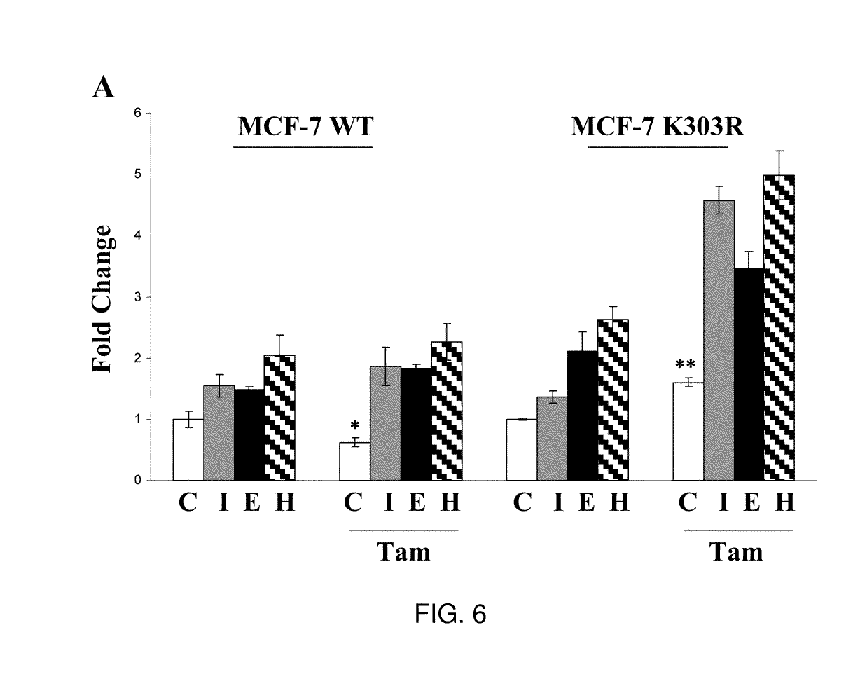

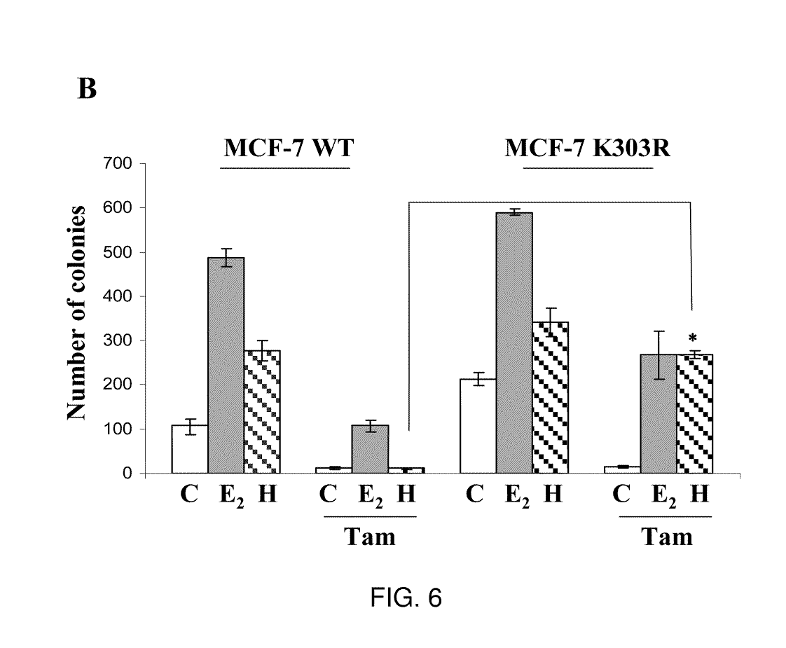

As shown in FIG. 6A, there is an exemplary MTT growth assay. MCF-7 WT or K303R ERα-expressing cells were treated with vehicle (C) or heregulin (H), EGF (E), or IGF-1 (I), with or without Tam. Tam treatment inhibited control growth of WT cells (* p=0.01), but the growth of mutant cells was enhanced by Tam alone (* * p=0.0002). Tam was a super-agonist on the growth of mutant cells when they were stimulated with growth factors. In FIG. 6B, there is an exemplary soft agar growth assay. Cells were serum-starved for 48 h in phenol red-free MEM and plated in 0.35% agarose.

Cells were treated with estrogen (E₂) or heregulin (H) with or without Tam. After 14 days colonies >50 μm diameter were counted. Tam failed to inhibit anchorage-independent growth induced by heregulin in MCF-7 K303R ERα-overexpressing cells, compared to WT (*p=0.0001).

### Example 9

### Growth Factor Signaling is Amplified In MCF-7 K303R ERα— Expressing Cells

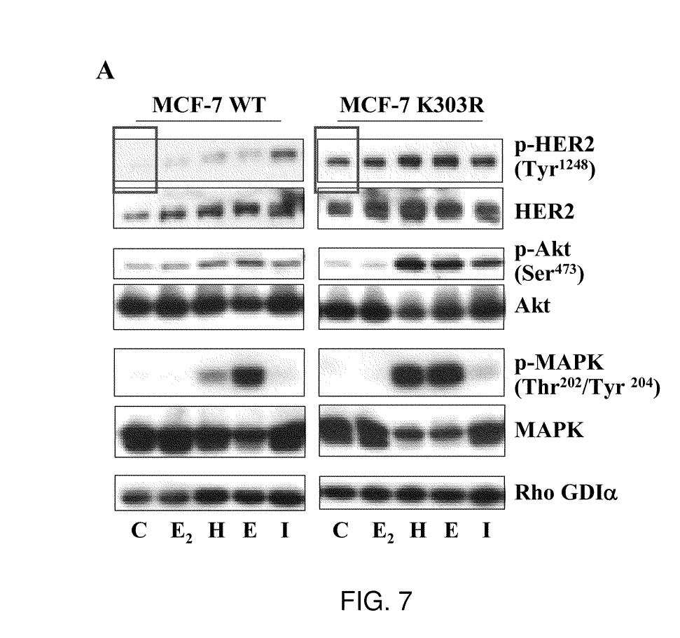

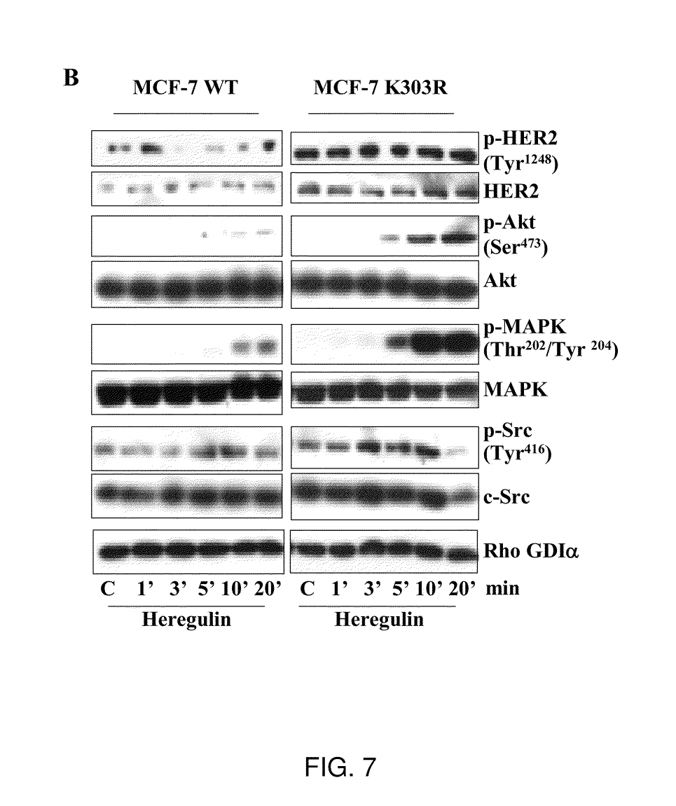

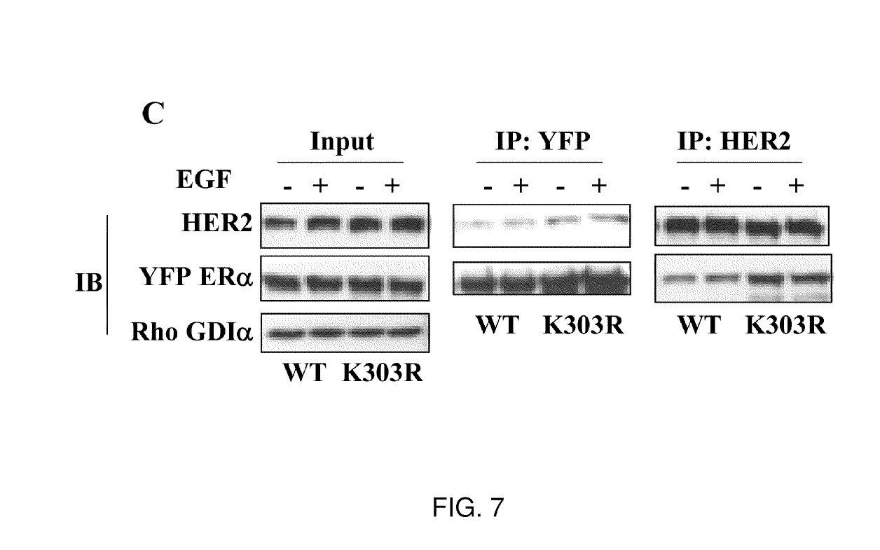

In FIG. 7A, MCF-7 WT or K303R ERα-expressing cells were treated for short term (10 min) with vehicle (C), E₂, heregulin (H), E, or I and extracts were subjected to immunoblot (IB) analysis. Samples were tested for the phosphorylation levels of HER2, Akt, and MAPK; Rho GDIα was used as loading control. K303R mutant-expressing cells exhibited elevated basal levels of pHER2 and total HER2, and increased phosphorylation levels of downstream growth factor signaling molecules AKT and MAPK when compared to WT expressing cells. In FIG. 7B, cells were treated with heregulin for different times and cellular extracts subjected to IB analysis. Time course studies demonstrated earlier heregulin-induced phosphorylation of these signaling molecules and c-Src in K303R ERα-expressing cells. In FIG. 7C, cellular extracts from MCF-7 WT and K303R ERα-expressing cells, treated for 5 min with EGF 100 ng/ml, were subjected to immunoprecipitation using YFP-polyclonal antibody or HER2 polyclonal antibody followed by immunoblot with HER2 and ERα-monoclonal antibodies. The K303R mutant was constitutively associated with HER2.

### Example 10

### AP-1 Activity Induced by Growth Factor In K303R ERα-Expressing Cells

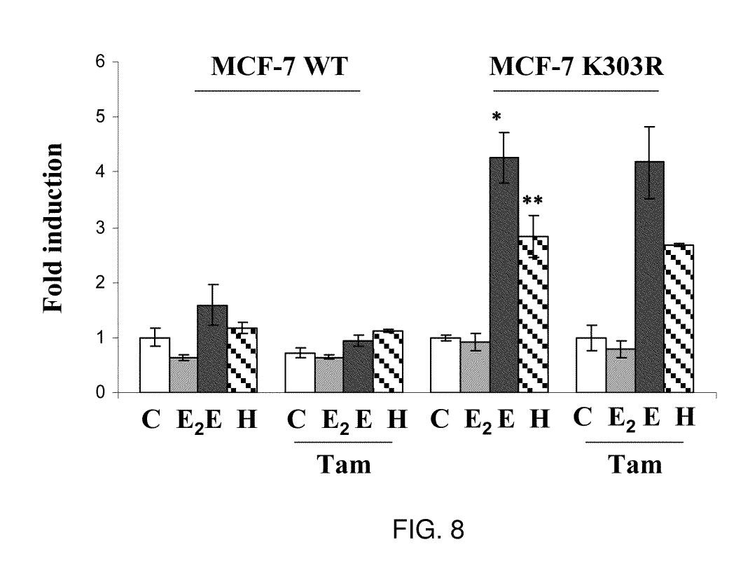

In FIG. 8, AP-1 activity in MCF-7 WT and MCF-7 K303R cells was evaluated using luciferase reporter assays with an AP-1 dependent reporter construct. Cells were treated with estrogen (E₂), EGF 1 (E), or heregulin (H), in the absence or presence of Tam. AP-1 luciferase activity was normalized by β-galactosidase activity to give relative luciferase units. The results are presented as fold induction over control ±SD. Growth factor treatment significantly increased AP-1 activity only in K303R ERα expressing cells and Tam was unable to reverse these effects. It is determined whether enhanced AP-1 activity affects growth factor receptor crosstalk in these cells. * p=0.0003 vs control (C); * * p=0.0028 vs control (C).

### Example 11

### Phosphorylation Status of Serine 305 in the K303R ERα Mutant may be Involved in Enhancement of Growth Factor Signaling

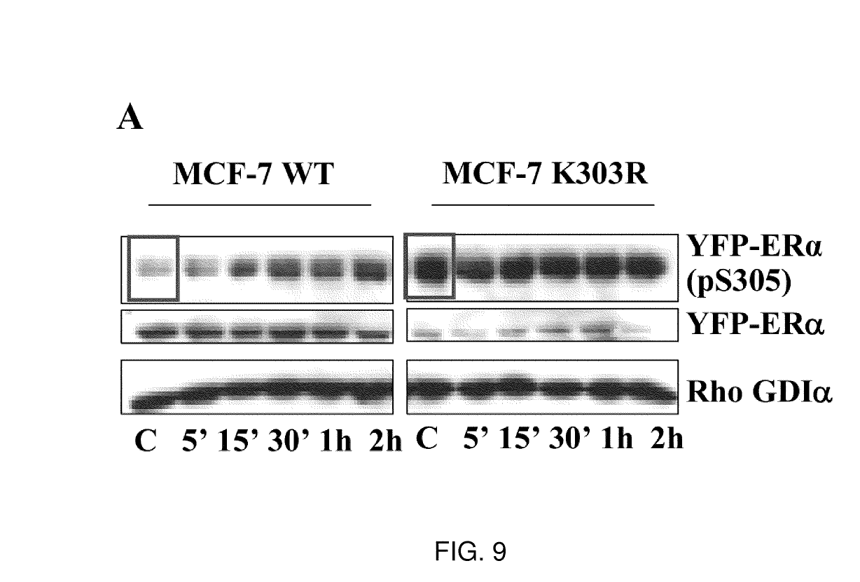

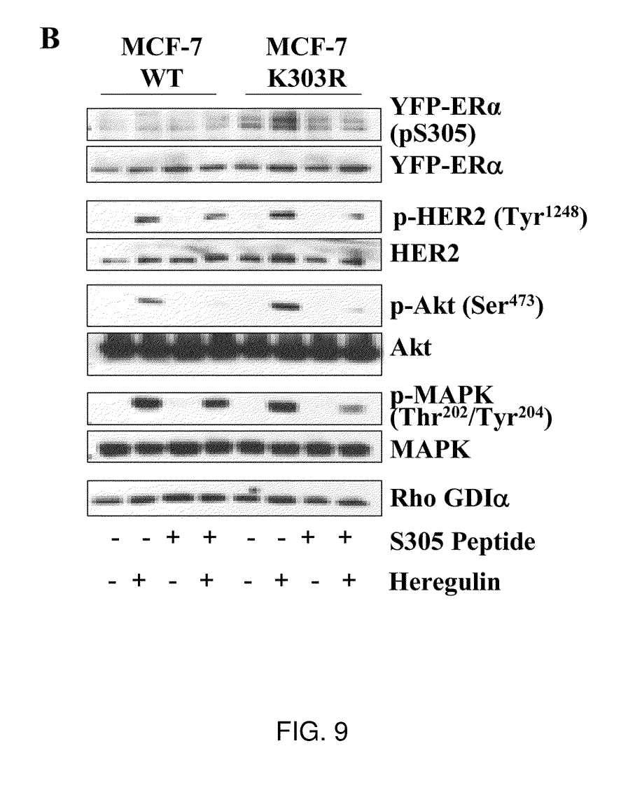

In FIG. 9A, MCF-7 WT and MCF-7 K303R cells were treated for different times with E2. Cellular extracts were analyzed for phosphorylation levels of S305 YFP-WT and YFP-K303R ERα by IB. The K303R mutant receptor showed constitutively elevated levels of pS305 compared to WT ERα. In FIG. 9B, cells were incubated with a S305 blocking peptide for 4 hours and then treated with or without heregulin for 10 min. Levels of phosphorylated (p) S305 YFP-ERα, HER2, Akt and MAPKwere measured by IB. Rho GDIα was used as a control for equal loading and transfer. Addition of the S305 blocking peptide abrogated the activation of downstream signaling molecules, and this effect was more pronounced in mutant-expressing cells . These results indicate that the phosphorylation status of the S305 residue within the hinge domain of ERα plays a role in the up-regulation of growth factors signaling cascade seen in K303R mutant expressing cells, in certain embodiments of the invention.

### Example 12

### Model for Growth Factor Crosstalk with the K303R ERα Mutant Receptor

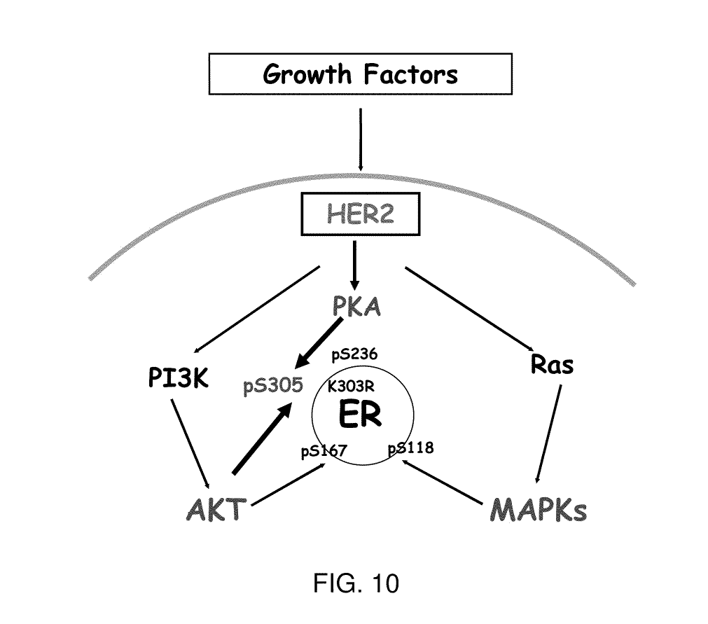

As illustrated in an exemplary model of certain embodiments of the invention, the mutation adapts ERα for enhanced reception of intracellular signal transduction, for example through phosphorylation at S305, for example (FIG. 10).

### Example 13

### Growth Factor-Induced Resistance to Tamoxifen is Associated with a Mutation of Estrogen Receptor a and its Phosphorylation at Serine 305

Exemplary materials and methods are provided herein this example.

### Materials and Methods

Reagents, Hormones and Antibodies

17α-Estradiol (E₂), 4-Hydroxytamoxifen (4-OH), Epidermal growth factor (EGF), Insulin like growth factor-1 (IGF-1), and Heregulin (H) were from Sigma (St. Louis, Mo.). Herceptin was form Genentech (San Francisco, Calif.) Antibodies used for immunoblotting were: ERα (6F11) from (Novocastra, Newcastle, United Kingdom), progesterone receptor (PR) from DAKO (Carpinteria, Calif.), Rho GDIα from Santa Cruz Biotechnology (Santa Cruz, Calif.), total MAPK, total Akt, total c-Src, phosphorylated p42/44 MAPK (Thr²⁰²/Tyr²⁰⁴), Akt (Ser⁴³⁷), c-Src (Tyr⁴¹⁶) from Cell Signaling Technology (Beverly, Mass.), total HER2 from NeoMarker (Fremont, Calif.), phosphorylated HER2 (Tyr¹²⁴⁸), phosphor-ER-S305 from UPSTATE (Temecula, Calif.), and Living Colors™ Full Length polyclonal antibody (Clontech, Mountain View, Calif.).

Cell Culture

MCF-7 breast cancer cells, originally obtained from Dr. Benita Katzenellenbogen (University of Chicago, Urbana, Ill.), were maintained on plastic in minimal essential medium (MEM) (Invitrogen) supplemented with 10% fetal bovine serum (FBS; Summit Biotechnology, Fort Collins, Colo.), 0.1 nmol/L nonessential amino acid, 2 mmol/L L-glutamine, 50 units/ml penicillin/streptomycin, at 37° C. with 5% CO₂/95% air. Hela cells were obtained form American Type Culture Collection (Manassas, Va.), and were maintained in the same media. Generation of the yellow-fluorescent protein (YFP)-tagged expression constructs, YFP-WT ERα and YFP-K303R ERα, has been previously described (Cui et al., 2004). MCF-7 cells were stably transfected using Fugene according to the manufacturer's instructions (Roche, Indianapolis, Ind.), and individual clones were isolated and expanded with G418 selection. In some experiments we used a pool of stably transfected cells selected for one week with G418 antibiotic. Stably transfected clones were screened for expression of exogenous and endogenous ERα using immunoblot analysis.

Quantitative image analyses by high throughput microscopy (HTM)

PRL-Hela is a cell line specifically engineered for the single cell study of ER function (Sharp et al., 2006). PRL-Hela cells contain multiple genomic integrations of a replicated prolactin (PRL) promoter/enhancer. The multiple integrations (PRL array) are spatially confined and are visualized by the accumulation of fluorescently tagged ERα. PRL-Hela cells transiently expressing YFP-WT ERα or YFP K303R ERα, pretreated with forskolin (10 μM) for 15 minutes, and then treated with increasing concentration of E₂ or Tam for 30 minutes, were fixed and DAPI-stained as previously reported (Sharp et al., 2006). The cells were imaged using the Cell Lab IC 100 Image Cytometer (Beckman Coulter, Fullerton, Calif.) with a Nikon 40× Plan S flour 0.90 NA objective. Two channels were imaged: channel 0 (DAPI stain) was used to find the focus and nuclei, when channel 1 was used to image YFP ERα. A proprietary algorithm (GPRC) developed at Beckman Coulter was used to identify and quantify the YFP-ERα-targeted PRL array. The parameters for the GPRC algorithm were: object scale=30 and minimum peak height=10. Foci identified by the GPRC algorithm were masked. The area of the mask in pixels was the measure of PRL array size. Channel 1 was offset 2 μm from DAPI focused for cells in all treatment conditions. After image acquisition and application of the GPRC algorithm, the total cell populations for each treatment were progressively filtered (gated) using the same criteria. Nuclei clusters, and mitotic cells were filtered from the total cell population using an intersection of DNA content and DNA clusters gates. In addition, low YFP ERα expression and low aggregate number gates were generated and applied to produce the final cell population to be analyzed. From the final population of cells, the array size was determined using the GPRC mask (Berno et al., 2006). The images and masks were visually inspected for accuracy. Unpaired students t-test assuming equal variance were performed to determine statistical significance (two-tailed, p<0.05).

Immunoprecipitation and Immunoblot Analysis

Cells were starved in phenol red free MEM with 5% charcoal-stripped FBS for 48 h and treated as indicated before lysis [50 mmol/L Tris-HCl (pH 7.4), 150 mmol/L NaCl, 2% NP40, 0.25% deoxycholic acid, 1 mmol/L EDTA, 1 mmol/L Na₃VO₄, and 1:100 protease inhibitor cocktail; Calbiochem]. For coimmunoprecipitation experiments, we used 1 mg of total cellular protein and a 1:200 dilution of anti-Living colors Full Length polyclonal antisera (Clontech) that recognizes native and denatured forms of recombinant YFP fusion proteins expressed in mammalian cells, and 2 μg of HER2 polyclonal antisera over night, followed by protein A/G precipitation with rotation at 4° C. for 2 h. Immunoprecipitated proteins were washed thrice with lysis buffer. Equal amounts of cell extract and immunoprecipitated proteins were resolved under denaturing conditions by electrophoresis in 8% to 10% polyacrylamide gels containing SDS (SDS-PAGE), and transferred to nitrocellulose membranes (Schleicher & Schuell, Keen, N.H.) by electroblotting. After blocking the transferred nitrocellulose membranes were incubated with primary antibodies overnight at 4° C., with secondary antibodies goat anti-mouse or goat anti-rabbit antisera (1:3000; Amersham Biosciences; Piscataway, N.J.) for 1 h at room temperature and developed with enhanced chemioluminescence reagents (Alpha Innotech, San Leandro, Calif.).

Anchorage-Independent Soft Agar Growth Assays

Cells (5000 per well) were plated in 4 ml of 0.35% agarose with 5% charcoal-stripped FBS in phenol red-free MEM, in a 0.7% agarose base in six-well plates. Two days after plating, media containing control vehicle or hormonal treatments (E₂ 1 nM, heregulin 2 ng/ml, with or without Tam 100 nM) was added to the top layer, and the appropriate media was replaced every two days. In some experiments a pool of stably transfected cells were treated with heregulin with or without herceptin (10 μg/ml). After 14 days, 150 μl of MTT was added to each well and allowed to incubate at 37° C. for 4 h. Plates were then placed in 4° C. overnight and colonies >50 μm diameter from triplicate assays were counted. Data are the mean colony number of three plates and representative of two independent experiments analyzed for statistical significance (p<0.05) using a two-tailed student's Test, performed by Graph Pad Prism 5 (GraphPad Software, Inc., San Diego, Calif.).

Blocking Peptide Delivery

A blocking peptide of 13 residues (IKRSKKNSLALSC; SEQ ID NO:14) from the sequence (residues 298-310) surrounding the S305 residue (in bold) of the human ERα was transferred into cells using a cationic amphiphile molecule, PULSin™ delivery reagent (Polyplus transfection, Illkirch, France), as suggested by manufacturer. Briefly, cells were plated in a 6-well plate with regular growth media, and then starved for 48 h in a phenol red free MEM with 5% charcoal stripped FBS. After starvation cells were washed with PBS to remove all traces of serum, and fresh phenol-red free media without serum was added. The mixture containing the S305 blocking peptide (4 μg/well) diluted in 200 μl of 20 nM Hepes, and 16 μl of PULSin™ was incubated for 15 min at room temperature, and then added to the cells. The media was changed after 4 hours of incubation, cells were treated as indicated, and cellular extracts were prepared. Delivery of R-phycoerythrin was used as a positive control and live cells were observed by fluorescence microscopy after 4 h.

### Ligand-Independent Signaling to the K303R ERα Mutant Reduces Tam Sensitivity

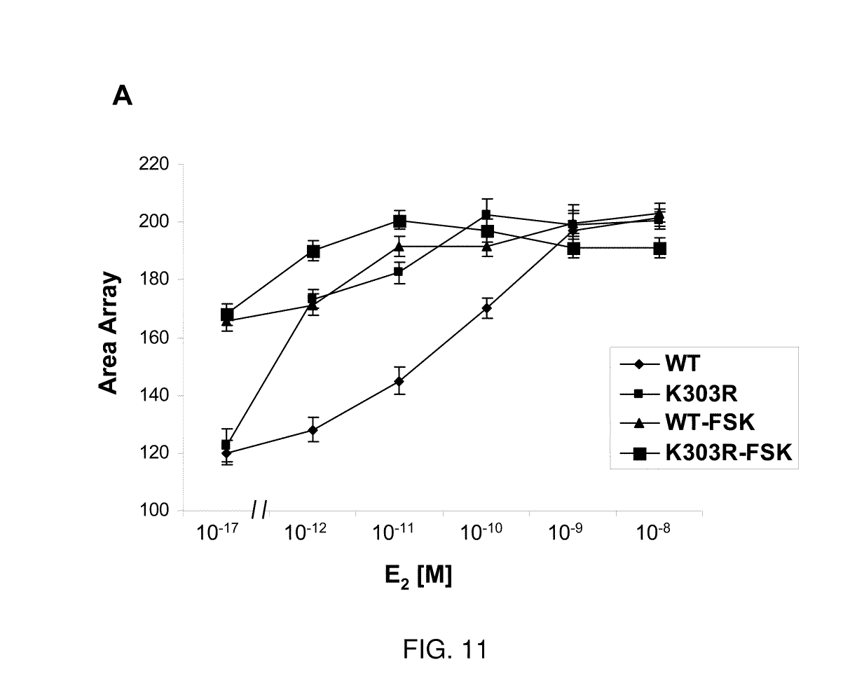

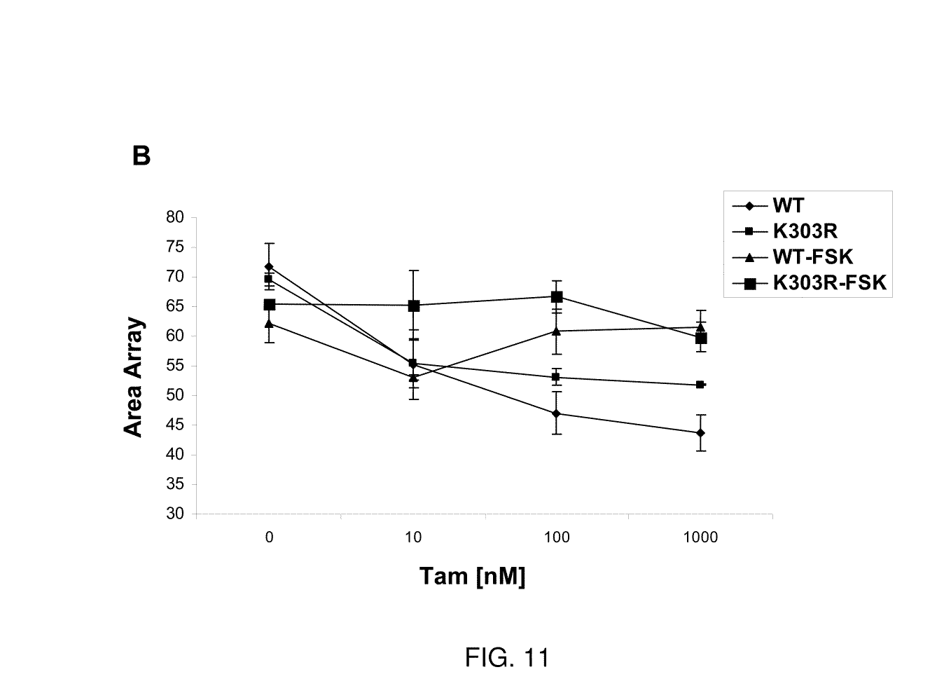

It has been previously shown that K303R ERα mutant-overexpressing cells display enhanced ligand-independent activity when stimulated with cyclic AMP in part because the mutation generates a more efficient substrate for PKA-mediated signalling (Cui et al., 2004). It is well known that ligand-independent signalling can influence cellular responsiveness to Tam (Smith et al., 1993). For instance, PKA-mediated phosphorylation of ERα at S305 allows the antagonist Tam to behave as an agonist, which then results in ERα-dependent transactivation (Michalides et al., 2004). Here the inventor has utilized the stable cell line PRL-Hela containing a multi-copy integrated prolactin enhancer/promoter DNA array that is responsive to ER such that when the receptor is expressed as a YFP-fusion protein, the integration site can be easily visualized (PRL-array) (Sharp et al., 2006). High throughput microscopy has been used to identify ligand-independent changes in the size of the PRL-array which is an indicator of the chromatin condensation status at the promoter (Berno et al., 2006). Arrays rapidly decondense after E₂ treatment or condense after anti-estrogen treatment (both within minutes). Thus, in PRL-Hela, array size is an indicator of receptor-mediated transcription function in response to different treatments allows direct and live observation of ER-dependent chromatin remodelling. Therefore using a mammalian-based, stably transfected prolactin promoter chromosomal array system, live cell dynamics were analyzed on an estrogen-responsive promoter to visualize chromatin remodelling induced by E₂ and Tam. PRL-Hela cells were transiently transfected with YFP tagged-ERα WT or the YFP-ERα K303R mutant receptor expression vector; E₂ (FIG. 11a) and Tam's (FIG. 11b) effect on chromatin remodelling in the presence of forskolin, an activator of PKA signalling, were assayed. Using high through-put microscopy, the amount of chromatin condensation was quantified (Berno et al., 2006). 24 h after transfection cells were pretreated with forskolin (FSK), and then treated with E2 or Tam at different doses, as indicated, for 30 minutes. Over 60 nuclei were analyzed to determine the size of the PRL-reporter array. Vehicle control, unliganded receptors were similarly condensed (FIG. 11b). In Hela cells expressing the WT receptor, estrogen treatment increased array size in a dose-dependent manner in estrogen concentrations between 10⁻¹² M and 10⁻⁹ M. Mutant-expressing cells showed a linear dose-response in the range of 10⁻¹⁷ M through 10-11 M of estrogen. Indeed, mutant ERα expressing cells exhibit a much lower EC50 (3×10⁻¹³ M) compared with WT ERα-expressing cells (5.1×10⁻¹¹ M). These data demonstrate that K303R mutant-expressing cells are hypersensitive to estrogen and form larger arrays in the presence of low estrogen concentrations. With forskolin treatment, WT and mutant ERα cells stimulated with estrogen both displayed significantly increased array sizes. WT ERα-expressing cells demonstrated a one-log reduction in the EC50 (4.2×10⁻¹² M ±0.0038 compared to 5.1×10⁻¹¹ M ±0.0132 in the absence of forskolin) and a significantly increased average array size (FIG. 11a). Mutant-expressing cells also displayed a significantly increased average size, but without a significant change in the EC50 (8×10⁻¹³ M ±612.5 compared to 3×10⁻¹³ M ±0.0004 in the absece of forskolin). Thus, forskolin increased average array size, and reduced the EC50 of WT ERα cells thus enhancing estrogen sensitivity, whereas forskolin treatment of mutant-expressing cells only increased the array size, suggesting that these cells displayed an inherent hypersensitivity to estrogen. As shown in FIG. 11b, Tam treatment decreased the array decondensation in WT ERα-expressing cells, however the mutant receptor was significantly less responsive to Tam (p<0.0001). Forskolin treatment blocked Tam-induced promoter condensation, and the mutant receptor array size was not affected by physiological levels of Tam (100 nM) in the presence of forskolin. All together these results indicate that hormone-independent kinase signalling to the mutant receptor K303R ERα confers resistance to antiestrogen treatment in breast cancer cells, in particular aspects of the invention.

### MCF-7 K303R-ERα Mutant Expressing Cells Exhibit Altered Growth Factor Signalling

To further explore ligand-independent activation of signalling in mutant-expressing breast cancer cells, MCF-7 ERα-positive human breast cancer cell lines were developed overexpressing either WT or the K303R ERα mutant. The mutant receptor was introduced into parental WT ERα-expressing cells to simulate the situation in invasive human breast tumors, where WT receptor is most frequently co-expressed along with the mutant (Herynk et al., 2007). To differentiate the exogenously expressed receptor, the vector was tagged with YFP. Stably transfected clones were screened for expression of ERα using immunoblot analysis (FIG. 5). Parental MCF-7 cells are shown along with one clone stably expressing YFP-WT, and three clones expressing YFP-K303R ERα (MCF-7 K303R-1-3). Clones with similar levels of the exogenous and endogenous receptor were chosen for further analysis.

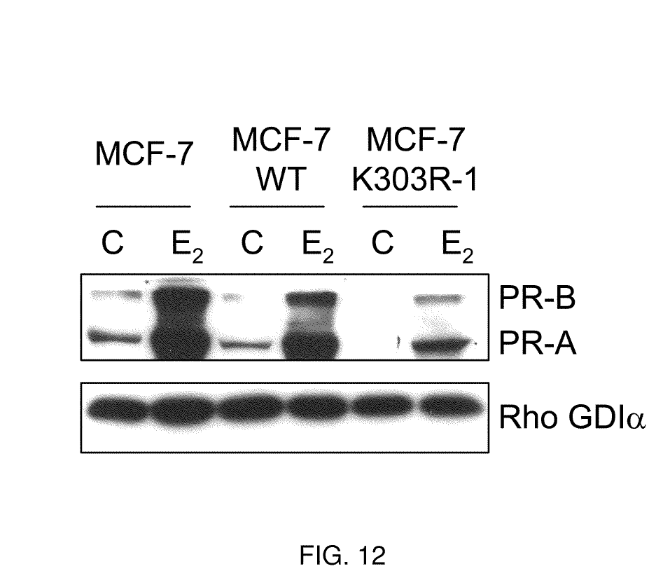

It is well accepted that the response to endocrine therapies in human breast cancer patients correlates with ERα and progesterone receptor (PR) levels. Several studies have shown that patients with ERα/PR-positive breast cancers derive greater benefit from adjuvant hormonal therapy than those patients whose tumors lack PR (Bardou et al., 2003; Ravdin et al., 1992; Elledge et al., 2000], however it must be noted that other studies have not always found this result (Goss et al., 2000). It has been hypothesized that high growth factor signalling activity in breast cancers may be associated with decreased PR levels (for a review see Cui et al., 2005), and elegant studies have shown that up-regulation of pMAPK Erk1/2 leads to a loss of PR via degradation by the 26S proteasome (Lange et al., 2000). In clinical samples the inventor has previously reported a borderline significant inverse correlation between the presence of the K303R ERα mutation and the PR-B isoform (Herynk et al., 2007), and a reduction in PR-B levels was associated with a poorer response to endocrine therapy (Hopp et al., 2004). Therefore, the inventor first evaluated the levels of PR-A and B in parental MCF-7 cells and two stably transfected clones (FIG. 12). Parental MCF-7 cells expressed both PR isoforms, with PR-A as the predominant form in these cells. PR is a known estrogen-induced gene, and as expected we saw higher levels of both PR-A and B in MCF-7 cells after estrogen (E₂) treatment. In comparison, MCF-7 WT-overexpressing cells exhibited slightly less PR induction with E₂. In contrast, under control, basal conditions, K303R ERα mutant-overexpressing cells demonstrated much lower levels of both PR isoforms, and less were induced with E₂ treatment. This result indicates that growth factor signals are altered in the mutant-overexpressing cells, in certain aspects of the invention.

To test for altered intracellular signalling, the effects of short-term treatments with E2, heregulin (H), EGF (E), and IGF-1 (I) were examined on phosphorylation levels of downstream growth factors signalling components, such as HER2, Akt, and MAPK using immunoblot analysis (FIG. 7c). Cells were maintained under estrogen-depleted conditions for 2 days, and then treated for 10 minutes with E2 or different growth factors as indicated, and cellular extracts were prepared. MCF-7 K303R mutant cells showed constitutively higher levels of phosphorylated HER2 as well as total HER2, compared to MCF-7 WT ERα-overexpressing cells (FIG. 7c). To characterize the mechanism associated with higher levels of total HER2 in the mutant cells, the expression level of HER2 mRNA in different clones of mutant ERα expressing cells was analyzed by Real-Time polymerase chain reaction (qPCR); there was no difference in the mRNA level of HER2 between WT and K303R ERα expressing cells. This result indicates that post-translational modification and increase in protein stability are involved in the HER2 up-regulation that was found in mutant ERα-expressing cells, in certain embodiments of the invention. Treatment with E2, heregulin, EGF, and IGF-1 induced higher levels of pHER2 in mutant-overexpressing cells, but only a small induction was seen in MCF-7 WT cells with low endogenous levels of HER2. Treatment with heregulin or EGF led to increased phosphorylation of the downstream signalling molecules Akt and MAPK in mutant-overexpressing cells compared with MCF-7 WT cells. IGF-1 treatment also induced enhanced phosphorylation of Akt, but increased levels of phosphorylated MAPK were only detected in K303R ERα mutant-overexpressing cells. In both cell lines, no enhancement of Akt and MAPK phosphorylation was seen after estrogen treatment.

To investigate if the kinetics of growth factor signalling are altered in mutant-expressing cells, a time-course study was performed with heregulin treatment in WT ERα and K303R ERα mutant cells. After two days of starvation, cells were treated for 1, 3, 5, 10, or 20 minutes with heregulin, and then lysed as described elsewhere herein. As shown in FIG. 7B, a rapid response to heregulin was seen within 1′ in MCF-7 WT cells after activation of phospho-HER2, and phospho-AKT/pMAPK within 10-20′. In contrast, mutant-expressing cells exhibited enhanced activation of pHER2, pAKT, and pMAPK. Compared with WT-expressing cells, these molecules were all stimulated at earlier time points in mutant cells; heregulin induced phosphorylation of MAPK and Akt by 5 minutes, but levels of these phosphorylated kinases were not detectable at this time-point in WT ERα cells. The non receptor tyrosine kinase c-Src also exhibited an earlier time-point of induction (at 3 min), and higher phosphorylation levels at the activating c-Src tyrosine residue 416 (Tyr416) was seen in K303R ERα cells. These collective data demonstrate that the mutant-overexpressing cells are hypersensitive to growth factor signal transduction, and indicate that the mutant receptor could impact on ligand-independent signalling pathways commonly known to be employed to evade anti-hormonal therapeutic strategies in breast cancer.

Previous reports have shown that ERα and growth factor pathways can interact at different levels, through a direct association or complex formation of ERα with key signalling molecules such as c-Src, Shc, and the p85 cc regulatory subunit of PI3K (Migliaccio et al., 2005; Wong et al., 2002; Song et al., 2002; Sun et al., 2001). C-Src family tyrosine kinases are involved in signalling of a number of different growth factor receptors, including EGFR/HER2, in breast cancer cells. In previous work the inventor has reported that MCF-7 cells stably expressing the K303R ERα mutant receptor exhibited increased c-Src kinase activity and c-Src tyrosine phosphorylation, when compared with WT ERα-expressing cells (Herynk et al., 2006). Therefore it was next addressed whether the mutation might alter the ability of the receptor to bind with the HER2 tyrosine kinase receptor, which is expressed at higher levels in the mutant cells (FIG. 7A). To evaluate potential protein-protein interactions between WT and mutant receptors with HER2 in the model system, MCF-7 cells overexpressing WT or the mutant for 5′ were treated with EGF 100 ng/ml and then lysates were prepared (FIG. 7C). Equal amounts of protein were immunoprecipitated with either anti-YFP antisera or anti-HER2 antisera followed by immunoblot for HER2 and YFP-ERα. As shown in FIG. 7C similar amounts of input from the whole-cell lysates were used. In the absence of treatment, both WT ERα and HER2 were in a protein complex; EGF treatment slightly increased the amount of the protein in this complex. The levels of mutant receptor bound to HER2 under basal conditions were higher, and EGF treatment did not further increase the amount of receptor in the complex. The bottom panel shows that equal amounts of YFP-ERα and HER2 were immunoprecipitated under all conditions tested. These results indicate that the K303R ERα mutant may be constitutively associated with HER2, which might be involved in the enhanced bidirectional crosstalk discovered between ERα and growth factor receptor signalling pathways.

### Tamoxifen Fails to Inhibit Anchorage-Independent Growth Induced by Heregulin in MCF-7 K303R ERα-Overexpressing Cells

It is already appreciated that altered crosstalk between several receptor tyrosine kinases and ERα may contribute to endocrine resistance (Schiff et al., 2006), and it has been shown that elevated levels of HER2 contribute to resistance in a tamoxifen-resistant subline of MCF-7 cells compared to unselected parental cells (Nicholson et al., 2001). Since the mutation conferred enhanced heregulin-mediated signalling, it was examined whether this confers an altered response to antiestrogen therapy. To test this question an anchorage-independent growth assay was performed using two exemplary ERα-overexpressing models. Cells were plated in soft agar and then treated with estrogen (E₂, 1 nM) or heregulin (H, 2 ng/ml) in the presence or absence of tamoxifen (Tam, 100 nM). After 14 days of growth colonies >50 μM in diameter were counted (FIG. 6B). Control (C) basal growth of mutant-expressing cells was higher compared to WT-expressing cells (p=0.001); as expected treatment with estrogen as well as heregulin increased the number of colonies in both cell lines. Tam treatment induced a significant reduction in the number of colonies in MCF-7 WT cells under control conditions, and was able to inhibit growth of cells stimulated with estrogen or heregulin (reduction in colonies 78% and 96%, respectively). In contrast Tam was less efficient at inhibiting estrogen or heregulin-stimulated proliferation in mutant-overexpressing cells (reduction were only 54% and 21%, respectively). Thus, response to Tam was greatly affected by the enhanced growth factor signalling in mutant cells; this enhanced sensitivity prevented the major antagonistic activity of tamoxifen on the proliferation of mutant-expressing cells.

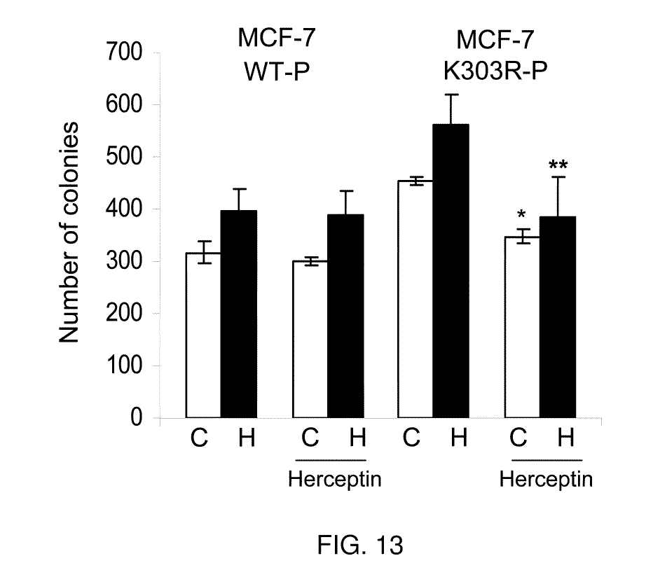

Next, it was examined whether the HER2 pathway found to be up-regulated in the mutant-expressing cells was responsible for the higher growth of mutant cells compared to WT ERα cells. Anchorage-independent growth of either mutant or WT ERα cells after herceptin treatment was evaluated. Herceptin is a humanized monoclonal antibody directed against the extracellular domain of HER2, and was developed as an agent to inhibit the growth of HER2-overexpressing tumor cells (Vogel et al., 2002; Romond et al., 2005). Cells were plated in soft agar and then treated with heregulin in the presence or absence of herceptin (10 μg/ml). After 14 days of growth, colonies were counted (FIG. 13). As expected since HER2 levels are low in WT cells, herceptin had no effect on their growth either under basal conditions or with heregulin treatment. In contrast, herceptin significantly inhibited anchorage-independent growth of K303R mutant cells in control untreated conditions, and with heregulin treatment (*p=0.0002 vs control; **p=0.03 vs heregulin treated cells). These data confirm that the HER2 pathway is responsible for the higher constitutive growth of mutant-overexpressing cells rendering these cells more sensitive to the inhibitory effect of this selective HER2-targeted agent, in certain embodiments of the invention.

### Phosphorylation at Serine Residue 305 (S305) of ERα K303R Mutant is Involved in Growth Factor Signalling Up-Regulation

ERα is a known target of several post-translational modifications, such as phosphorylation, sumoylation, and acetylation (Fuqua et al., 2000; Likhite et al., 2006; Kim et al., 2006; Sentis et al., 2005). For instance, receptor phosphorylation, which regulates receptor affinity, coregulator protein binding, and transcriptional activity, can be induced in the absence of ligand via crosstalk with various signal transduction pathways (Lannigan et al., 2003). It was previously reported that the K303R ERα mutant is a more efficient substrate for phosphorylation by PKA at S305 that enhanced hormone sensitivity and stimulated cellular growth (Cui et al., 2004). In certain embodiments of the invention, the phosphorylation status of S305 in the mutant receptor controls receptor activity and is a conduit for enhanced downstream crosstalk with growth factor signalling networks. To examine this, the phosphorylation status of the S305 residue in either WT ERα or K303R mutant cells was evaluated after estrogen treatments between 5 minutes to 2 hours. Cellular extracts were subjected to immunoblot assay using a specific anti-phospho-S305 ERα antibody (FIG. 9a). Estrogen treatment upregulated the phosphorylated levels of S305 after 15′ in YFP-WT-expressing cells and this level remained elevated at 2 hours. In contrast, K303R ERα mutant-expressing cells exhibited elevated levels of pS305 under basal control conditions, and this elevated phosphorylation remained constant with longer estrogen treatments. Since, it is well established that ERα can be activated in a ligand-independent manner by MAPK (Karas et al., 1998) at serine 118 (S118), the levels of phospho-S118 were evaluated in WT ERα or K303R ERα overexpressing cells under the same conditions described above. No significant changes in S118 phosphorylation patterns were detectable between the two cell lines. These results indicate that constitutively higher phosphorylation of S305 in the mutant receptor plays a role in the ligand-independent activation of the receptor itself, in certain aspects of the invention. In particular embodiments, this enhanced S305 phosphorylation within the mutant plays a role in the observed up-regulation of growth factor signalling cascades seen in these cells as well.

To further examine this, immunoblot analysis was performed to evaluate the phosphorylation levels of a number of growth factor signalling components after incubation with an S305 blocking peptide. To block phosphorylation at S305 a peptide (S305 peptide, residues 298 to 310) was delivered to the cells. After peptide delivery, the cells were subjected to short term treatments (10 min) with heregulin and then growth factor signalling molecules were analyzed by immunoblot analysis (FIG. 9b). Heregulin enhanced S305 phosphorylation in K303R mutant-overexpressing cells, but had no effect WT receptor. Phosphorylation of the mutant receptor was abrogated by the S305 peptide. Addition of the S305 blocking peptide also inhibited heregulin-induced phosphorylation of HER2, Akt, and MAPK in both cell lines. Interestingly, reduction in phospho-HER2 and MAPK levels were more pronounced in mutant-expressing cells compared with WT, indicating that the mutant cells were more sensitive to the inhibitory effect of S305 blocking peptide. These data indicate that phosphorylation of the S305 residue are crucial in mediating enhanced crosstalk between HER2 and mutant ERα, and indicate that phosphorylation blockade is a therapeutic strategy to block mutant function, in certain embodiments of the invention.

### Significance of Certain Embodiments of the Invention

Despite the improvements in the efficacy of hormonal therapies for the treatment of breast cancer patients with ER-positive tumors, de novo and acquired resistance remain major clinical problems that limit the efficacy of these therapies. In most cases, ERα remains essential to the problem of hormone resistance due to its intimate crosstalk with growth factor signalling pathways (Encarnacion et al., 1993; Brunner et al., 1993). In this invention, it was shown that expression of K303R ERα mutant in ERα-positive MCF-7 breast cancer cells confers a decreased sensitivity to tamoxifen treatment in the presence of growth factor stimulation. Furthermore, this naturally-occurring mutant is constitutively phosphorylated at S305, and shows an enhanced bidirectional crosstalk with the HER2 signalling pathway.

The A to G somatic mutation of ERα at nucleotide 908 (A908G) was previously identified in about 30% of premalignant breast lesions, but at a higher frequency (50%) in invasive breast tumors (Fuqua et al., 2000; Herynk et al., 2007). The mutation was found to be associated with biologic measures of poor outcome, including elevated HER2, larger tumor size and axillary lymph node positivity. To date no other somatic ERα mutation has been identified in more than a few invasive breast cancers (Herynk and Fuqua, 2004), making this mutation novel. Recently, in a study using a population-based, case-control study design, the A908G mutation was detected, but at a low frequency (7%), in invasive breast tumors (Conwy et al., 2007), confirming our identification of the mutant in cancer. It was previously demonstrated that the exogenous expression of the K303Rα mutant in MCF-7 breast cancer cells conferred a hypersensitive growth in very low physiological levels of estrogen (10⁻¹² to 10⁻¹¹ M) (Fuqua et al., 2000). In the present invention the inventor focused on the K303R ERα mutation and its potential role in modulating hormonal response in breast cancer cells, and on the molecular pathways that could be involved in its hormone action.

The studies showed that growth factor signalling pathways were up-regulated in K303R ERα-expressing cells. In particular, MCF-7 mutant ERα expressing cells showed constitutively higher levels of total and phosphorylated HER2, the tyrosine kinase receptor that belongs to the epidermal growth factor receptors family. It is well known that HER2 catalytic activity can amplify the signal of other erbB family receptors by the formation of HER2-containing heterodimers, which increases ligand binding affinity and receptor stability (Worthylake et al., 1999; Wang et al., 1998). Moreover, it has been shown that HER2/neu, the mouse homolog of HER2, is able to multimerize, be phosphorylated, and thus activated when present at high density on the cell surfaces (Samanta et al., 1994). Both mechanisms result in the amplified activation of downstream signalling cascade, such as Akt and MAPK, which are involved in cell survival and proliferation.

The peptide growth factors heregulin and EGF strongly enhanced phosphorylation of the two major downstream signalling cascades Akt and MAPK, in mutant-expressing cells compared to WT ERα-expressing cells. Furthermore, analysis of rapid kinetics showed that many of these downstream molecules, as well as Src non receptor tyrosine kinase, were stimulated at earlier time points in mutant-expressing cells. The rapid responses of these downstream kinase cascades to heregulin indicate that the presence of K303R ERα mutation modifies the responsiveness of the cells to the growth factor signalling possibly through enhanced non-genomic activity of the mutant receptor, in certain cases of the invention.

Recent research into the mechanisms associated with tamoxifen (Tam) resistance indicate that some of the same growth factor receptor pathways implicated in adaptive hypersensitivity, such as Akt and MAPK, or specific oncogenes involved in intracellular signal transduction, become activated and are used to bypass normal hormonal responsiveness. Several reports indicate that the up-regulation of HER2 tyrosine kinase signalling in breast cancer plays an important role in the development of endocrine resistance (Arpino et al., 2008). Preclinical studies have demonstrated that HER2 overexpression in ERα positive MCF-7 human breast cancer xenografts rendered them resistant to Tam (Benz et al., 1992), and markedly increased levels of EGFR and HER2 were found in some sublines of MCF-7 cells with acquired Tam resistance (Nicholson et al., 2003; Hutcheson et al., 2003). Tam resistance in these cells was reversed by EGFR/HER2 tyrosine kinase inhibitors, and combined treatment with these inhibitors and Tam was effective in reversing resistance in xenograft models (Kurokawa et al., 2000), thereby strongly implicating this signalling network in resistance. Although HER2 overexpression occurs only in a minority of ERα-positive patients (Slamon et al., 1989), clinical studies confirm that HER2-overexpressing tumors are less responsive to Tam treatment (De Laurentiis et al., 2005; Osborne et al., 2003). In a previous retrospective study there was found an association between the K303R ERα mutation and elevated HER2 levels in invasive breast cancer (Herynk et al., 2007). Herein, HER2 up-regulation in K303R mutant-expressing cells was observed concomitant with an altered response to Tam with growth factor stimulation.

Mutant-expressing cells exhibit a higher level of growth under all conditions tested. Importantly, Tam sensitivity was significantly affected with estrogen and heregulin treatments. The data indicate that the Tam-resistant phenotype associated with the mutant was most pronounced in the presence of growth factor activation; in the presence of heregulin Tam inhibited soft agar growth only 21% compared with a 96% inhibition in WT ERα-expressing cells. In particular embodiments of the invention, bidirectional crosstalk between the HER2 and mutant receptors plays a role in conferring a selective advantage in terms of growth to those individuals that express K303R ERα mutant and are treated with Tam. The presence of the mutant in a retrospective cohort of patients treated with Tam who have long-term follow-up are investigated, in certain aspects of the invention.

Different therapeutic agents targeting the activity of the ErbB family of receptors have been recently developed and tested in patients. For instance, herceptin (Trastuzumab™), a monoclonal antibody against HER2, was approved for therapeutic use in patients with HER2-overexpressing breast cancer (Yeon et al., 2005; Tokunaga et al., 2006). By binding to the juxtamembrane domain of HER2 (Carter et al., 1992), this agent blocks HER2 homo- and heterodimerization with the other members of the ErbB family, and thereby interrupts the activation of downstream proliferative signalling. Herein it is shown that herceptin elicited its antiproliferative effect either under basal conditions or with hergulin treatment in K303R ERα mutant cells but did not affect growth of the WT cells. These findings confirm the embodiment that the HER2 pathway, which appears to be elevated in mutant cells, is involved in the regulation of cell growth in breast tumor cells bearing mutation, probably through increased crosstalk between HER2 and ERα pathways.

The existence of bidirectional crosstalk between ERα and growth factor receptor pathways, and its involvement in the development of endocrine resistance, has been well documented (Lee et al., 2001; Schiff et al., 2004). Several studies have demonstrated direct or indirect activation of growth factor signalling via ERα. For example, ligand-independent activation of serine 118 ERα by EGFR/MAPK-mediated phosphorylation regulates growth of tamoxifen-resistant MCF-7 breast cancer cells (Britton et al., 2006). Chung et al. have also demonstrated that HER2 and ERα can directly interact at the cell membrane (Chung et al., 2002), and this interaction protected breast cancer cells from Tam-induced apoptosis. Moreover, membrane or cytoplasmic ERα can induce phosphorylation of EGFR through activation of G-proteins, c-Src, and matrix metalloproteinases (Razandi et al., 2003), and can directly interact with adaptor proteins such as c-Src, Shc and the p85 cc regulatory subunit of PI3K (Migliaccio et al., 2005; Wong et al., 2002; Song et al., 2002; Sun et al., 2001). These processes activate downstream kinases that in turn activate ERα and its coregulatory proteins, thus also enhancing genomic activities of the receptor (Kato et al., 1995; Martin et al., 2000). All together these effects amplify the bidirectional crosstalk which multiplies signals between and downstream of the growth factor receptors and ERα, thus sustaining survival and proliferative signals in breast cancer cells.

The present findings indicate an enhanced hormone-independent physical association/complex between the mutant receptor and HER2 compared to WT receptor. This indicates that the mutation, present in the hinge region of the receptor, increases the ability of ERα to interact with HER2 or other components of the complex, in certain embodiments of the invention. As yet, the interaction surface between HER and ERα has not been defined. Altered interactions as were described indicates that Tam may not antagonize the mutant receptor because the HER2 pathway may be dominant and non-genomic action predominates, in certain aspects of the invention. This is consistent because it has already been demonstrated that the mutation can alter coregulator protein binding. The mutation demonstrates enhanced binding ability to bind to the TIF-2 coactivator (Fuqua et al., 2000), and the AIB coactivator at very low and physiological levels of estradiol, but decreased binding to the corepressor NCoR1. Mutant receptor binding to BRCA-1 has also been shown to be enhanced. These collective data indicate that altered affinity for ER coregulators, and possibly signalling molecules such as HER2, are a mechanism by which the K303R mutation confers hypersensitivity to low levels of estrogen, and reduced sensitivity to Tam, in particular aspects of the invention. The mutant receptor appears to exhibit increased ligand-independent activity that bypasses antiestrogen treatment.

To explore why the K303R ERα mutation has increased crosstalk with the HER2 pathway, the differences in post-translational modifications between WT and mutant receptor were examined. It is known that ERα activity of can be modulated by several post-translational modifications, such as protein phosphorylation (Likhite et al., 2006), acetylation (Kim et al., 2006), ubiquitination (Nawaz et al., 1999), sumoylation (Sentis et al., 2005), and methylation. The majority of studies in this field have focused their attention on the phosphorylation status of the receptor, and its effect on receptor activity. For instance, receptor phosphorylation by different kinases such as c-Src, PKA, MAPK, and Akt can all regulate receptor affinity, coregulator protein binding, and transcriptional activity (Likhite et al., 2006). It has been previously shown that the K303R ERα mutation renders the receptor a more efficient substrate for PKA-induced phosphorylation at residue S305 that has distinct biological results—enhanced hormone sensitivity for growth (Cui et al., 2004). Phosphorylation at the S305 residue can also be mediated by both protein kinase A (PKA) and p21-activated kinase-1 (PAK-1) signalling network (Cui et al., 2004; Rayala et al., 2006).

Several reports have identified the serine residue at 305 as a physiologically important site that modifies response to Tam. In particular it has been demonstrated using fluorescence resonance energy transfer (FRET) analysis that PKA signalling to ERα S305 causes a conformational arrest in the ERα and switches Tam from an antagonistic to an agonist (Michalides et al., 2004). Michalides et al. have also demonstrated that PKA phosphorylation at S305 ERα induces Tam resistance through an altered orientation of ERα towards the co-activator SRC-1 (Zwart et al., 2007). In addition, S305 ERαL phosphorylation by PAK-1 up-regulates cyclin D1 expression in breast cancer cells (Balasenthil et al., 2004). Herein it is shown that K303R mutant cells have elevated phosphorylation levels of S305 compared with WT-expressing cells indicating that the mutant have constitutive ligand-independent activity, in certain embodiments of the invention. The contribution of the S305 site in enhanced crosstalk with HER2 was established using a blocking Peptide. It was shown that heregulin-stimulation enhanced S305 phosphorylation in mutant expressing cells, but it did not significantly influence the phosphorylation status of WT receptor. Interestingly, the S305 peptide affected heregulin-induced ERα phosphorylation and prevented downstream phosphorylation events, such as activation of MAPK and Akt. These effects were even more prominent in K303R mutant cells, for example, due to prominent role of the S305 site in ligand-independent activity of the mutant receptor. A similar experimental approach was used by Varricchio et al. (2007). They demonstrated that a six amino acid peptide surrounding the phosphotyrosine residue 573 was able to block ER/c-Src interactions, cyclin D1 expression, and growth of MCF-7 and LNCaP cells.

It has been previously shown that transcriptional activity of the mutant receptor was induced at very low concentrations of estradiol (10⁻¹² M), and only the specific lysine to arginine substitution at 303 residue resulted in a receptor with enhanced sensitivity to estrogen. The inventors have also demonstrated that cAMP-dependent signalling can enhance the receptor's intrinsic sensitivity to hormone, and that blocking PKA activity reversed the hypersensitive proliferative phenotype in mutant-expressing cells (Cui et al., 2004). The data obtained herein using live cell dynamics agrees with these earlier results. The live cell high through-put analyses allowed study of mutant receptor/promoter interaction and chromatin remodelling (Sharp et al., 2006; Berno et al., 2006). The results confirm that the K303R mutant is inherently hypersensitivity to estrogen, and only WT receptor showed increased sensitivity to estrogen after forskolin treatment. The same experimental approach was utilized to test for altered Tam activity in the presence of forskolin. Tam treatment inhibited transcriptional responses in WT, but at statistically significant lower levels in mutant cells. Forskolin treatment blocked Tam-induced promoter condensation, indicating that ligand-independent kinase signalling to the mutant receptor decreases tamoxifen sensitivity.

Clinical studies have reported that breast tumors with HER2 amplification show reduced levels of progesterone receptor (Dowsett et al., 2001), and the absence of PR is a marker of a more aggressive phenotype (Balleine et al., 1999). Patients whose tumors lack PR derive less benefit from adjuvant hormonal therapy (Bardou et al., 2003; Ravdin et al., 1992; Elledge et al., 2000). In vitro studies indicate that amplified growth factor signalling may underlie the reduction of PR levels in breast cancer cells. (Cui et al., 2003). In this invention it was found that PR expression was almost undetectable in K303R cells under basal conditions, and estrogen induced only a small increase in PR content, compared to the effect elicited in WT expressing cells. The lack of PR expression observed in K303R mutant expressing cells may be the consequence of altered growth factor signalling that contributed to the Tam-resistant phenotype observed in the model system, in certain embodiments of the invention.

The K303R ERα hypersensitive phenotype involves an integration of post-translational modification events, such as phosphorylation at S305, with enhanced bidirectional crosstalk between the mutant and growth factor receptors such as HER2, and that genomic and nongenomic mechanisms contribute to Tam resistance, in certain aspects of the invention. Because the molecular and biological data demonstrate that the ERα mutation is resistant to Tam, for example, the mutation is a potential novel predictive marker of hormonal response in breast cancer tumors. In addition, the molecular studies indicate that use of a specific blocking peptide to prevent S305 phosphorylation of the mutant reduces ligand-independent activity and is a new therapeutic approach to treat individuals with mutation-positive tumors that are resistant to Tam therapy.

### Example 14

### Exemplary Methods for Detection of the Mutation

Exemplary Method 1

The mutation may be identified with the following exemplary method. Tumor DNA was isolated from a 2-μm-thick, 0.6-mm core tissue microarray using Qiagen DNeasy Tissue kits according to the manufacturer. The primer sequences used for PCR amplification and sequencing are shown in Table 1.

Table 1. Primer sequences for PCR amplification, dye-labeled terminator sequencing, and primer extension sequencing (SNaPshot)

| Primer | Use | Sequence (5′ to 3′) |
|---|---|---|
| ERα 1 | Forward PCR | ACATGAGAGCTGCCAACCTT |
| | | (SEQ ID NO: 15) |
| ERα2 | Reverse PCR | GGAATAGAGTATCGGGGGCT |
| | | (SEQ ID NO: 16) |
| ERα 3 | Forward | TTCATGATCAAACGCTCTAAGA |
| | extension | (SEQ ID NO: 17) |
| ERα 4 | Reverse | ACAAGGCCAGGCTGTTC |
| | extension | (SEQ ID NO: 18) |

For PCR amplification of ERα, an initial amplification using primers ERα 1 and 2 was done with a denaturation step at 95° C. for 10 min, followed by 35 cycles of denaturation at 95° C. for 1 min, primer annealing at 60° C. for 30 s, and primer extension at 72° C. for 30 s. Upon completion of the cycling steps, a final extension at 72° C. for 5 min was done before the reaction was stored at 4° C. To remove unincorporated PCR primers and deoxynucleotide triphosphates from the PCR amplification, the samples were treated by adding 2 units of exonuclease I (U.S. Biochemical), and 5 units of shrimp alkaline phosphatase (Roche Applied Science), for 1 h at 37° C. and 15 min at 80° C. These PCR products were used for both dye-labeled terminator and primer extension sequencing and may be used for mass spectroscopy and/or microarray SNP analyses, for example.

A negative control consisting of a PCR reaction without genomic DNA to ensure that no contaminating DNA was present, and a positive control of wild-type (WT) ERα genomic DNA from MCF-7 human breast cancer cells (previously determined to be WT sequence; Fuqua et al., 2000), were run in parallel with all tumor PCR reactions. Plasmids containing either WT or the mutant A908G ERα sequence (Fuqua et al., 2000) were used for PCR amplification in DNA mixing experiments to compare the dye-labeled terminator and primer extension sequencing methods.

Dye-labeled terminator automated fluorescent sequencing was done with an ABI PRISM BigDye Terminator cycle sequencing ready reaction kit with AmpliTaq DNA polymerase (Perkin-Elmer/Applied Bio systems Division) according to manufacturer's recommendations using either the ERα 1 primer (forward) or the ERα 2 primer (reverse). Primer extension sequencing was done using the ABI PRISM SNaPshot sequencing method (Perkin-Elmer/Applied Biosystems Division), which involves the extension of a primer that ends one nucleotide 5 of the ERα 908 nucleotide, using fluorescently labeled dideoxynucleotide triphosphates. The ERα 3 (forward) and the ERα 4 (reverse) primers were used for the extension SNaPshot reactions. All fluorescent sequencing products were analyzed on an ABI PRISM 310 Genetic Analyzer capillary sequencer (Perkin-Elmer/Applied Biosystems Division).

Data were analyzed with the ABI Gene Scan software package. Manual verification of sequencing results was also done. All tumor DNAs were first sequenced using SNaPshot in the reverse direction. To confirm this result, another aliquot of DNA from mutation-positive tumors was then PCR amplified again and resequenced using SNaPshot in the forward direction. Only those tumor samples with the A908G mutation detected on both the reverse and forward strand with SNaPshot sequencing were considered mutation positive.

Exemplary Method 2

Microdissection of specimens was performed on 55 samples using serial sections from formalin-fixed, paraffin-embedded tissue blocks as described (O′Connell et al., 1999). Briefly, alternative 3- and 10 um-thick sections were cut from the blocks and float mounted on glass slides. The 3-um-thick slides were stained with hematoxylin-eosin and examined under the light microscope to locate regions of normal and hyperplastic tissues; and these areas outlined with a felt-tipped pen. The marked slide was then used as a template to guide manual microdis section from the corresponding regions of the unstained 10-um-thick sections. It was possible to obtain distant normal tissue from 4 of the patients. A skilled artisan recognizes that there are a variety of methods to isolate desired cells from nondesired cells other than by manual manipulation or LCM. These include physical means of separating out undesired cells from desired cells, such as by centrifugation based on size, or centrifugation with magnetic beads attached to antibodies specific for desired and nondesired cell types.

DNA was liberated from the microdisseced specimens using a modification of the procedure of O′Connell et al (1999). Genomic sequencing was then performed using PCR amplification of isolated DNA using ER primer 1 (nucleotides 1093-1112 (5′ primer; 5′-CAAGCGCCAGAGAGATGATG-3′); SEQ ID NO:19) and ER primer 2 (nucleotides 1231-1250 (3′ primer); 5′-ACAAGGCACTGACCATCTGG-3′; SEQ ID NO:20) of the ER gene (Greene et al., 1996). An aliquot of this amplification was then used to perform single stranded PCR amplification using ER primer 3 (nucleotides 1221-1240 (3′ primer); 5′-GACCATCTGGTCGGCCGTCA-3′; SEQ ID NO:21) of the ER gene. After precipitation of the single stranded PCR amplification product, dideoxysequence analysis was performed using ER primer 4 (nucleotides 1099-1119 (5′ primer); CAGAGAGAATGATGGGGAGGG-3′; SEQ ID NO:4). In another embodiment, an alternative ER primer is used in lieu of ER primer 4, such as for nucleotides 1101-1130. Genomic DNA was isolated from normal blood samples of 80 healthy women, and utilized for genomic sequence analysis as described above. RNA was also isolated from four additional, frozen hyperplastic lesions, and utilized for RT/PCR amplification, cloning, and sequence analyses as described (Fuqua et al., 1991).

### Example 15

### Expression of the Mutation and AI Resistance

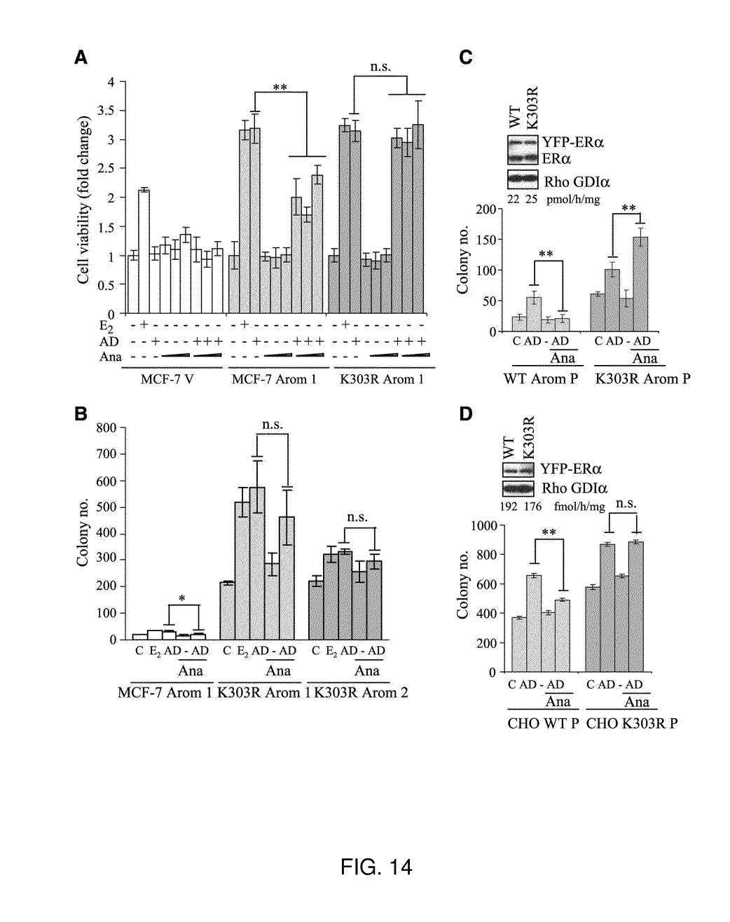

To investigate whether the mutant hypersensitive phenotype could cause AI^{R}, the inventor examined the effects of the nonsteroidal aromatase inhibitor Ana on cell growth (FIG. 14A). Growth of MCF-7 vector (V) cells was significantly enhanced by E₂, but as expected, they did not respond to AD treatment. AD treatment enhanced growth to the same extent as E₂ in MCF-7 Arom 1 cells, as well as K303R Arom 1-expressing cells. Ana treatment decreased AD-stimulated growth at ˜40% in MCF-7 Arom 1-expressing cells, but had no effect on the growth of mutant cells.

To extend the MTT data, anchorage-independent soft agar growth assays were performed (FIG. 14B). E₂ and AD treatments enhanced colony numbers, and treatment with Ana completely abrogated AD-stimulated growth of MCF-7 Arom 1 cells. In contrast, basal colony number was increased ˜10-fold in K303R Arom 1 and Arom 2 clones, and Ana was unable to inhibit AD-induced colony growth.

Because it was possible that overexpression of exogenous ERα alone might stimulate cell growth and contribute to the AI^{R} phenotype, the inventor also transfected MCF-7 Arom 1-expressing cells with an expression vector for YFP-WT ERα. Aromatase and exogenous YFP-ERα levels are shown (FIG. 14C, top). Pools expressing exogenous WT or mutant receptor were evaluated in soft agar assays (FIG. 14C, bottom). The basal growth of K303R ERα-Arom pools (P) was significantly higher than WT pools. Inhibition of aromatase activity by Ana caused a significant reduction in AD-stimulated growth in WT ERα Arom cells, but Ana was unable to reduce AD-stimulated mutant cell growth, confirming our stably transfected clones results. In the mutant pool AD +Ana stimulated growth compared with AD treatment alone, which may be a variable clonal effect. The inventor also generated stable pools of YFP-tagged ERα in ER-negative, aromatase-positive CHO cells (FIG. 14D). Ana did not reverse AD-stimulated growth in CHO mutant cells, confirming that the AI^{R} phenotype was associated with expression of the mutant in a number of different cellular backgrounds. It was also examined whether there was cross resistance to the nonsteroidal AI Exe. Exe treatment decreased AD-stimulated growth of mutant cells, although Exe was unable to reduce their high basal nonstimulated growth. Aromatase activity was effectively reduced by both Ana and Exe treatment.

### Example 16

### K303R ERα Aromatase Cells Exhibit Enhanced PI3K/Akt Signaling

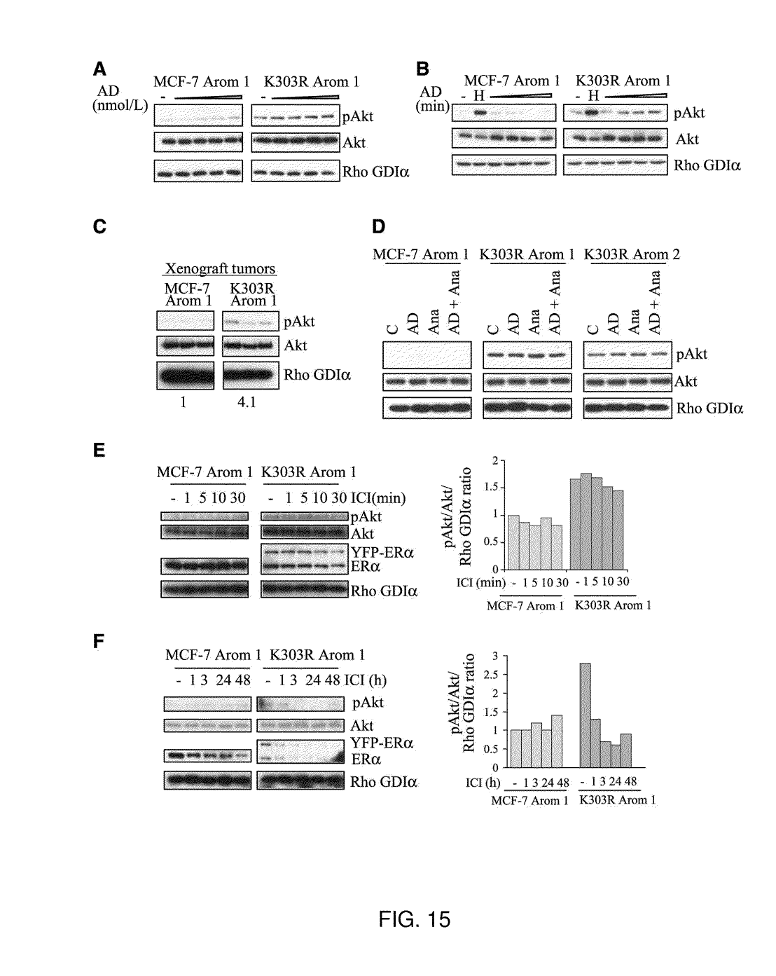

Previous studies have revealed the importance of increased MAPK activity and/or PI3K signaling in the adaptation of ERα-positive cells to long-term estrogen deprivation or AI treatment (Shim et al., 2000; Martin et al., 2003; Sabnis et al., 2005; Staka et al., 2005; Jelovac et al., 2005). To explore the mechanisms underlying the mutant AI^{R}, the inventor focused on short-term signaling events. Cells were treated with increasing AD concentrations (FIG. 15A) for different times (FIG. 15B). MCF-7 Arom 1 cells showed a low basal level of pAkt that was increased in a dose-dependent and time-dependent manner by AD treatment. In contrast, mutant cells showed elevated constitutive pAkt that was not further increased by AD; no changes in phosphorylated p42/44 ERK1,2/MAPK levels were seen. pAkt levels were also increased ˜4-fold in mutant tumor xenografts compared with MCF-7 Arom 1-overexpressing tumors (FIG. 15C).

The expression and activation of Akt in cells treated with Ana were next characterized (FIG. 15D). MCF-7 Arom-1 cells showed slightly increased levels of pAkt following AD treatment that was reduced with Ana cotreatment, whereas K303R Arom 1 and Arom 2-expressing cells showed elevated constitutive pAkt that was unaffected by Ana. It has been shown that treatment with the antiestrogen ICI182,780 leads to a rapid down-regulation in ERα levels (Pink and Jordan, 1996); there were no changes in pAkt with short-term treatment (FIG. 15E). Treatment with ICI182,780 (1-48 h) reduced ERα levels in MCF-7 Arom 1, but not basal pAkt; in contrast, ICI treatment resulted in a decrease in pAkt levels in mutant cells, concomitant with a reduction in YFP-ERα levels (FIG. 15F). This indicates that activation of pAkt is related to nongenomic activities of the mutant receptor, as short-term treatments did not block Akt phosphorylation, but are known to block ERα's genomic activities (Dauvois et al., 1993), in certain embodiments.

ERα can bind to the p85a regulatory subunit of PI3K and activate PI3K/Akt signaling in cells (Simoncini et al., 2000). It has been previously shown that the mutant ERα receptor exhibits altered binding with several nuclear receptor coregulatory proteins, such as the TIF-2 coactivator, and for the ERBB2 (HER2) oncogene when compared with WT ERα (Fuqua et al., 2000; Giordano et al., 2009). It was next examined whether the mutation might alter binding with the PI3K subunit p85, and alter PI3K activity. ER-negative CHO cells were transiently transfected with YFP-tagged receptors, and coimmunoprecipitation studies were performed.

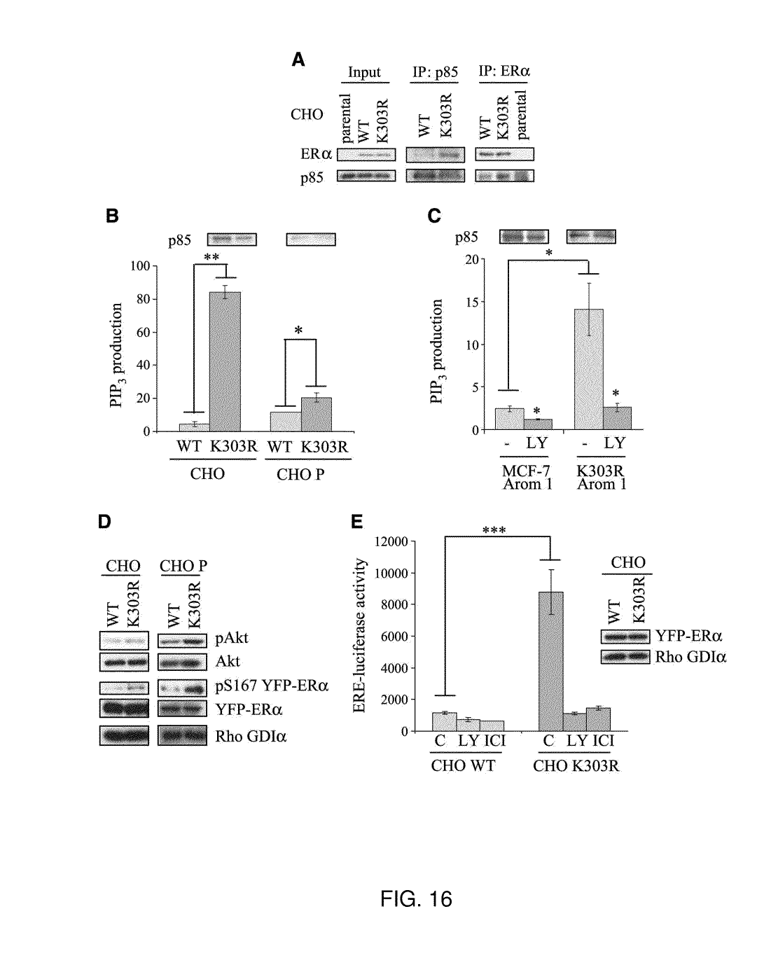

Enhanced binding of the p85 subunit to mutant receptor in the absence of ligand was observed (FIG. 16A). Equal amounts of lysates of CHO transfected cells (CHO+WT or K303R) or stable pools (CHO WT or K303R P; FIG. 16B) were immunoprecipitated with an anti-p85 antibody, and kinase activity was determined. Mutant ERα either transiently expressed or in stable pools significantly induced PI3K activity. The inventor also performed an in vitro PI3K assay in our stably transfected MCF-7 clones, and found constitutively increased PI3K activity in the mutant cells, which was abrogated by PI3K inhibitor LY294002 treatment (FIG. 16C). Elevated constitutive pAkt was detected in CHO cells transiently or stably expressing the mutant receptor (FIG. 16D). These data support a mechanism whereby enhanced binding of the regulatory subunit of PI3K results in enhanced downstream Akt signaling in the mutant cells.

Phosphorylation of ERα by a number of kinases is an important mechanism by which ERα activity can be regulated (Joel et al., 1998; Sun et al., 2001; Castano et al., 1997). Akt kinase phosphorylates ERα at serine (S) 167. Increased levels of pS167 YFP-ERα were found in K303R mutant cells (FIG. 16D), and the mutant increased ERE-transcriptional activity ˜8-fold (FIG. 16E). ERE activity was inhibited by treatment with either the PI3K inhibitor LY294002, or the antiestroogen ICI182,780. Thus, Akt may serve as a functional link between nongenomic and genomic ERα activities, engaging in a bidirectional cross-talk that may set up a vicious cycle between these two growth-regulatory pathways and augment signaling between them.

### Example 17

### K303R ERα-Aromatase Cells Exhibited Altered Apoptotic Responses

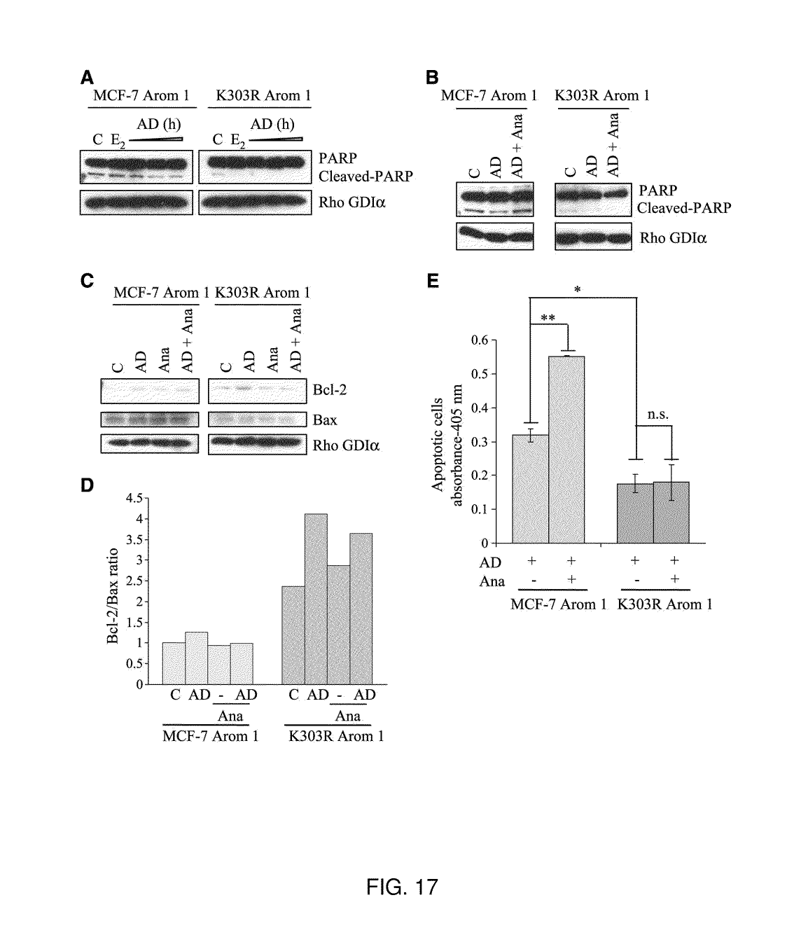

Akt/protein kinase B signaling is involved in the control of survival and apoptosis (Song et al., 2005; Kim et al., 2005), and AIs induce cell death by apoptosis (Thiantanawat et al., 2003). This suggestion led to question if the K303R ERα mutation could protect cells from apoptosis induced by Ana, providing these cells with a potential survival advantage. The inventor first evaluated PARP proteolysis, and found a reduction in the basal levels of the proteolytic form of PARP (85 kDa) in mutant cells under control conditions (FIG. 17A). AD treatment reduced its levels in a time-dependent fashion in WT and mutant cells, but this reduction was less pronounced in WT cells. E₂ was able to reduce PARP-cleavage levels only in mutant cells, suggesting that reduced apoptosis may underlie their hypersensitivity. Ana treatment of MCF-7 Arom 1-expressing cells increased proteolysis compared with AD treatment; cleavage was unchanged in mutant cells (FIG. 17B). In addition, there was an increase in the Bcl-2/Bax ratio in K303R Arom 1-expressing cells, which was further increased with AD and Ana treatments (FIGS. 17C and 17D).

To determine the levels of cellular apoptosis, the inventor also used ELISA cell death detection assays (FIG. 17E), and found that AD-stimulated mutant cells exhibited a lower apoptosis compared with MCF-7 Arom 1-expressing cells, indicating that the proliferative advantage provided by the mutation may be achieved by a decreased apoptotic response of these cells. In addition, Ana treatment induced an increase in cell apoptosis only in MCF-7 Arom 1 cells. In specific embodiments of the invention, mutant-expressing cells are resistant to AI-induced cellular apoptosis.

### Example 18

### The AI^{R} Phenotype is Dependent on the PI3K/Akt Pathway

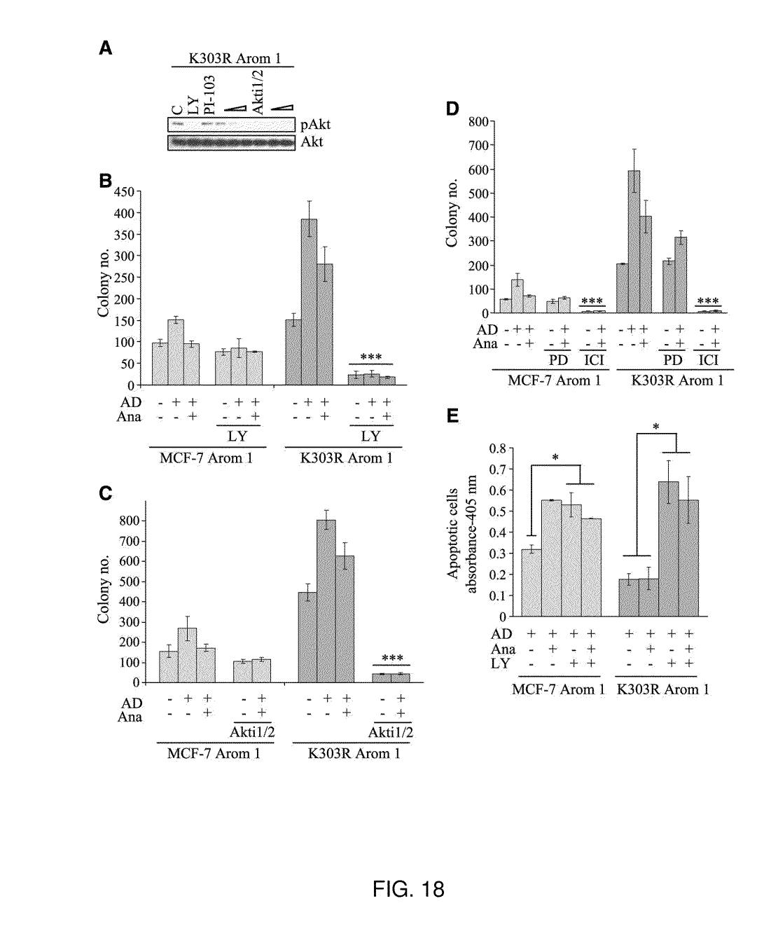

It was next addressed whether activated PI3K/Akt signaling may be a functional mechanism of resistance to AI therapies using PI3K/Akt inhibitors. To define the effective dose, mutant cells were treated with different doses of these agents and analyzed for pAkt. LY (10 μmol/L), PI-103 (1 μmol/L), Akti1/2 (1 μmol/L) effectively blocked basal Akt phosphorylation (FIG. 18A). Soft agar growth assays were then performed. LY completely inhibited mutant proliferation, whereas it induced a slight reduction in the growth of MCF-7 Arom 1 cells (FIG. 18B). Similar results were obtained using the PI-103 inhibitor in another mutant clone (K303R Arom 2). Because PI3K inhibitors affect not just all three Akt isozymes, but also other PH domain-containing molecules (DeFeo-Jones et al., 2005), the inventor tested a specific Akt inhibitor, Akti1/2 (FIG. 18C), and found that basal, AD-stimulated and Ana-stimulated growth was inhibited by ˜30% in MCF-7 Arom 1-expressing cells, whereas growth was inhibited by >90% in mutant cells. These results indicate that Akt signaling is essential for the growth of mutant cells, but may not play an important role in the growth of WT cells, in specific aspects of the invention.

A MEK1 inhibitor (PD98059) was also used in soft agar assays (FIG. 18D); antiproliferative effects were not observed and PD treatment was unable to reverse the AI^{R} mutant phenotype. These results confirm that the p44/42 Erk-1,2/MAPK pathway was not involved in the AI^{R} mutant phenotype. ICI182,780 (fulvestrant) was also used, and it suppressed the colony growth of both cell lines, indicating that ERα expression remained important in their growth regulation (FIG. 18D). The effects of the PI3K inhibitor LY294002 were evaluated on apoptosis using an ELISA cell death detection assay (FIG. 18E), and found that LY treatment in MCF-7 Arom 1 cells showed similar apoptosis induction as Ana-treated cells (1.7-fold increase), whereas inhibitor treatment of mutant cells was able to induce a 3.6-fold increase in the apoptotic rate compared with AD, and most significantly, after Ana treatment. The mutation provides a selective dependency on the PI3K/Akt survival pathway for sustained proliferation and/or survival. In specific cases of the invention, inhibition of this pathway addiction provides an effective means to reverse AI^{R} associated with the expression of K303R ERα mutation.

### Example 19

### Exemplary Materials and Methods for Examples 15-18

Reagents, hormones, and antibodies. 17β-Estradiol, 4-androstene-3,17-dione and heregulin were obtained from Sigma. Anastrozole and ICI182,780 were provided by AstraZeneca. PD98059, PI-103, Akt inhibitor VIII isozyme-selective (Akti1/2), and LY294002 were from Calbiochem. Exemestane (Exe) was obtained from Pfizer. Antibodies used for immunoblotting were ERα (clone 6F11) from Vector Laboratories and Rho GDIα from Santa Cruz Biotechnology. Total ERK1,2/MAPK, total Akt, phosphorylated p42/44 ERK1,2/MAPK (Thr²⁰²/Tyr²⁰⁴), Akt (Ser⁴⁷³), ERα (Ser¹⁶⁷), and poly(ADP ribose)polymerase (PARP) were from Cell Signaling Technology; Bax and Bcl-2 were from Calbiochem; cytochrome P450 aromatase was from Serotec; and p85 was from Upstate Biotechnology. Secondary antibodies goat anti-mouse or goat anti-rabbit were obtained from Amersham Biosciences.

Plasmids. Full-length human aromatase cDNA was amplified from the pCMV6-Arom plasmid (OriGene Technologies) by PCR using the following primers: forward 5¶-ACACTAGTATGGTTTTGGAAATGCTGAACCC-3 (SEQ ID NO:22) and reverse 5¶-ACGCGGCCGCCTAGTGTTCCAGACACCTGTCT (SEQ ID NO:23). This PCR product was subcloned into the Spe I/Not I sites of the pZeoSV2-vector (Invitrogen). The resulting pZeoSV2-aromatase expression vector (pZeo-Arom) sequence was confirmed by DNA sequencing. Generation of yellow fluorescent protein (YFP)-tagged expression constructs, YFP-WT and K303R-ERα, has been previously described (Cui et al., 2004).

Cell culture. MCF-7 parental breast cancer cells were cultured as described (Cui et al., 2004). MCF-7 wild-type (WT) and K303R ERα-expressing cells were generated as described (Herynk et al., 2006). MCF-7 parental and YFP-K303R ERa clones were stably transfected with the pZeo-Arom expression vector using Fugene 6 reagent according to the manufacturer (Roche). Chinese hamster ovary (CHO) or MCF-7 Arom-expressing pools, stably transfected with YFP-WT ERα and YFP-K303R ERα expression vectors, were also used.

Aromatase activity assay. Aromatase activity was evaluated using a ³H-water release assay using 0.5 μmol/L of [1β-³H]-androst-4-ene-3,17-dione as substrate (Lephart and Simpson, 1991). The incubations were performed at 37° C. for 1 h. The results were expressed as fmol-pmol/h/mg protein.

Tumor xenografts. All animal studies were carried out according to the guidelines and with the approval of the Baylor College of Medicine Animal Care and Use Committee. Female nude ovariectomized athymic mice were injected with MCF-7 YFP-WT and YFP-K303R ERα-expressing cells or MCF-7 Arom 1 and K303R Arom 1-expressing cells as described (Osborne et al., 1995). Animals were supplemented with estrogen tubing releasing ˜80 pg/mL, or supplemented daily with injections of the aromatase substrate androstenedione (AD; 100 μg).

Cell proliferation assays. Seven hundred cells were plated into 96-well plates in phenol red-free MEM containing 5% charcoal-stripped fetal bovine serum (FBS). After 24 h, cells were treated as indicated. Nine days later, proliferation was assessed using 3-(4,5-dimethylthiazol-2-yl)-2,5-diphenyltetrazolium bromide (MTT) reagent following the recommendations of the manufacturer. Soft agar anchorage-independent growth assays were performed as previously described; data shown are the mean colony numbers of three plates and is representative of two or three independent experiments (Giordano et al., 2009).

Immunoprecipitation and immunoblot analyses. Cells were starved in serum-free MEM for 48 h and treated as indicated before lysis (Herynk et al., 2007). For coimmunoprecipitation experiments, the inventor used 500 Ag of total cellular protein and anti-ERa (1:50) or anti-p85 (1 μg) antisera overnight and protein A/G with rotation at 4jC for 2 h. Protein extracts from tumor tissues were prepared by homogenizing the tissue in lysis buffer supplemented with 10% glycerol. Equal amounts of extracts were subjected to SDS-PAGE as described (Cui et al., 2004). Blots shown are representative of two or three individual experiments.

PI3K activity. PI3K was immunoprecipitated from cellular lysates with p85 antibody and the amount of phosphatidylinositol-(3,4,5)-trisphosphate (PIP₃) produced was assessed in a competitive ELISA (Echelon Biosciences).

ERα transactivation assays. Cells (50,000/well) were plated in phenol red free MEM with 5% charcoal-stripped FBS in 24-well plates, and then cotransfected with 0.5 μg of an ERE₂-tk-luciferase reporter plasmid, plus 1 ng of the respective ERa expression plasmid and 50 ng of CMV-h-galactosidase plasmid as an internal control using Fugene 6 reagent. After 6 h, cells were treated as indicated for 18 to 24 h. Luciferase activities were determined as described (Cui et al., 2004).

Apoptosis assays. Cells were seeded into six-well plates (10⁵/well) in phenol red-free MEM with 5% charcoal-stripped FBS for 48 h and then incubated with different treatments for 72 h. Apoptosis was measured using the Cell Death Detection ELISA Kit (Roche). Data are representative of two independent experiments performed in triplicate.

Statistical analyses. Data were analyzed by Student's t test using the GraphPad Prism5 software program.

### Example 20

### Development of Clinical Diagnostic Assays and Targeted Therapies for the Hypersensitive K303R ERα Mutation in Breast Cancer

### Overview

The inventor identified that the K303R ERα mutation in hormone-refractory breast cancer confers resistance to the aromatase inhibitor (AI) anastrozole through constitutive activation of the PI3K/AKT pro-survival signaling pathway to which the mutant cells have become “addicted” for maintenance of growth. As described herein, expression of the K303R ERα mutation can block tamoxifen's antagonist actions when engaged in cross-talk with growth factor receptor signaling pathways, rendering tumors hormone-refractory. These data indicate that in certain embodiments human breast cancer cells expressing the K303R ERα mutation escape from growth inhibition when treated with either of the two main classes of hormonal agents (AIs and tamoxifen) used in breast cancer patients today, so genetic assays designed to selectively and sensitively detect the mutation offer new predictive markers for hormonal response in breast tumors, in particular aspects of the invention.

In certain embodiments, the invention relates to ERα-positive breast cancer, the most common type. Of the approximate 180,000 women diagnosed per year in the US, 75% have ER-positive tumors, of which about 90% are negative for the growth factor receptor HER2, so hormonal agents are their only targeted option. It is known that while recurrences can be meaningfully delayed for years with hormonal agents, the clinical challenge is that resistance ultimately occurs. Compounding the problem, once resistance to sequential hormonal therapies occurs, chemotherapy active in ER-negative tumors, which could be offered to women with hormone-resistant disease, may not be as beneficial. In one embodiment of the invention, there are robust diagnostic assays for detection of the mutation. In another embodiment of the invention, there are clinically useful assays for the resistance pathways associated with the mutation in order to develop and then implement strategies to convert hormone-resistant tumors back to being sensitive via targeted biologic therapies. In certain aspects of the invention, the ERα S305 phosphorylation site adjacent to the K303R mutation is a target to block ERα hyperactivation and resistance. A reversion to hormone sensitivity of mutation-positive patients has massive clinical impact, allowing women to remain on hormonal agents, which in general preserve a good quality of life for many years.

### Optimization of DNA Mutation Detection in Hormone-Refractory Breast Cancers

Because the presence of the K303R ERα mutation (A908G transition) in breast tumors had been controversial, the inventor first set out to compare the genomic DNA sequencing method used in the literature (dye-labeled terminator automated fluorescent sequencing) with an optimized method (primer-extension SNaPshot™ sequencing). Using mixing experiments of PCR-amplified DNA from either wild-type (WT) or mutant-containing plasmids, both WT and mutant sequences were correctly genotyped using the primer-extension method (Herynk et al., 2007). However in diluted mixes (25% and 43% mutant-containing DNA mixes), dye-labeled terminator sequencing called the samples homozygous WT, and failed to detect the mutation. It has been demonstrated that there are reproducible peak height patterns using the dye-labeled terminator sequencing method, and that specific three or four-base pair combinations can affect base pair heights of the 3′ base. For instance, the three-base combination GAA results in a small peak height for the 3′ base (A), and this three-base combination is the same sequence found in the ERα 908 WT forward direction. Similarly, sequence-dependent incorporation may be complicating the discrimination of the mutant C base on the reverse strand as well—the sequence TTCC is problematic in that the 3′ C peak can be smaller using dye-terminator chemistry, and this is the same sequence preceding the mutation in the ERα reverse strand. Thus, the identification of heterozygote mutant individuals via dye-labeled terminator sequencing method may be difficult because of uneven peak heights, especially in heterogeneous tumor samples where contaminating normal cells further dilute the mutant signal as was reported by one group of investigators (Herynk et al., 2007).

However, an optimized primer-extension SNaPshot™ method can be laborious. This method is employed to characterize the mutation as a predictive marker of hormone-refractory disease, using n=286 invasive breast cancers maintained in an archived tumor bank. The patients in this study were derived from a prospectively assembled tumor bank. Tumor samples were archived in the form of formalin-fixed, paraffin-embedded medium density tissue microarrays as described (Ma et al., 2006). Patients were diagnosed between 1973 and 1993 with primary breast cancer, treated with mastectomy or lumpectomy plus axillary dissection, with or without post-operative radiation therapy, and all of the women underwent adjuvant Tam monotherapy. The median follow-up of living cases is about 7 years.

There are a number of high-throughput genotyping technologies currently available that are efficient. A high-throughput method for detection of the ERα mutation in DNA is developed using, for example, matrix-assisted laser desorption/ionization time-of-flight mass spectrometry (MALDI-TOF MS). This method (called Sequenom MassArray™) can detect a mutation even if it is present in only 5% of the cell population, which makes it ideal for detecting mutations found in a mixture of tumor and normal tissue. The assay also requires very little DNA, and can utilize degraded DNA like that obtained from formalin-fixed, paraffin-embedded tissues (Jaremko et al., 2005). The method also uses primer-extension chemistry, which we have found thus far to be optimum for the sequence surrounding the mutation. One of the key advantages to using the Sequenom method is that it does not use dye terminator chemistry and thus we should avoid some of the difficulties that are known to occur in TTCC sequences. Primers are optimized using WT and mutant ERα plasmids, as well as genomic DNA from MCF-7 cells stably transfected with these plasmids. Sequenom has recently developed proprietary chemistry to overcome GC compression problems, which should also prove beneficial for the sequence surrounding the K303R ERα mutation. Thus, in some embodiments one can use the Sequenom MassArray™ technique, and compare the results with those obtained using the SNaPshot™ method. One can sequence both strands, and discordances can be repeated and scored together for statistical comparisons of the presence of the mutation with outcomes. Only those tumor samples with the mutation detected on both the reverse and forward strands with both methods are considered mutation-positive, in certain cases.

The inventor has developed a standard approach to analyzing predictive clinical studies, especially those that use valuable tumor bank material. An empirically developed method, which the inventor has used since 1994, has recently been confirmed theoretically (Schmoor et al., 2000). In one embodiment, the proportion of cases with the mutation is at least 33% (the rate in previous studies of similar cases from the same bank using untreated cases was about 50%), and association between mutation status and other important prognostic variables, such as nodal status, is equivalent to a correlation of |r|=0.5 or less. This latter estimate is also based on previous data from untreated early breast cancer cases (Herynk et al., 2007). The censoring rate (i.e. non-relapsed) in this bank is about 70% for time to relapse (TTR), and about 60% for overall survival (OS). About a third of deaths in this cohort are due to causes other than cancer, and one can therefore focus on TTR as the outcome most reflective of cancer biology, although OS is also examined. With this sample size (N˜286) and rate of censoring, one can expect to detect hazard ratios of 2.0 and 1.8, respectively, for TTR and OS, at the alpha=0.05 level with 80% power. One can expect to see a 5 year TTR of 75% in all cases combined, and an HR of 2.0 is equivalent to a 5 year TTR of 82% in the WT group, compared to 67% in the mutant group. One can have 90% power to detect HR's of 2.2 and 2.0 respectively. Analyses of these studies can proceed in a stepwise manner with summarization of cases, estimation of associations between mutation status and other previously assessed clinical and biological characteristics, and finally, survival analyses using both univariate and multiple variable methods such as Cox proportional hazards regression.

### Expression of the K303R ERα Mutation Confers Hormone Resistance

One can focus on optimizing the mutation detection method using samples first from control samples (plasmids and stably transfected cell line models), and then from the clinical study of tamoxifen-treated patients described above. The rationale for this is recent data using ER-positive MCF-7 cell line models expressing the mutation. The K303R ERα mutation conferred resistance to tamoxifen treatment when cells were also exposed to the growth factor heregulin, indicating that the mutation participates in bidirectional cross-talk with HER2 to evade tamoxifen antagonist activity (Giordano et al., 2009). The mutation also confers resistance to the aromatase inhibitor anastrozole in MCF-7 cells also co-expressing human aromatase. The inventor evaluated growth in anchorage-independent assays using the aromatase substrate androstenedione (AD) with or without the AI anastrozole (Ana) in MCF-7 cells expressing WT ERα (MCF-7 Arom 1), and two MCF-7 clones expressing the K303R ERα mutation (see FIG. 2C). Estrogen and AD treatments enhanced colony numbers, and treatment with Ana completely abrogated AD-stimulated growth of MCF-7 Arom 1 cells with WT ERα. In contrast, basal colony number was increased ˜10-fold in K303R Arom 1 and 2 clones, and Ana was unable to inhibit AD-induced colony growth. The inventor then investigated the expression and activation of Akt in cells treated with Ana (see FIG. 15D). MCF-7 Arom-1 cells showed slightly increased levels of pAkt following AD treatment that was reduced with Ana co-treatment, while K303R Arom 1 and 2-expressing cells showed elevated constitutive pAkt that was unaffected by Ana. The inventor has also determined that the AI-resistant phenotype associated with mutant expression was dependent on the PI3K/Akt pathway. This is shown in the soft agar growth assay in FIG. 3B.

The inventor tested a specific Akt inhibitor, Akti1/2 (Calbiochem), and found that basal, AD-, and Ana-stimulated growth was inhibited by ˜30% in MCF-7 Arom 1-expressing cells, while growth was inhibited by >90% in mutant cells. These results indicate that Akt signaling is essential for the growth of mutant cells, in certain aspects of the invention. Based on this data, in one embodiment of the invention the mutant may be “addicted” to this survival pathway. Therefore in some embodiments a multiplexed Sequenom MassArray™ is developed to examine the K303R ERα mutation in conjunction with mutations associated with activation of the PI3K and Akt pathways. There are already available Sequenom Snp assays for Akt and PI3K (AKT1_E17K, PIK3CA_A1046V, PIK3CA_C420R, PIK3CA_E110K, PIK3CA_E418K, PIK3CA_E453K, PIK3CA_E542K, and PIK3CA_E542V).

In certain embodiments of the invention there is the following: A) optimization of multiplexed Sequenom MassArray™ assays for the mutation and the Snps listed above, B) sequencing of the clinical samples (tamoxifen-treated cohort) by SNaPshot™, C) sequencing of these same clinical samples with the optimized multiplex assay, D) comparison of the two sequencing methods and reanalysis of discordant samples, E) statistical analysis of associations of the mutation with outcome. Thus, one can employ at least one of these exemplary methods to determine whether the mutation predicts a poorer outcome in tamoxifen-treated breast cancer.

If the Sequenom method is not sufficiently sensitive, one can utilize other technologies, such as Fluidigm® BioMark™ and BioTrove OpenArray® that use existing Taqman DNA or RNA material, but are more sensitive and require less starting material, or Transgenomics Wave that is a high pressure liquid chromatography-based method.

RNA-Based Assays of ERα Expression—could Current Immunohistochemical (IHC) Assays be Replaced?

With the development of specific, reliable, and commercially-available ERα antibodies, and the growth of robust IHC technologies, IHC assays have replaced ligand-binding assays to measure ERα levels (Harris et al., 2007). IHC allows for the precise determination of ERα status at the individual cell level, accommodating the problem of tissue heterogeneity within the tumor. Importantly, IHC can be performed on retrospectively collected, formalin-fixed paraffin-embedded tissue, including archival tissues. Thus, IHC assays of ERα have become the standard method to determine whether a patient is a candidate for hormonal therapy. But because IHC scores of receptor status using highly sensitive methodology can be dichotomous (Collins et al., 2005), accurate quantization may be better suited for RNA-based assays, such as quantitative reverse transcription-polymerase chain reaction (qRT-PCR). The development of the robust 21 gene Oncotype DXTM qRT-PCR clinical predictive assay using fixed tissue material is evidence that it is possible to reliably measure ERα RNA levels from archived tissues (Paik et al., 2004), and many breast cancer clinicians are currently using this predictive assay in their practice. Badve et al. have compared central laboratory IHC vs. qRT-PCR assays for measuring ERα levels in samples from ECOG 2197 (Badve et al., 2008). In these samples, measuring ERα by qRT-PCR was statistically superior to IHC in predicting relapse in tamoxifen-treated, ERα-positive patients. In conclusion, qRT-PCR is an alternative method for determining hormone receptor status and may circumvent the limitations of non-standardized IHC assays. Therefore, in specific embodiments a number of different qRT-PCR assays are utilized to measure the K303R ERα mutation in RNA, in certain aspects of the invention. In specific embodiments the mutation is reliably measured starting with RNA, but studies are needed to determine if qRT-PCR assays as proposed herein could indeed outperform DNA-based assays for validation of the mutation's association with poor patient outcome.

In one embodiment, material from an RNA bank maintained in the inventor's laboratory from 400 frozen invasive breast tumors collected during the 1980's (n=200 ER-positive, and 200 ER-negative tumors; ER status was determined by ligand-binding assay) is employed. None of these RNAs have associated clinical information other than ER status, so they are optimum for assay development. These RNAs are used to determine the frequency of the mutation in invasive breast tumors using RNA-based assays that could be suitable for routine diagnostic laboratories. Again, using the inventor's DNA-based SNaPshot™ assay, they have previously determined that the mutation was present in ˜50% of invasive tumors (Herynk et al., 2007), and a similar frequency is obtained using RNA-based methods, in specific cases. One can make cDNA using ER-specific reverse transcription from these 400 RNA samples (Fuqua et al., 1990), and sequence the cDNAs for the mutation, such as via Real-Time TaqMan® PCR (Applied Biosystems) coupled with hybridization probes with melting curve analyses, hydrolysis probes, molecular beacons, and scorpion primers, for example. In some embodiments, as important mutations/Snps like the K303R ERα mutation are discovered, assays such as the Oncotype DX™ qRT-PCR predictive assay are incorporated into mutation analysis.

The K303R ERα Mutation is Associated with Clinical Measures of Poor Outcome in Untreated Patients

As mentioned above, the inventor performed SNaPshot™ sequencing on a cohort of 267 primary breast tumors from untreated patients with known clinical outcomes with the goal of examining the role of the mutation in the natural history of these patients (Herynk et al., 2007). The mutation was significantly more frequent in women >50 years of age, consistent with the embodiment that the mutation plays a role in postmenopausal women with breast cancer. The mutation was also more frequent in node-positive tumors, and was significantly associated with larger tumor size. Both axillary lymph node positivity and larger tumor size are established clinical variables associated with a poorer outcome. However the mutation was not an independent predictor of outcomes in multivariate analyses (though in univariate survival analyses it did predict a poor outcome), probably because of its strong relationship with nodal status and tumor size.

In certain cases of the invention, one can use an optimum RNA-based sequencing method in another RNA bank prepared from ˜100 invasive breast tumors from untreated patients with long-term clinical follow-up (median 7-8 years). It is useful to characterize the significance of the mutation in another independent cohort with associated clinical outcome and biomarker data. These tumor samples were obtained from Asterand, and were maintained frozen until RNA was extracted, so that the RNA is of excellent quality. Clinical characteristics and the presence of the mutation are evaluated using Spearman's rank correlations. Univariate and multivariate analysis are performed.

### ERα Post-Translational Modifications and the Mutation

The inventor and others have discovered that extensive post-translational modifications—sumoylation, acetylation, methylation, and phosphorylation—can all occur on residues surrounding the mutation site within the hinge domain (Cui et al., 2005; Wang et al., 2001). In one embodiment, one of the reasons mutation of this site may be selected for during tumorigenesis is because it is such an important regulatory region. The same ERα lysine residues that are acetylated, including K302 and K303, can also be sumoylated, suggesting a tight regulation between phosphorylation events at S305 with the processes of ubiquitination, acetylation, and sumoylation. A recent report has shown that the K303R ERα mutation can alter methylation at K302 (Subramanian et al., 2008). In some embodiments, acetylation of K303 attenuates ER-driven transcription, not just from antagonism via acetylation, but also by inhibition of K302 methylation and subsequent destabilization of ERα.

Further support for the importance of this region of steroid receptors, also called the carboxyl terminal extension (CTE) of the DNA binding domain, is work showing that it is a multifunctional domain that binds co-regulatory proteins and participates in binding of receptors to DNA. The CTEs of the progesterone and androgen receptors also undergo post-translational modifications by phosphorylation and acetylation. Furthermore, naturally-occurring mutations have also been identified in the CTE of the progesterone receptor in ovarian cancer, and the androgen receptor in prostate cancer (Agoulnik et al., 2004; Pijnenborg et al., 2005). These mutations affect acetylation or phosphorylation of the CTE and enhance sensitivity to hormone, similar to what the inventor has reported for the A908G ERα mutation.

Phosphorylation at serine (S) 305 in ERα is another particularly important site. The K303R mutation makes this site a better substrate for ligand-independent phosphorylation by PKA and p21-activated kinase 1 (PAK-1) pathways, which then causes a cell to become hypersensitive to estrogen and less responsive to tamoxifen treatment (Cui et al., 2004; Michalides et al., 2004). These data demonstrate that these coordinated and dynamic post-translational events have important effects on hormone response; therefore the development of reagents to them could prove to be clinically as well as scientifically useful. The data indicate that S305 phosphorylation plays a key role in AI resistance, and enhanced downstream signaling (FIG. 9B, right panel). Anchorage-independent growth assays in soft agar were performed on MCF-7 pools stably expressing aromatase (WT Arom P), or aromatase plus the K303R mutant (K303R Arom P), or aromatase plus a double mutation (K303R/S305A) to destroy the S305 phosphorylation site.

Cells expressing the K303R mutation exhibited enhanced colony growth in the presence of AD, highlighting the estrogen-hypersensitive phenotype in these cells; anastrozole (Ana) treatment further stimulated the growth of these cells, demonstrating that these cells are resistant to AI treatment. Mutation of the S305 phosphorylation site blocked growth and restored AI sensitivity. These results indicate that the S305 site may be a target to restore AI sensitivity, and this is further characterized.

The inventor next performed immunoblot analysis to evaluate the phosphorylation levels of Akt after incubation with an S305 blocking peptide (FIG. 9B). To block phosphorylation at S305 the inventor delivered a short peptide competitor (S305 peptide, ERα residues 298 to 310) to the cells. After peptide delivery, the cells were subjected to short term treatment (10 min) with heregulin and then analyzed by immunoblot analysis. Heregulin enhanced S305 phosphorylation in K303R mutant-overexpressing cells, but had no effect on phosphorylation of this region in WT MCF-7 cells. Phosphorylation of the mutant receptor was abrogated by the S305 peptide. Addition of the S305 blocking peptide also inhibited heregulin-induced phosphorylation of Akt in both cell lines. These data indicate that phosphorylation of the S305 residue is useful in mediating enhanced cross-talk between downstream signaling pathways and the mutant receptor, and indicate that phosphorylation antagonism is a therapeutic strategy to block mutant function, in certain aspects of the invention.

There are commercial antibodies available to S305 (an antibody from Bethyl Labs, for example). However, one can produce monoclonal antibodies to methylated K302/303, acetylated K302/303, and phosphorylated S305 synthetic peptides. The specificity of the antibodies is evaluated using immunoblot, immunohistochemistry, and immunoprecipitation analysis with our MCF-7 cell line models, and the appropriate recombinant protein controls (for instance K302R, K303R, S305D, and S305A-GST-tagged proteins). Thus, reagents are developed that can be used in archived clinical samples to determine if post-translational modifications predict hormone resistance. A recent IHC study suggested that ERα S305 phosphorylation status could indeed predict resistance to tamoxifen in patients (Holm et al., 2009).

Development of Monoclonal Antibodies (MAbs) Directed Against Post-Translational Modifications in the Hinge Region of ERα Surrounding the Mutation, and Evaluation of their Utility in Detection of these Markers in Breast Tumors

The generation of monoclonal antibodies may include immunization of Balb/c mice, ELISA screening of mouse sera, removal of spleen cells and fusion with mouse myelomas, growth selection of hybridomas, ELISA screening and subcloning of positive hybridomas.

Peptide conjugates are used as immunogens to generate MAbs to specific phosphorylation, methylation and acetylation sites of ERα. The following exemplary peptides, corresponding to sequences in the hinge region of human ERα, are synthesized by Pi Proteomics, LLC (Huntsville, Ala.) with modification groups including a single phosphate at serine 305, dimethylation of lysine 302, 303 or diacetylation of lysine 302, 303. The peptides will be conjugated to keyhole limpet hemocyanin (KLH) through multiple chemistries including gluteraldehyde for N-terminal NH2 groups, EDC for C-term and OH groups and maleimide for SH groups. The conjugations achieve a 1:1 mass ratio of peptide to KLH carrier and results in multiple orientations of the coupled peptide to increase its antigenicity. As an alternative to enhance antigenicity, peptides can also be synthesized with two Gly-Gly spacers and a C-terminal Cys for directed sulfhydryl conjugation. Peptides will be synthesized with the following formats either with or without glycine spacers: (ERα aa 297-308): NH2-MIKRS(₅)Ac(K)Ac(K)NSLAL(₅)-COOH-acetylation K302/303 (SEQ ID NO:25); (ERαL aa 297-308): NH2-MIKRS(₅)Me(K)Me(K)NSLAL(₅)-COOH-methylation K302/303 (SEQ ID NO:26); (ERαL aa 299-310): NH2-KRSKKN(₆)p(S)LALSL(₅)-COOH-phosphorylation S305 (SEQ ID NO:27). For each chemical modification, five different peptides, for example, are made: (a) free unmodified peptide (screening) (b) free modified peptide (screening) (c) KLH conjugated modified peptide (immunization only) (d) BSA conjugated modified peptide (screening) and (e) BSA conjugate unmodified peptide (screening).

Antibodies are produced in mice. Five BALB/cJ mice are injected with 100 μg of each peptide conjugate in Freund's complete adjuvant. Mouse sera will be screened after the last boost using both an ELISA against peptide antigen coated plates (BSA-conjugates) and a western immunoblot against extracts from cells transfected with wt ERα or ERα with point mutations, S305A, K302A, or K303A. A secondary screen will be performed by Western immunoblotting using MCF-7 cells transfected with WT ERα or ERα in which the targeted modification-sites have been mutated to alanines to assure that the MAbs detect the specific modification site in the context of intact full-length receptor. Extracts from MCF-7 cells expressing endogenous ERα and the mutant are analyzed by immunoblotting to select the most sensitive MAbs. A final screen is performed using immunocytochemistry. Again the MCF-7 cell line models transfected with WT ERα or the mutant ERα with point mutations in the modified sites, or cells in which endogenous ERα has been knocked down using a specific shRNA, are formalin fixed and sections prepared and screened for specific MAb staining by immunoperoxidase detection methods. Those hybridomas found to produce antibodies with specificity for chemically modified sites at K302, 303, or S305 by the criteria above re cloned by limiting dilution, and used for large-scale production of antibodies.

In one embodiment, antibodies that can be used by immunoblotting or IHC are developed to examine ERα post-translation modification status in breast tumor biopsies. Therefore as an alternative, in addition to the traditional screening procedures outlined above, one can employ high throughput fluorescence microscopy (HTM) as a primary screen for identification of MAbs with the desired properties i.e. those that will work in formalin fixed sections. For this approach, cells (Hela) are prepared that express WT ERα or ERα bearing specific mutations in the targeted modification sites, plated into 96-wells and fixed with 3.7% formalin. In parallel with ELISA assays, the wells will be incubated with supernatant from the hybridomas (typically ˜2,000 hybridomas are screened) and screened using HTM. Positive cells will be determined by specific nuclear staining that is preferential for cells expressing wt ERα as compared with mutant ERα. Clones that are preferentially positive for modified peptides by ELISA and by HTM will be subcloned and the specificity of the secreted MAb are confirmed using the standard approaches outlined above. HTM is a novel approach for primary screening of hybridomas that should enable the selection of MAbs a priori that work well by IHC. The more traditional approach of screening hybridomas is by ELISA and Western immunoblotting and to then hope that the final MAbs selected have the desired properties to detect the antigen by IHC.

IHC assays with the antibodies are developed. The initial testing identifies the best antigen retrieval condition in formalin-fixed paraffin-embedded tissue sections. This is achieved by treating the tissue sections with a variety of buffer or enzyme solutions. The enzyme systems include trypsin, pepsin, pronase, proteinase K, etc., and in general are less predictable in their efficiency as compared to the buffer systems. Unless otherwise indicated, tissue sections are treated in a pressure cooker containing AR buffer solutions at near-boiling temperatures. The next level of testing is to establish optimum sensitivity of the assay. Ideally, if the expression of the protein is heterogeneous, then the optimized assay should be able to detect all levels (low, intermediate, and high) of protein expression in a given tissue type. Adjusting the concentration of the primary antibody, time of incubation of the primary antibody, and sometimes concentration of the detection system components attains this objective.

### Peptide Antagonist to ER

The data shown in FIG. 9B indicates blocking phosphorylation of S305 and its downstream signaling effects is a useful therapeutic strategy. Therefore, one can prepare peptide mimetics (different regions of ERα encompassing these post-translational modifications) and use them in growth assays, or one can have the peptides acetylated, methylated, or phosphorylated in vitro, and then test for their effects on cross-talk using immunoblot analysis, and on growth in anchorage-independent assays. These studies determine if the effects one sees on S305 phosphorylation and signaling ultimately targets the hypersensitive and antiestrogen-resistant proliferation that are hallmarks of the K303R ERα mutation. An optimized delivery method is established for the peptides using the PULSin™ reagent (Giordano et al., 2009), and anticipate that multiple deliveries are useful throughout the course of the growth assays, in some embodiments. Thus, novel reagents are developed at least to examine the role of the hinge region of ERα as an important determinant of resistance, and also as a new therapeutic target for reversing resistance, first in vitro in models, and if successful in vitro, then used for effects on tumor growth in preclinical xenograft models in athymic nude mice.

In certain embodiments, MAbs to the K303R specific site mutation of ERα are generated. Similar approaches may be used to generate MAbs that specifically detect an epitope dependent on substitution of arginine for lysine 303 (K303R). Synthetic peptides corresponding to sequences in the hinge region of ER between aa 297 to 308 with substitution of arginine for lysine 303 are conjugated to KLH and used as immunogen in mice. One can also prepare peptides with single methylation or acetylation groups at K302 in the context of K303R, as an alternative to enhance antigenicity of the arginine substitution at 303. Hybridomas are screened by the same strategy as above for MAbs with differential reactivity against WT ER peptide vs. the peptide with the K303R substitution by ELISA, and in the context of full length ERα expressed in cells by transfection.

### REFERENCES

All patents and publications mentioned in the specification are indicative of the levels of those skilled in the art to which the invention pertains. All patents and publications are herein incorporated by reference to the same extent as if each individual publication was specifically and individually indicated to be incorporated by reference.

### PATENTS

U.S. Pat. No. 3,817,837, issued Jun. 18, 1974
U.S. Pat. No. 3,850,752, issued Nov. 26, 1974
U.S. Pat. No. 3,939,350, issued Feb. 17, 1976
U.S. Pat. No. 3,996,345, issued Dec. 7, 1976
U.S. Pat. No. 4,196,265, issued Apr. 1, 1980
U.S. Pat. No. 4,215,051, issued Jul. 29, 1980
U.S. Pat. No. 4,275,149, issued Jun. 23, 1981
U.S. Pat. No. 4,277,437, issued Jul. 7, 1981
U.S. Pat. No. 4,366,241, issued Dec. 28, 1982
U.S. Pat. No. 4,340,535, issued Jul. 20, 1982
U.S. Pat. No. 4,472,509, issued Sep. 18, 1984
U.S. Pat. No. 4,554,101, issued Nov. 19, 1985
U.S. Pat. No. 4,683,195, issued Jul. 28, 1987
U.S. Pat. No. 4,683,202, issued Jul. 28, 1987
U.S. Pat. No. 4,740,463, issued Apr. 26, 1988
U.S. Pat. No. 4,800,159, issued Jan. 24, 1989
U.S. Pat. No. 4,816,567, issued Mar. 28, 1989
U.S. Pat. No. 4,867,973, issued Septemer 19, 1989
U.S. Pat. No. 4,883,750, issued Nov. 28, 1989
U.S. Pat. No. 4,938,948, issued Jul. 3, 1990
U.S. Pat. No. 5,021,236, issued Jun. 4, 1991
U.S. Pat. No. 5,138,045, issued Aug. 11, 1992
U.S. Pat. No. 5,196,066, issued Mar. 23, 1993
U.S. Pat. No. 5,262,311, issued Nov. 16, 1993
U.S. Pat. No. 5,279,721, issued Jan. 18, 1994
U.S. Pat. No. 5,342,774, issued Aug. 30, 1994
U.S. Pat. No. 5,565,332, issued Oct. 15, 1996
U.S. Pat. No. 5,633,365, issued May 27, 1997
U.S. Pat. No. 5,665,549, issued Sep. 9, 1997
U.S. Pat. No. 5,693,762, issued Dec. 2, 1997
U.S. Pat. No. 5,840,873, issued Nov. 24, 1998
U.S. Pat. No. 5,843,640, issued Dec. 1, 1998
U.S. Pat. No. 5,843,650, issued Dec. 1, 1998
U.S. Pat. No. 5,843,651, issued Dec. 1, 1998
U.S. Pat. No. 5,843,663, issued Dec. 1, 1998
U.S. Pat. No. 5,846,708, issued Dec. 8, 1998
U.S. Pat. No. 5,846,709, issued Dec. 8, 1998
U.S. Pat. No. 5,846,717, issued Dec. 8, 1998
U.S. Pat. No. 5,846,726, issued Dec. 8, 1998
U.S. Pat. No. 5,846,729, issued Dec. 8, 1998
U.S. Pat. No. 5,846,783, issued Dec. 8, 1998
U.S. Pat. No. 5,849,481, issued Dec. 15, 1998
U.S. Pat. No. 5,849,483, issued Dec. 15, 1998
U.S. Pat. No. 5,849,486, issued Dec. 15, 1998
U.S. Pat. No. 5,849,487, issued Dec. 15, 1998
U.S. Pat. No. 5,849,497, issued Dec. 15, 1998
U.S. Pat. No. 5,849,546, issued Dec. 15, 1998
U.S. Pat. No. 5,849,547, issued Dec. 15, 1998
U.S. Pat. No. 5,851,770, issued Dec. 22, 1998
U.S. Pat. No. 5,851,772, issued Dec. 22, 1988
U.S. Pat. No. 5,853,990, issued Dec. 29, 1998
U.S. Pat. No. 5,853,993, issued Dec. 29, 1998
U.S. Pat. No. 5,853,992, issued Dec. 29, 1998
U.S. Pat. No. 5,856,092, issued Jan. 5, 1999
U.S. Pat. No. 5,858,652, issued Jan. 12, 1999
U.S. Pat. No. 5,861,244, issued Jan. 19, 1999
U.S. Pat. No. 5,863,732, issued Jan. 26, 1999
U.S. Pat. No. 5,863,753, issued Jan. 26, 1999
U.S. Pat. No. 5,866,331, issued Feb. 2, 1999
U.S. Pat. No. 5,866,336, issued Feb. 2, 1999
U.S. Pat. No. 5,866,337, issued Feb. 2, 1999
U.S. Pat. No. 5,900,481, issued May 4, 1999
U.S. Pat. No. 5,905,024, issued May 18, 1999
U.S. Pat. No. 5,910,407, issued Jun. 8, 1999
U.S. Pat. No. 5,912,124, issued Jun. 15, 1999
U.S. Pat. No. 5,912,145, issued Jun. 15, 1999
U.S. Pat. No. 5,912,148, issued Jun. 15, 1999
U.S. Pat. No. 5,916,776, issued Jun. 29, 1999
U.S. Pat. No. 5,916,779, issued Jun. 29, 1999
U.S. Pat. No. 5,919,626, issued Jul. 6, 1999
U.S. Pat. No. 5,919,630, issued Jul. 6, 1999
U.S. Pat. No. 5,922,574, issued Jul. 13, 1999
U.S. Pat. No. 5,925,517, issued Jul. 20, 1999
U.S. Pat. No. 5,925,525, issued Jul. 20, 1999
U.S. Pat. No. 5,928,862, issued Jul. 27, 1999
U.S. Pat. No. 5,928,869, issued Jul. 27, 1999
U.S. Pat. No. 5,928,870, issued, Jul. 27, 1999
U.S. Pat. No. 5,928,905, issued Jul. 27, 1999
U.S. Pat. No. 5,928,906, issued Jul. 27, 1999
U.S. Pat. No. 5,929,227, issued Jul. 27, 1999
U.S. Pat. No. 5,932,413, issued Aug. 3, 1999
U.S. Pat. No. 5,932,451, issued Aug. 3, 1999
U.S. Pat. No. 5,935,791, issued Aug. 10, 1999
U.S. Pat. No. 5,935,825, issued Aug. 10, 1999
U.S. Pat. No. 5,939,291, issued Aug. 17, 1999
U.S. Pat. No. 5,942,391, issued Aug. 24, 1999
U.S. Pat. No. 6,803,206, issued Oct. 12, 2004
European Application No. 320 308
European Application No. 329 822
European Application No. EP 431,523
European Application No. EP 329,822
GB Application No. 2 202 328
PCT Application No. PCT/US87/00880
PCT Application No. PCT/US89/01025
PCT Application WO 88/10315
PCT Application WO 89/06700
PCT Application WO 90/07641
PCT Application No. PCT/US87/00880
PCT Application No. PCT/US89/01025
PCT Application WO 88/10315
PCT Application WO 89/06700
PCT Application WO 94/10343
PCT Application WO 94/23050
PCT Application WO 95/10265

### Publications

Agoulnik I U, Tong X W, Fischer D C, et al: A germline variation in the progesterone receptor gene increases transcriptional activity and may modify ovarian cancer risk. J Clin Endocrinol Metab 89:6340-7, 2004
Arpino G, Green S J, Allred D C et al. HER-2 amplification, HER-1 expression, and tamoxifen response in estrogen receptor-positive metastatic breast cancer: a Southwest Oncology Group Study. Clin Cancer Res 2004; 10:5670-6.
Arpino G, Wiechmann L, Osborne C K, et al. (2008) Crosstalk between the estrogen receptor and the HER tyrosine kinase receptor family: molecular mechanism and clinical implications for endocrine therapy resistance. Endocr Rev 29:217-33
Badve S S, Baehner F L, Gray R P, et al: Estrogen- and progesterone-receptor status in ECOG 2197: comparison of immunohistochemistry by local and central laboratories and quantitative reverse transcription polymerase chain reaction by central laboratory. J Clin Oncol 26:2473-81, 2008
Balasenthil S, Barnes C J, Rayala S K, et al. (2004) Estrogen receptor activation at serine 305 is sufficient to upregulate cyclin D1 in breast cancer cells. FEBS Lett 567:243-7
Balleine R L, Earl M J, Greenberg M L, et al. (1999) Absence of progesterone receptor associated with secondary breast cancer in postmenopausal women. Br J Cancer 79:1564-71
Bardou V J, Arpino G, Elledge R M, et al. (2003) Progesterone receptor status significantly improves outcome prediction over estrogen receptor status alone for adjuvant endocrine therapy in two large breast cancer databases. J Clin Oncol 21:1973-9
Benz C C, Scott G K, Sarup J C, et al. (1992) Estrogen-dependent, tamoxifen-resistant tumorigenic growth of MCF-7 cells transfected with HER2/neu. Breast Cancer Res Treat 24:85-95
Berno V, Hinojos C A, Amazit L, et al. (2006) High-resolution, high-throughput microscopy analyses of nuclear receptor and coregulator function. Methods Enzymol 414:188-210
Berry D A, Muss H B, Thor A D, et al. (2000) HER-2/neu and p53 expression versus tamoxifen resistance in estrogen receptor-positive, node-positive breast cancer. J Clin Oncol 18:3471-9
Bjornstrom L, Sjoberg M (2005) Mechanisms of estrogen receptor signaling: convergence of genomic and nongenomic actions on target genes. Mol Endocrinol 19:833-42
Britton D J, Hutcheson I R, Knowlden J M, et al. (2006) Bidirectional cross talk between ERalpha and EGFR signalling pathways regulates tamoxifen-resistant growth. Breast Cancer Res Treat 96:131-46
Brunner N, Frandsen T L, Holst-Hansen C, et al. (1993) MCF7/LCC2: a 4-hydroxytamoxifen resistant human breast cancer variant that retains sensitivity to the steroidal antiestrogen ICI 182,780. Cancer Res 53:3229-32
Carter P, Presta L, Gorman C M, et al. (1992) Humanization of an anti-p185HER2 antibody for human cancer therapy. Proc Natl Acad Sci USA 89:4285-9
Castano E, Vorojeikina D P, Notides A C. Phosphorylation of serine-167 on the human oestrogen receptor is important for oestrogen response element binding and transcriptional activation. Biochem J 1997; 326:149-57.
Chen S, S Masri, X Wang, S Phung, Y C Yuan and X Wu. What do we know about the mechanisms of aromatase inhibitor resistance? J. Steroid Biochem. Mol. Biol. 102 (2006), pp. 232-40.
Chung Y L, Sheu M L, Yang S C, et al. (2002) Resistance to tamoxifen-induced apoptosis is associated with direct interaction between Her2/neu and cell membrane estrogen receptor in breast cancer. Int J Cancer 97:306-12
Collins L C, Botero M L, Schnitt S J: Bimodal frequency distribution of estrogen receptor immunohistochemical staining results in breast cancer: an analysis of 825 cases. Am J Clin Pathol 123:16-20, 2005
Conway K, Parrish E, Edmiston S N, et al. (2007) Risk factors for breast cancer characterized by the estrogen receptor alpha A908G (K303R) mutation. Breast Cancer Res 9:R36
Conway K, Parrish E, Edmiston S N, et al: The estrogen receptor-alpha A908G (K303R) mutation occurs at a low frequency in invasive breast tumors: results from a population-based study. Breast Cancer Res 7:R871-80, 2005
Cui X, Schiff R, Arpino G, et al. (2005) Biology of progesterone receptor loss in breast cancer and its implications for endocrine therapy. J Clin Oncol 23:7721-35
Cui X, Zhang P, Deng W, et al. (2003) Insulin-like growth factor-I inhibits progesterone receptor expression in breast cancer cells via the phosphatidylinositol 3-kinase/Akt/mammalian target of rapamycin pathway: progesterone receptor as a potential indicator of growth factor activity in breast cancer. Mol Endocrinol 17:575-88
Cui Y, Zhang M, Pestell R, Curran E M, Welshons W V, Fuqua S A. Phosphorylation of estrogen receptor αblocks its acetylation and regulates estrogen sensitivity. Cancer Research 2004; 64:9199-9208.
Dauvois S, White R, Parker M G. The antiestrogen ICI 182780 disrupts estrogen receptor nucleocytoplasmic shuttling. J Cell Sci 1993; 106:1377-88.
Davies M P, O′Neill P A, Innes H, et al: Hypersensitive K303R oestrogen receptor-alpha variant not found in invasive carcinomas. Breast Cancer Res 7:R113-8, 2005
De Laurentiis M, Arpino G, Massarelli E, et al. (2005) A meta-analysis on the interaction between HER-2 expression and response to endocrine treatment in advanced breast cancer. Clin Cancer Res 11:4741-8
Dowsett M, Harper-Wynne C, Boeddinghaus I, et al. (2001) HER-2 amplification impedes the antiproliferative effects of hormone therapy in estrogen receptor-positive primary breast cancer. Cancer Res 61:8452-8
Elledge R M, Green S, Pugh R, et al. (2000) Estrogen receptor (ER) and progesterone receptor (PgR), by ligand-binding assay compared with ER, PgR and pS2, by immuno-histochemistry in predicting response to tamoxifen in metastatic breast cancer: a Southwest Oncology Group Study. Int J Cancer 89:111-7
Encarnacion C A, Ciocca D R, McGuire W L, et al. (1993) Measurement of steroid hormone receptors in breast cancer patients on tamoxifen. Breast Cancer Res Treat 26:237-46
Fan P, Wang J, Santen R J, et al. (2007) Long-term treatment with tamoxifen facilitates translocation of estrogen receptor alpha out of the nucleus and enhances its interaction with EGFR in MCF-7 breast cancer cells. Cancer Res 67:1352-60
Fuqua S A, Schiff R, Parra I, et al: Expression of wild-type estrogen receptor b and variant isoforms in human breast cancer. Cancer Res 59:5425-5428, 1999
Fuqua S A, C Wiltschke, Q X Zhang, A Borg, C G Castles, W E Friedrichs, T Hopp, S Hilsenbeck, S Mohsin, P O'Connell P and D C Allred. A hypersensitive estrogen receptor-alpha mutation in premalignant breast lesions. Cancer Res. 60 (2000), pp. 4026-4029.
Fuqua S A W, Falette N F, McGuire W L: Sensitive detection of estrogen receptor RNA by polymerase chain reaction assay. Journal of the National Cancer Institute 82:858-861, 1990
Gee, J M, Robertson J F, Ellis I O. Phosphorylation of ERK1/2 mitogenic-activated protein kinase is associated with poor response to anti-hormonal therapy and decreased patient survival in clinical breast cancer. Int J Cancer 2001; 95:247-254.
Giordano C, Cui Y, Barone I, et al: Growth factor-induced resistance to tamoxifen is associated with a mutation of estrogen receptor alpha and its phosphorylation at serine 305. Breast Cancer Res Treat, Epub 2009 Feb. 11.
Goss P E, Ingle J N, Martino S, et al. (2007) Efficacy of letrozole extended adjuvant therapy according to estrogen receptor and progesterone receptor status of the primary tumor: National Cancer Institute of Canada Clinical Trials Group MA.17. J Clin Oncol 25:2006-11
Harris L, Fritsche H, Mennel R, et al: American Society of Clinical Oncology 2007 update of recommendations for the use of tumor markers in breast cancer. J Clin Oncol 25:5287-312, 2007
Herynk M H, Beyer A R, Cui Y, et al. (2006) Cooperative action of tamoxifen and c-Src inhibition in preventing the growth of estrogen receptor-positive human breast cancer cells. Mol Cancer Ther 5:3023-31
Herynk M H, Fuqua S A (2004) Estrogen receptor mutations in human disease. Endocr Rev 25:869-98
Herynk M H, Parra I, Cui Y, et al. (2007) Association between the estrogen receptor alpha A908G mutation and outcomes in invasive breast cancer. Clin Cancer Res 13:3235-43
Hilsenbeck S G, Ravdin P M, de Moor C A, et al: Time-dependence of hazard ratios for prognostic factors in primary breast cancer. Breast Cancer Res. Treatment 52:227-237, 1998
Holm C, Kok M, Michalides R, et al: Phosphorylation of the oestrogen receptor alpha at serine 305 and prediction of tamoxifen resistance in breast cancer. J Pathol 217:372-9, 2009
Hopp T A, Weiss H L, Hilsenbeck S G, et al. (2004) Breast cancer patients with progesterone receptor PR-A-rich tumors have poorer disease-free survival rates. Clin Cancer Res 10:2751-60
Hutcheson I R, Knowlden J M, Madden T A, et al. (2003) Oestrogen receptor-mediated modulation of the EGFR/MAPK pathway in tamoxifen-resistant MCF-7 cells. Breast Cancer Res Treat 81:81-93
Jaremko M, Justenhoven C, Abraham B K, et al: MALDI-TOF MS and TaqMan assisted SNP genotyping of DNA isolated from formalin-fixed and paraffin-embedded tissues (FFPET). Hum Mutat 25:232-8, 2005
Jelovac D, Sabnis G, Long B J, Macedo L, Goloubeva O G, Brodie A M. Activation of mitogen-activated protein kinase in xenografts and cells during prolonged treatment with aromatase inhibitor letrozole. Cancer Res 2005; 65:5380-5389.
Joel P B, Smith J, Sturgill T W, Fisher T L, Blenis J, Lannigan D A. pp90rsk1 regulates estrogen receptormediated transcription through phosphorylation of Ser-167. Mol Cell Biol 1998; 18:1978-84.
Karas R H, Gauer E A, Bieber H E, et al. (1998) Growth factor activation of the estrogen receptor in vascular cells occurs via a mitogen-activated protein kinase-independent pathway. J Clin Invest 101:2851-61
Kato S, Endoh H, Masuhiro Y, et al. (1995) Activation of the estrogen receptor through phosphorylation by mitogen-activated protein kinase. Science 270:1491-4
Kim D, Dan H C, Park S, et al. AKT/PKB signaling mechanisms in cancer and chemoresistance. Front Biosci 2005; 10:975-87.
Kim M Y, Woo E M, Chong Y T, et al. (2006) Acetylation of estrogen receptor alpha by p300 at lysines 266 and 268 enhances the deoxyribonucleic acid binding and transactivation activities of the receptor. Mol Endocrinol 20:1479-93
Kurokawa H, Lenferink A E, Simpson J F, et al. (2000) Inhibition of HER2/neu (erbB-2) and mitogen-activated protein kinases enhances tamoxifen action against HER2-overexpressing, tamoxifen-resistant breast cancer cells. Cancer Res 60:5887-94
Lange C A, Shen T, Horwitz K B (2000) Phosphorylation of human progesterone receptors at serine-294 by mitogen-activated protein kinase signals their degradation by the 26S proteasome. Proc Natl Acad Sci USA 97:1032-7
Lannigan D A (2003), Estrogen receptor phosphorylation. Steroids 68:1-9
Lee A V, Cui X, Oesterreich S (2001) Cross-talk among estrogen receptor, epidermal growth factor, and insulin-like growth factor signaling in breast cancer. Clin Cancer Res 7:4429s-4435s; discussion 11s-12s
Lephart E D, Simpson E R. Assay of aromatase activity. Methods Enzymol 1991; 206:477-83.
Levin E R (2003), Bidirectional signaling between the estrogen receptor and the epidermal growth factor receptor. Mol Endocrinol 17:309-17
Likhite V S, Stossi F, Kim K, et al. (2006) Kinase-specific phosphorylation of the estrogen receptor changes receptor interactions with ligand, deoxyribonucleic acid, and coregulators associated with alterations in estrogen and tamoxifen activity. Mol Endocrinol 20:3120-32
Ma X J, Hilsenbeck S G, Wang W, et al: The HOXB13:IL17BR expression index is a prognostic factor in early-stage breast cancer. J Clin Oncol 24:4611-9, 2006
Mangelsdorf D J, Thummel C, Beato M, et al. (1995) The nuclear receptor superfamily: the second decade. Cell 83:835-9
Martin L A, I Farmer, S R Johnston, S Ali and M Dowsett. Elevated ERK1/ERK2/estrogen receptor cross-talk enhances estrogen-mediated signaling during long-term estrogen deprivation, Endocr. Relat. Cancer 12 (2005), Suppl. 1.
Martin L A, Farmer I, Johnston S R, Ali S, Marshall C, Dowsett M. Enhanced estrogen receptor (ER) a, ERBB2, and MAPK signal transduction pathways operate during the adaptation of MCF-7 cells to long term estrogen deprivation. J Biol Chem 2003; 278:30458-68.
Martin M B, Franke T F, Stoica G E, et al. (2000) A role for Akt in mediating the estrogenic functions of epidermal growth factor and insulin-like growth factor I. Endocrinology 141:4503-11
Michalides R, Griekspoor A, Balkenende A, et al. Tamoxifen resistance by a conformational arrest of the estrogen receptor alpha after PKA activation in breast cancer. Cancer Cell 2004; 6:597-605.
Migliaccio A, Di Domenico M, Castoria G, et al. (2005) Steroid receptor regulation of epidermal growth factor signaling through Src in breast and prostate cancer cells: steroid antagonist action. Cancer Res 65:10585-93
Nakajima H, Mizuta N, S akaguchi K, et al: Development of HER2-antagonistic peptides as novel anti-breast cancer drugs by in silico methods. Breast Cancer 15:65-72, 2008
Nawaz Z, Lonard D M, Dennis A P, et al. (1999) Proteasome-dependent degradation of the human estrogen receptor. Proc Natl Acad Sci USA 96:1858-62
Nicholson R I, Staka C, Boyns F, Hutcheson I R, Gee J M. Growth factor-driven mechanisms associated with resistance to estrogen deprivation in breast cancer: new opportunities for therapy. Endocr Relat Cancer 2004; 11:623-641.
Nicholson R I, Gee J M, Knowlden J, et al. (2003) The biology of antihormone failure in breast cancer. Breast Cancer Res Treat 80 Suppl 1:S29-34; discussion S5
Nicholson R I, Hutcheson I R, Harper M E, et al. (2001) Modulation of epidermal growth factor receptor in endocrine-resistant, oestrogen receptor-positive breast cancer. Endocr Relat Cancer 8:175-82
Nicholson R I, McClelland R A, Robertson J F et al. Involvement of steroid hormone and growth factor cross-talk in endocrine response in breast cancer. Endocr Relat Cancer 1999; 6:373-387.
Nicholson S, Halcrow P, Sainsbury J R, et al. (1988) Epidermal growth factor receptor (EGFr) status associated with failure of primary endocrine therapy in elderly postmenopausal patients with breast cancer. Br J Cancer 58:810-4
O'Malley B W (2006), Molecular biology. Little molecules with big goals. Science 313:1749-50
Osborne C K, Coronado-Heinsohn E B, Hilsenbeck S G, McCue B L, Wakeling A E, McClelland R A. Comparison of the effects of a pure steroidal antiestrogen with those of tamoxifen in a model of human breast cancer. J Natl Cancer Inst 1995; 87:746-50.
Osborne C K, Bardou V, Hopp T A, et al. (2003) Role of the estrogen receptor coactivator AIB1 (SRC-3) and HER-2/neu in tamoxifen resistance in breast cancer. J Natl Cancer Inst 95:353-61
Paik S, Shak S, Tang G, et al: A multigene assay to predict recurrence of tamoxifen-treated, node-negative breast cancer. N Engl J Med 351:2817-26, 2004
Perez-Tenorio G, Stal O. Activation of AKT/PKB in breast cancer predicts a worse outcome among endocrine treated patients. Br J Cancer 2002; 86:540-545.
Pijnenborg J M, Romano A, Dam-de Veen G C, et al: Aberrations in the progesterone receptor gene and the risk of recurrent endometrial carcinoma. J Pathol 205:597-605, 2005
Pink J J, Jordan V C. Models of estrogen receptor regulation by estrogens and antiestrogens in breast cancer cell lines. Cancer Res 1996; 56:2321-30.
Ravdin P M, Green S, Dorr T M, et al. (1992) Prognostic significance of progesterone receptor levels in estrogen receptor-positive patients with metastatic breast cancer treated with tamoxifen: results of a prospective Southwest Oncology Group study. J Clin Oncol 10:1284-91
Rayala S K, Talukder A H, Balasenthil S, et al. (2006) P21-activated kinase 1 regulation of estrogen receptor-alpha activation involves serine 305 activation linked with serine 118 phosphorylation. Cancer Res 66:1694-701
Razandi M, Alton G, Pedram A, et al. (2003) Identification of a structural determinant necessary for the localization and function of estrogen receptor alpha at the plasma membrane. Mol Cell Biol 23:1633-46
Romond E H, Perez E A, Bryant J, et al. (2005) Trastuzumab plus adjuvant chemotherapy for operable HER2-positive breast cancer. N Engl J Med 353:1673-84
Sabnis G J, Jelovac D, Long B, Brodie A. The role of growth factor receptor pathways in human breast cancer cells adapted to long-term estrogen deprivation. Cancer Res 2005; 65:3903-3910.
Samanta A, LeVea C M, Dougall W C, et al. (1994) Ligand and p185c-neu density govern receptor interactions and tyrosine kinase activation. Proc Natl Acad Sci USA 91:1711-5
Santen R J, R X Song, Z Zhang, R Kumar, M H Jeng, S Masamura, J Lawrence Jr., L P MacMahon, W Yue and L Berstein. Adaptive hypersensitivity to estrogen: mechanisms and clinical relevance to aromatase inhibitor therapy in breast cancer treatment, J. Steroid Biochem. Mol. Biol. 95 (2005), pp. 155-165.
Schiff R, Massarweh S, Shou J, et al. (2003) Breast cancer endocrine resistance: how growth factor signaling and estrogen receptor coregulators modulate response. Clin Cancer Res 9:447 S-54S
Schiff R, Massarweh S A, Shou J, et al. (2004) Cross-talk between estrogen receptor and growth factor pathways as a molecular target for overcoming endocrine resistance. Clin Cancer Res 10:331 S-6S
Schmoor C, Sauerbrei W, Schumacher M: Sample size considerations for the evaluation of prognostic factors in survival analysis. Stat Med 19:441-52, 2000
Sentis S, Le Romancer M, Bianchin C, et al. (2005) Sumoylation of the estrogen receptor alpha hinge region regulates its transcriptional activity. Mol Endocrinol 19:2671-84
Sharp Z D, Mancini M G, Hinojos C A, et al. (2006) Estrogen-receptor-alpha exchange and chromatin dynamics are ligand- and domain-dependent. J Cell Sci 119:4101-16
Shim W S, Conaway M, Masamura S, et al. Estradiol hypersensitivity and mitogen-activated protein kinase expression in long-term estrogen deprived human breast cancer cells in vivo . Endocrinology. 1999/12/30 ed; 2000. p. 396-405.
Shou J, Massarweh S, Osborne C K, et al. (2004) Mechanisms of tamoxifen resistance: increased estrogen receptor-HER2/neu cross-talk in ER/HER2-positive breast cancer. J Natl Cancer Inst 96:926-35
Simoncini T, Hafezi-Moghadam A, Brazil D P, Ley K, Chin W W, Liao J K. Interaction of oestrogen receptor with the regulatory subunit of phosphatidylinositol-3-OH kinase. Nature 2000; 407:538-41.
Slamon D J, Godolphin W, Jones L A, et al. (1989) Studies of the HER-2/neu proto-oncogene in human breast and ovarian cancer. Science 244:707-12
Smith C L, Conneely O M, O'Malley B W (1993) Modulation of the ligand-independent activation of the human estrogen receptor by hormone and antihormone. Proc Natl Acad Sci USA 90:6120-4
Song G, Ouyang G, Bao S. The activation of Akt/PKB signaling pathway and cell survival. J Cell Mol Med 2005; 9:59-71.
Song R X, McPherson R A, Adam L, et al. (2002) Linkage of rapid estrogen action to MAPK activation by ERalpha-Shc association and Shc pathway activation. Mol Endocrinol 16:116-27
Song R X, Santen R J (2006) Membrane initiated estrogen signaling in breast cancer. Biol Reprod 75:9-16
Staka C M, Nicholson R I, Gee J M. Acquired resistance to oestrogen deprivation: role for growth factor signalling kinases/oestrogen receptor cross-talk revealed in new MCF-7X model. Endocr Relat Cancer 2005; 12 Suppl 1:S85-97.
Su J L, McKee D D, Ellis B, et al: Production and characterization of an estrogen receptor b subtype-specific mouse monoclonal antibody. Hybridoma 19:481-487, 2000
Subramanian K, Jia D, Kapoor-Vazirani P, et al. (2008) Regulation of estrogen receptor alpha by the SET7 lysine methyltransferase. Mol Cell 30:336-47
Sun M, Paciga J E, Feldman R1, et al. (2001) Phosphatidylinositol-3-OH Kinase (PI3K)/AKT2, activated in breast cancer, regulates and is induced by estrogen receptor alpha (ERalpha) via interaction between ERalpha and PI3K. Cancer Res 61:5985-91
Thiantanawat A, Long B J, Brodie A M. Signaling pathways of apoptosis activated by aromatase inhibitors and antiestrogens. Cancer Res 2003; 63:8037-50.
Tokunaga E, Oki E, Nishida K, et al. (2006) Trastuzumab and breast cancer: developments and current status. Int J Clin Oncol 11:199-208
Varricchio L, Migliaccio A, Castoria G, et al. (2007) Inhibition of estradiol receptor/Src association and cell growth by an estradiol receptor alpha tyrosine-phosphorylated peptide. Mol Cancer Res 5:1213-21
Vogel C L, Cobleigh M A, Tripathy D, et al. (2002) Efficacy and safety of trastuzumab as a single agent in first-line treatment of HER2-overexpressing metastatic breast cancer. J Clin Oncol 20:719-26
Wang C, Fu M, Angeletti R H, et al: Direct acetylation of the estrogen receptor alpha hinge region by p300 regulates transactivation and hormone sensitivity. J Biol Chem 276:18375-83, 2001
Wang L M, Kuo A, Alimandi M, et al. (1998) ErbB2 expression increases the spectrum and potency of ligand-mediated signal transduction through ErbB4. Proc Natl Acad Sci USA 95:6809-14
Wang R A, Mazumdar A, Vadlamudi R K, et al. (2002) P21-activated kinase-1 phosphorylates and transactivates estrogen receptor-alpha and promotes hyperplasia in mammary epithelium. EMBO J 21:5437-47
Wong C W, McNally C, Nickbarg E, et al. (2002) Estrogen receptor-interacting protein that modulates its nongenomic activity-crosstalk with Src/Erk phosphorylation cascade. Proc Natl Acad Sci USA 99:14783-8
Worthylake R, Opresko L K, Wiley H S (1999) ErbB-2 amplification inhibits down-regulation and induces constitutive activation of both ErbB-2 and epidermal growth factor receptors. J Biol Chem 274:8865-74
Yeon C H, Pegram M D (2005) Anti-erbB-2 antibody trastuzumab in the treatment of HER2-amplified breast cancer. Invest New Drugs 23:391-409
Zwart W, Griekspoor A, Berno V, et al. (2007) PKA-induced resistance to tamoxifen is associated with an altered orientation of ERalpha towards co-activator SRC-1. EMBO J. 26:3534-44
Zwart W, Griekspoor A, Berno V, et al. PKA-induced resistance to tamoxifen is associated with an altered orientation of ERalpha towards co-activator SRC-1. EMBO J. 2007, 15:3534-44. Epub 2007 Jul. 12.

One skilled in the art readily appreciates that the patent invention is well adapted to carry out the objectives and obtain the ends and advantages mentioned as well as those inherent therein. Mutations, kits, sequences, methods, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

What is claimed is:

1. A method of identifying a cancer in an individual that is susceptible to becoming resistant to hormonal breast cancer therapy, will become resistant to hormonal breast cancer therapy, or that is resistant to hormonal breast cancer therapy, comprising the step of assaying a sample from the individual for an A908G mutation in estrogen receptor alpha (ERα) sequence, wherein the presence of said mutation in said nucleic acid sequence indicates said individual is susceptible to becoming resistant to hormonal breast cancer therapy, will become resistant to hormonal breast cancer therapy, or is resistant to hormonal breast cancer therapy.

2. The method of claim 1, wherein the hormonal breast cancer therapy comprises one or more antiestrogens.

3. The method of claim 1, wherein the hormonal breast cancer therapy comprises one or more aromatase inhibitors.

4. The method of claim 1, wherein the method further comprises providing an alternative breast cancer therapy to the individual.

5. The method of claim 1, wherein said sample is obtained by biopsy.

6. The method of claim 1, wherein said assaying step comprises primer extension, sequencing, single stranded conformation polymorphism, mismatch oligonucleotide mutation detection, mass spectroscopy, DNA microarray, HPLC, microarray, SNP PCR genotyping, or a combination thereof.

7. The method of claim 1, wherein said assaying step is by antibody detection with antibodies to said A908G mutation of said estrogen receptor alpha nucleic acid sequence or is by antibody detection with antibodies to a corresponding estrogen receptor alpha K303R amino acid sequence.

8. The method of claim 7, wherein the antibodies to the corresponding estrogen receptor alpha K303R amino acid sequence immunologically recognizes a modified K303R amino acid sequence.

9. The method of claim 8, wherein the modified K303R amino acid sequence is acetylated.

10. A peptide comprising the estrogen receptor alpha K303 amino acid.

11. The peptide of claim 10, wherein said peptide is no more than 20 amino acids long, no less than 5-6 amino acids long, or from 5-20 amino acids in length.

12. The peptide of claim 10, wherein there is a mutation at the estrogen receptor alpha S305 amino acid.

13. The peptide of claim 10, wherein the peptide is capable of being phosphorylated at the estrogen receptor alpha S305 amino acid.

14. The peptide of claim 10, wherein the peptide is not capable of being phosphorylated at the estrogen receptor alpha S305 amino acid.

15. The peptide of claim 10, wherein the estrogen receptor alpha S305 amino acid is acetylated, sumoylated, ubiquitinated, or methylated.

16. A kit for diagnosing susceptibility or presence of resistance to a hormonal breast cancer therapy in an individual, comprising one or more reagents for detecting an A908G mutation in an estrogen receptor alpha nucleic acid sequence and an alternative breast cancer therapy.

17. A method of identifying a cancer in an individual that is sensitive to breast cancer chemotherapy, comprising the step of assaying a sample from the individual for an A908G mutation in estrogen receptor alpha (ERα) sequence, wherein the presence of said mutation in said nucleic acid sequence indicates said individual is sensitive breast cancer chemotherapy.

18. A method of determining a treatment regimen for an individual suspected of having breast cancer, comprising the steps of:
assaying a sample from the individual for an A908G mutation in estrogen receptor alpha (ERα) sequence, wherein the presence of said mutation in said nucleic acid sequence indicates said individual is susceptible to becoming resistant to hormonal breast cancer therapy, will become resistant to hormonal breast cancer therapy, or is resistant to hormonal breast cancer therapy; and
determining a treatment regimen for the individual based on the outcome of said assaying step.

19. The method of claim 18, wherein the treatment regimen comprises an alternative to hormonal breast cancer therapy.

20. The method of claim 19, wherein the alternative to hormonal breast cancer therapy comprises therapy selected from the group consisting of targeted therapy to other molecular alterations in the breast cancer, targeted therapy with signal transduction inhibitors, fulvestrant, chemotherapy, or a steroidal aromatase inhibitor.

21. The method of claim 20, wherein the targeted therapy to other molecular alterations in the breast cancer targets HER2, EGFR, IGF1R, PI3K, or AKT.

22. The method of claim 20, wherein the targeted therapy with signal transduction inhibitors targets PI3K or AKT.

23. The method of claim 20, wherein the steroidal aromatase inhibitor comprises exemestane.

24. A method of determining response to an aromatase inhibitor in a breast tumor comprising the step of assaying an A908G mutation in estrogen receptor alpha (ERα) sequence from cells from the tumor, wherein when the cells comprise the mutation the breast tumor is or will become resistant to the aromatase inhibitor.


SEQUENCE LISTING (27 sequences)

SEQ ID NO: 1 - DNA, 6330 bp, Homo sapiens
aggagctggcggagggcgttcgtcctgggactgcacttgctcccgtcgggtcgcccggcttcaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcattccccctataggaatacaaggggcacacagggaaggcagatcccctagttggcaagactattttaacttgatacactgcagattcagatgtgctgaaagctctgcctctggctttccggtcatgggttccagttaattcatgcctcccatggacctatggagagcagcaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgaggcacagccagacttgctcagggtggccctgccacaggctgcagctacctaggaacattccttgcagaccccgcattgccctttgggggtgccctgggatccctggggtagtccagctcttcttcatttcccagcgtggccctggttggaagaagcagctgtcacagctgctgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtgagctgctcgggcttctcttggtatgtcttgtttggaaaagtggatttcattcatttctgattgtccagttaagtgatcaccaaaggactgagaatctgggagggcaaaaaaaaaaaaaaagtttttatgtgcacttaaatttggggacaattttatgtatctgtgttaaggatatgtttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatactgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccagggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaatagagtcatacagtagctcaaaaggcaaccataattctctttggtgcaggtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcttttttttgcatccaattgtgcctgaacttttaaaatatgtaaatgctgccatgttccaaacccatcgtcagtgtgtgtgtttagagctgtgcaccctagaaacaacatattgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaactaaa

SEQ ID NO: 2 - DNA, 1644 bp, Artificial Sequence
  Synthetic construct
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgcccggagggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctga

SEQ ID NO: 3 - DNA, 1237 bp, Homo sapiens
tcctcccctgaatcagtttgtatgggctcaccaaagcctactgttcaattttcaggagttttgtaagccatttgtcagacaagtggcctgaagtttgttatggtggtggtatttacaccatgaaaattggcatgttatggtggtagtatttaccccatgaaaactggtacaaattgaaatctttttcttcttctcttggagagccacttgttgaacacttaccagctcacctgtgcttgaaagtatttcttcaaataaaatgaaagctggttagctttgaaaattttttgtctaaaagtttacacgggaaaaaaattaactaattttttttttccacctgtgttttcagggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggtaagaatgcgaagcgcagcttttaagagtcaatagcttttcaagaacttgttgtgatgtcatgggagaaatagtgggggaaaaagaagcaataacatgttatgtaattggtttcaaggttacaggagatgtgttcattttcagtatcaatacactgtaattttccaggagattaggaaataatatttttaaatcagaatctagaagactgaaattcttaaattgacataatttatttttaacccatctcatttaccaaaaagatttagggtggacactacatggtaaaactatttaatagtgtatgttcacagtagcagaaacttttaacactaaatgaactacaaaagtttgtaatattaatgacctttgttgaaaacatctcaattattaatcaaacgattttatcttaaaaagatttttaagattcggtgtggtggctcgtgcctgtaatcctagcactttttggggctgaggtgggaggattgcttgagcccaagagcttgaggaaagagaggacagcgattctcgtacgaacggttacgattctga

SEQ ID NO: 4 - DNA, 21 bp, Artificial Sequence
  Synthetic primer
cagagagaatgatggggaggg

SEQ ID NO: 5 - DNA, 2106 bp, Homo sapiens
atgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctga

SEQ ID NO: 6 - DNA, 2092 bp, Homo sapiens
gaattccaaaattgtgatgtttcttgtatttttgatgaaggagaaatactgtaatgatcactgtttacactatgtacactttaggccagccctttgtagcgttatacaaactgaaagcacaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggc

SEQ ID NO: 7 - DNA, 6450 bp, Homo sapiens
gagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggagctgcacttgctccgtcgggtcgccggcttcaccggaccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcacttccccctataggaatacaaggggccacacagggaaggcagatcccctagttggccaagacttattttaacttgatacactgcagattcagagtgtcctgaagctctgcctctggctttccggtcatgggttccagttaattcatgcctcccatggacctatggagagcaacaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgagcacagccagacttgctcaggtggccctgcacaggctgcagctacctaggaacattccttgcagaccccgcattgcctttgggggtgccctgggatccctggggtagtccagctcttattcatttcccagcgtggccctggttggaagaagcagctgtcaagttgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtagctgctcgggcttctcttggtatgtcttgtttggaaaagtggatttcattcatttctgattgtccagttaagtgatcaccaaaggactgagaatctgggagggcaaaaaaaaaaaaaaaagtttttatgtgcacttaaatttggggacaattttatgtatctgtgttaaggatatgcttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatattgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccaaggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaataaagtcatacagtagctcaaaaggcaaccataattctctttggtgcaagtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcatttttttgcatccaattgtgcctgaacttttaaaatatgtaaatgctgccatgttccaaacccatcgtcagtgtgtgtgtttagagctgtgcaccctagaaacaacatacttgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaact

SEQ ID NO: 8 - protein, 595 aa, Homo sapiens [sequence omitted]

SEQ ID NO: 9 - protein, 547 aa, Artificial Sequence
  Synthetic construct
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMPGGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 10 - protein, 113 aa, Homo sapiens
RRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGTRENV

SEQ ID NO: 11 - protein, 701 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 12 - protein, 595 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 13 - protein, 595 aa, Homo sapiens; identical to SEQ ID NO: 12

SEQ ID NO: 14 - protein, 13 aa, Artificial Sequence
  blocking sequence
IKRSKKNSLALSC

SEQ ID NO: 15 - DNA, 20 bp, Artificial Sequence
  Synthetic Primer
acatgagagctgccaacctt

SEQ ID NO: 16 - DNA, 20 bp, Artificial Sequence
  Synthetic Primer
ggaatagagtatcgggggct

SEQ ID NO: 17 - DNA, 22 bp, Artificial Sequence
  Synthetic Primer
ttcatgatcaaacgctctaaga

SEQ ID NO: 18 - DNA, 17 bp, Artificial Sequence
  Synthetic Primer
acaaggccaggctgttc

SEQ ID NO: 19 - DNA, 20 bp, Artificial Sequence
  Synthetic Primer
caagcgccagagagatgatg

SEQ ID NO: 20 - DNA, 20 bp, Artificial Sequence
  Synthetic Primer
acaaggcactgaccatctgg

SEQ ID NO: 21 - DNA, 20 bp, Artificial Sequence
  Synthetic Primer
gaccatctggtcggccgtca

SEQ ID NO: 22 - DNA, 31 bp, Artificial Sequence
  Synthetic Primer
acactagtatggttttggaaatgctgaaccc

SEQ ID NO: 23 - DNA, 32 bp, Artificial Sequence
  Synthetic Primer
acgcggccgcctagtgttccagacacctgtct

SEQ ID NO: 24 - protein, 13 aa, Artificial Sequence
  Synthetic Construct
IKRSKKNSLALSC

SEQ ID NO: 25 - protein, 12 aa, Artificial Sequence
  Synthetic construct
MIKRSXXNSLAL

SEQ ID NO: 26 - protein, 12 aa, Artificial Sequence
  Synthetic construct
MIKRSXXNSLAL

SEQ ID NO: 27 - protein, 12 aa, Artificial Sequence
  Synthetic construct
KRSKKNXLALSL